(19) United States
(12) Patent Application Publication
(10) Pub. No.: US 2016/0166668 A1
(43) Pub. Date: Jun. 16, 2016

(54) COMPOSITION AND VACCINE FOR TREATING PROSTATE CANCER

(71) Applicant: CureVac AG, Tubingen (DE)

(72) Inventors: Karl-Josef KALLEN, Konigsdorf (DE); Mariola FOTIN-MLECZEK, Sindelfingen (DE); Ulrike GNAD-VOGT, Muhltal (DE); Thomas LANDER, Konigstein i. Taunus (DE)

(73) Assignee: CureVac AG, Tubingen (DE)

(21) Appl. No.: 15/048,126

(22) Filed: Feb. 19, 2016

Related U.S. Application Data
(63) Continuation of application No. PCT/EP2014/002297, filed on Aug. 21, 2014.

(30) Foreign Application Priority Data
Aug. 21, 2013 (EP) PCT/EP2013/002515
Aug. 21, 2013 (EP) PCT/EP2013/002516

(51) Int. Cl. A61K 39/00; C07K 14/47; C12N 9/16; C12N 9/64; C12N 9/48; C07K 14/705; C12N 9/02

(52) U.S. Cl. CPC A61K 39/0011; C07K 14/705; C07K 14/4727; C12N 9/0091; C12N 9/6424; C12N 9/485; C12N 9/16; A61K 2039/53; A61K 48/00

(57) ABSTRACT

The present invention relates to a composition comprising at least one mRNA encoding a combination of antigens capable of eliciting an (adaptive) immune response in a mammal, wherein the antigens are selected from the group consisting of PSA (Prostate-Specific Antigen), PSMA (Prostate-Specific Membrane Antigen), PSCA (Prostate Stem Cell Antigen), STEAP (Six Transmembrane Epithelial Antigen of the Prostate), MUC1 (Mucin 1) and PAP (Prostatic acid phosphatase). The invention furthermore relates to a vaccine comprising at least one mRNA encoding such a combination of antigens and to the use of said composition (for the preparation of a vaccine) and/or of the vaccine for eliciting an (adaptive) immune response for the treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto. Finally, the invention relates to kits, particularly to kits of parts, containing the composition and/or the vaccine.

The present application is a continuation of International Application No. PCT/EP2014/002297, filed Aug. 21, 2014, which claims priority benefit of European Application No. PCT/EP2013/002516, filed Aug. 21, 2013, and European Application No. PCT/EP2013/002515, filed Aug. 21, 2013, the entire text of each of the above referenced disclosures being specifically incorporated herein by reference.

### BACKGROUND OF THE INVENTION

1. Field of the Invention

The present invention relates to a composition comprising at least one mRNA encoding a combination of antigens capable of eliciting an (adaptive) immune response in a mammal, wherein the antigens are selected from the group consisting of PSA (Prostate-Specific Antigen), PSMA (Prostate-Specific Membrane Antigen), PSCA (Prostate Stem Cell Antigen), STEAP (Six Transmembrane Epithelial Antigen of the Prostate), MUC1 (Mucin 1) and PAP (Prostatic acid phosphatase). The invention furthermore relates to a vaccine comprising at least one mRNA encoding such a combination of antigens and to the use of said composition (for the preparation of a vaccine) and/or of the vaccine for eliciting an (adaptive) immune response for the treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto. Finally, the invention relates to kits, particularly to kits of parts, containing the composition and/or the vaccine.

2. Background of the Invention

At present, prostate cancer is the second most commonly diagnosed cancer and the fourth leading cause of cancer-related death in men in the developed countries worldwide. Effective curative treatment modalities are debilitating, and are only currently available for localised disease. In hormone-refractory (castration-resistant; castration-refractory) prostate cancer, no agent has been shown to prolong survival beyond approximately 1 year (see e.g. Pavlenko, M., A. K. Roos, et al. (2004). “A phase I trial of DNA vaccination with a plasmid expressing prostate-specific antigen in patients with hormone-refractory prostate cancer.” Br J Cancer 91(4): 688-94.). In some highly developed western countries such as the United States of America, prostate cancer is at present even the most commonly diagnosed malignancy and the third leading cause of cancer related death among men in the United States (see e.g. Jemal, A., R. Siegel, et al. (2006). “Cancer statistics, 2006.” CA Cancer J Clin 56(2): 106-30.) and in Europe, respectively (see e.g. Thomas-Kaskel, A. K., C. F. Waller, et al. (2007). “Immunotherapy with dendritic cells for prostate cancer.” Int J Cancer 121(3): 467-73). Most of the diagnosed tumors are adeno-carcinomas which initially proliferate in a hormone-dependent manner.

Prostate cancer is a disease in which cancer develops in the prostate, a gland in the male reproductive system. It occurs when cells of the prostate mutate and begin to multiply out of control. Typical antigens which have been shown to be overexpressed by prostate cancer cells as compared to normal counterparts are inter alia antigens like PSA, PSMA, PAP, PSCA, HER-2 and Ep-CAM. These prostate cancer cells may spread (metastasize) from the prostate to other parts of the body, especially the bones and lymph nodes. Prostate cancer may cause pain, difficulty in urinating, erectile dysfunction and other symptoms. Typically, prostate cancer develops most frequently in men over fifty, which represent the most common group of patients. However, prostate cancer remains most often undiscovered, even if determination would be possible. Determination of prostate cancer typically occurs by physical examination or by screening blood tests, such as the PSA (prostate specific antigen) test. When suspected to prostate cancer the cancer is typically confirmed by removing a piece of the prostate (biopsy) and examining it under a microscope. Further tests, such as X-rays and bone scans, may be performed to determine whether prostate cancer has spread.

Treatment of prostate cancer still remains an unsolved challenge. Conventional therapy methods may be applied for treatment of prostate cancer such as surgery, radiation therapy, hormonal therapy, occasionally chemotherapy, proton therapy, or some combination of these. However, the age and underlying health of the man as well as the extent of spread, appearance under the microscope, and response of the cancer to initial treatment are important in determining the outcome of the disease. Since prostate cancer is a disease, typically diagnosed in older men, many will die of other causes before a slowly advancing prostate cancer can spread or cause symptoms. This makes treatment selection difficult. The decision whether or not to treat localized prostate cancer (a tumor that is contained within the prostate) with curative intent is a trade-off between the expected beneficial and harmful effects in terms of patient survival and quality of life.

However, the above therapy methods, such as surgery, radiation therapy, hormonal therapy, and chemotherapy, etc., all suffer from severe limitations. By way of example, surgical removal of the prostate, or prostatectomy, is a common treatment either for early stage prostate cancer or for cancer which has failed to respond to radiation therapy. It may cause nerve damage that significantly alters the quality of life. The most common serious complications are loss of urinary control and impotence. However, even if the prostate cancer could be removed successfully, spread of prostate cancer throughout the organism remains an unsolved problem.

Radiation therapy is commonly used in prostate cancer treatment. It may be used instead of surgery for early cancers, and it may also be used in advanced stages of prostate cancer to treat painful bone metastases. Radiation treatments also can be combined with hormonal therapy for intermediate risk disease, when radiation therapy alone is less likely to cure the cancer. However, radiation therapy also bears high risks and often leads to a complete loss of immune defence due to destruction of the patient's immune system. Furthermore, radiation therapy is typically applied locally at the site of cancer growth and thus may not avoid the above problem of spread of prostate cancer throughout the organism. If applied systemically, radiation therapy may lead to severe damages to cells and immune system.

Chemotherapy was considered as a less effective sort of treatment for prostate cancers since only very few patients even respond to this sort of therapy. However, some patients (responders), having a metastasizing prostate carcinoma, may benefit from chemotherapy. The response rate is at about 20% and chemotherapy will thus play a role during treatment of the tumor relapse and failing of hormonal therapy. However, chemotherapy will typically be only palliative and does not lead to a total elimination of the prostate cancer in the patient. Typical chemotherapeutic agents include cyclophosphamid, doxorubicin, 5-fluoruracil, adriamycin, suramin and other agents, however, none of these resulted in a significant longer survival of the patients. In a more recent study, published 2004 in the New England Journal of Medicine, longer survival of median 2.5 months could be demonstrated for patients which received a dosis of the agent docetaxel every three weeks (Tannock I F et al. Docetaxel plus prednisone or mitoxantrone plus prednisone for advanced prostate cancer. N Engl J Med. 2004 October 7; 351(15):1502-12).

Hormonal therapy typically uses medications or a combination of hormonal therapy with surgery to block prostate cancer cells from getting dihydrotestosterone (DHT), a hormone produced in the prostate and required for the growth and spread of most prostate cancer cells. Blocking DHT often causes prostate cancer to stop growing and even shrink. However, hormonal therapy rarely cures prostate cancer because cancers which initially respond to hormonal therapy typically become resistant after one to two years. E.g. palliative androgen deprivation therapy can induce remissions in up to 80% of the patients, but after 15-20 months, tumor cells become hormone-insensitive and androgen-independent prostate cancer develops. In this situation treatment options are rare, as chemotherapy has been of limited efficacy (see above). Hormonal therapy is therefore usually used when cancer has spread from the prostate. It may also be given to certain men undergoing radiation therapy or surgery to help prevent return of their cancer.

Based on the results of an interim analysis of the phase III trial (ClinicalTrials.gov ID NCT00887198), Abiraterone acetate/prednisone was approved in Europe and the US for the treatment of chemonaive patients with castration refractory prostate cancer since it prolonged progression free survival and other secondary endpoints significantly compared to the placebo/prednisone control. Whereas there was a trend for improved overall survival, the difference was not statistically significant (Ryan C. J. et al. (2013). Abiraterone in metastatic prostate cancer without previous chemotherapy. N Engl J Med. January 10; 368(2):138-48.).

As soon as patients develop symptoms of their disease, the initiation of chemotherapy (preferably with docetaxel) is recommended. Additional therapeutic options in this situation are radiation of painful bone metastases or treatment with bone-seeking radioisotopes like strontium-89 or samarium-153 or radium 223-chloride (alpharadin).

After progression of the disease during treatment with docetaxel, treatment is continued with the new antihormonal agents enzalutamide (Scher H. I. et al. (2012). Increased survival with enzalutamide in prostate cancer after chemotherapy. N Engl J Med. September 27; 367(13):1187-97), abiraterone acetate/prednisone (de Bono, J. S., et al. (2011). Abiraterone and increased survival in metastatic prostate cancer. N. Engl. J. Med. 364:1995-2005), or the new taxane cabazitaxel/prednisone (Yap, et al. (2011). The changing therapeutic landscape of castration-resistant prostate cancer. Nat Rev Clin Oncol. 8:597-610) are further approved treatment options having demonstrated a survival benefit in phase III trials—however none of these treatments is able to induce long-term survival and the chemotherapeutic agents have considerable side effects. New therapeutic options that could prolong the effects of the available treatments without adding major toxicity are urgently required.

As one approach, the above discussed standard therapies used for organ-confined prostate cancer, including radical prostatectomy or radiation therapy such as external (beam) irradiation and brachytherapy may under some circumstances incorporate also neoadjuvant or adjuvant hormonal therapy (see e.g. Totterman, T. H., A. Loskog, et al. (2005). “The immunotherapy of prostate and bladder cancer.” BJU Int 96(5): 728-35.). While these therapies are relatively effective in the short term, a significant proportion (30-40%) of patients having initially localized disease will ultimately relapse. For metastatic prostate cancer the main therapy is androgen ablation. While this usually provides cytoreduction and palliation, progression to hormone-refractory disease typically occurs within 14-20 months. Many clinical studies have been reported in the field of chemotherapy for advanced androgen-independent prostate cancer. Only recently two trials have revealed that chemotherapy marginally improves the overall survival of patients with hormone-refractory (castration-resistant) disease.

Summarizing the above, standard techniques such as the above mentioned surgery, radiation therapy, hormonal therapy, occasionally chemotherapy, proton therapy, etc., if applied alone, do not appear to be suitable for an efficient treatment of prostate cancer (PCa). One improved way of treatment may therefore include such standard techniques, however, in combination with other approaches. According to the present invention, the adaptive immune system is addressed as an approach for the treatment or supplementary treatment of prostate cancer (PCa).

As known in the art, the immune system plays an important role in the treatment and prevention of numerous diseases. According to the present stage of knowledge, various mechanisms are provided by mammalians to protect the organism by identifying and killing, e.g., tumor cells. For the purposes of the present invention, these tumor cells have to be detected and distinguished from the organism's normal (healthy) cells and tissues.

The immune systems of vertebrates such as humans consist of many types of proteins, cells, organs, and tissues, which interact in an elaborate and dynamic network. As part of this complex immune response, the vertebrate system adapts over time to recognize particular pathogens or tumor cells more efficiently. The adaptation process generates immunological memory and allows even more effective protection during future encounters. This process of adaptive or acquired immunity forms the basis for vaccination strategies.

The adaptive immune system is antigen-specific and requires the recognition of specific “self” or “non-self” antigens during a process called antigen presentation. Antigen specificity allows for the generation of responses that are tailored to specific pathogens or pathogen-infected cells or tumor cells. The ability to mount these tailored responses is maintained in the body by so called “memory cells”. When a pathogen infects the body more than once, these specific memory cells are used to quickly eliminate it. The adaptive immune system thus allows for a stronger immune response as well as for an immunological memory, where each pathogen or tumor cell is “remembered” by one or more signature antigens.

The major components of the adaptive immune system in vertebrates predominantly include lymphocytes on the cellular level and antibodies on the molecular level. Lymphocytes as cellular components of the adaptive immune system include B cells and T cells which are derived from hematopoietic stem cells in the bone marrow. B cells are involved in the humoral response, whereas T cells are involved in cell mediated immune response. Both B cells and T cells carry receptor molecules that recognize specific targets. T cells recognize a “non-self” target, such as a pathogenic target structure, only after antigens (e.g. small fragments of a pathogen) have been processed and presented in combination with a “self” receptor called a major histocompatibility complex (MHC) molecule. In contrast, the B cell antigen-specific receptor is an antibody molecule on the B cell surface, and recognizes pathogens as such when antibodies on its surface bind to a specific foreign antigen. This antigen/antibody complex is taken up by the B cell and processed by proteolysis into peptides. The B cell then displays these antigenic peptides on its surface MHC class II molecules. This combination of MHC and antigen attracts a matching helper T cell, which releases lymphokines and activates the B cell. As the activated B cell then begins to divide, its offspring secretes millions of copies of the antibody that recognizes this antigen. These antibodies circulate in blood plasma and lymph, bind to pathogens or tumor cells expressing the antigen and mark them for destruction by complement activation or for uptake and destruction by phagocytes. As a cellular component of the adaptive immune system, cytotoxic T cells (CD8+) may also form a CTL-response. Cytotoxic T cells (CD8+) can recognize peptides from endogenous pathogens and self-antigens bound by MHC type I molecules. CD8+-T cells carry out their killing function by releasing cytotoxic proteins.

Mechanisms of the immune system may thus form targets for curative treatments of various diseases. Appropriate methods are typically based on the administration of adjuvants to elicit an innate immune response or on the administration of antigens or immunogens in order to evoke an adaptive immune response. As antigens are typically based on specific components of pathogens (e.g. surface proteins) or fragments thereof, administration of nucleic acids to the patient which is followed by the expression of desired polypeptides, proteins or antigens is envisaged as well.

Castration-resistant prostate cancer is the only cancer indication so far in which an active immune therapy designed to induce specific immune responses has been approved based on a significant prolongation in overall survival. Sipuleucel-T (Provenge®), an active immunotherapy consisting of autologous antigen presenting cells pulsed with a fusion protein consisting of the prostate cancer associated antigen PAP and the adjuvant GM-CSF, has been shown to prolong survival in a phase III trial enrolling 512 patients by a median of 4.1 months compared to placebo (25.8 vs 21.7 mo; p=0.03) in patients with asymptomatic or minimally symptomatic castration-resistant prostate cancer. Based on these results sipuleucel-T has been approved by the FDA for the treatment of this group of patients in 2010 (Kantoff, P., Higano, C., Shore, N., Berger, E. R., Small, E. J., et al. (2010). Sipuleucel-T immunotherapy for castration-resistant prostate cancer. N. Engl. J. Med. 363:411-422).

As a further example, vaccination studies based on known prostate related antigens have been carried out in Noguchi et al. (2003) and (2004) (see e.g. Noguchi, M., K. Itoh, et al. (2004). “Phase I trial of patient-oriented vaccination in HLA-A2-positive patients with metastatic hormone-refractory prostate cancer.” Cancer Sci 95(1): 77-84; and Noguchi, M., K. Kobayashi, et al. (2003). “Induction of cellular and humoral immune responses to tumor cells and peptides in HLA-A24 positive hormone-refractory prostate cancer patients by peptide vaccination.” Prostate 57(1): 80-92.). Noguchi et al. (2003) and (2004) carried out two phase I studies with a multipeptide trial of vaccination in metastatic hormone-resistant (castration-resistant) prostate cancer patients showing increased cellular as well as humoral immune responses to the selected targets. The vaccination strategy was safe, well tolerated with no major toxic effects. Stabilization or reduction of prostate specific antigen (PSA) levels was also observed and only one patient showed disappearance of a bone metastasis. The main limitation of this approach that makes it difficult for clinical applications consists in the need of a priori knowledge of the patient's HLA haplotype as well as of peptide expression by prostate cancer cells.

Some other recent approaches utilize cell based vaccination strategies, e.g. the use of different antigens in vaccination strategies or the use of dendritic cells loaded with different antigens or fragments thereof. According to one example, vaccination of prostate cancer patients has been tested in clinical trials with autologous dendritic cells pulsed with recombinant human PSA (see e.g. Barrou, B., G. Benoit, et al. (2004). “Vaccination of prostatectomized prostate cancer patients in biochemical relapse, with autologous dendritic cells pulsed with recombinant human PSA.” Cancer Immunol Immunother 53(5): 453-60). As a result of vaccination of advanced prostate cancer patients with PSCA and PSA peptide-loaded dendritic cells, 5 out of 10 patients showed an immune response against at least one antigen (see e.g. Thomas-Kaskel, A. K., R. Zeiser, et al. (2006). “Vaccination of advanced prostate cancer patients with PSCA and PSA peptide-loaded dendritic cells induces DTH responses that correlate with superior overall survival.” Int J Cancer 119(10): 2428-34.).

In another example, Murphy et al. (1996) carried out vaccination of prostate cancer patients in a corresponding phase I trial with two HLA-A*0201 PSMA epitopes to compare vaccination based on the peptide alone with vaccination based on pulsed DCs. The results showed that more patients responded to the vaccination, if the patients were vaccinated with pulsed DCs. This study showed that vaccination based on DCs loaded with peptides or proteins leads at least for a number of instances to detectable immune responses as well as a temporary PSC decline or stabilization (see e.g. Murphy, G., B. Tjoa, et al. (1996). “Phase I clinical trial: T-cell therapy for prostate cancer using autologous dendritic cells pulsed with HLA-A0201-specific peptides from prostate-specific membrane antigen.” Prostate 29(6): 371-80).

Vaccination of prostate cancer patients may also be carried out with combinations of peptides loaded on dendritic cells, e.g. with peptide cocktail-loaded dendritic cells (see e.g. Fuessel, S., A. Meye, et al. (2006). “Vaccination of hormone-refractory prostate cancer patients with peptide cocktail-loaded dendritic cells: results of a phase I clinical trial.” Prostate 66(8): 811-21). The cocktail contained peptides from PSA, PSMA, Survivin, Prostein and Trp-p8 (transient receptor potential p8). Clinical trials were also carried out with a dendritic cell-based multi-epitope immunotherapy of hormone-refractory prostate carcinoma (see e.g. Waeckerle-Men, Y., E. Uetz-von Allmen, et al. (2006). “Dendritic cell-based multi-epitope immunotherapy of hormone-refractory prostate carcinoma.” Cancer Immunol Immunother 55(12): 1524-33). Waeckerle-Men, Y., E. Uetz-von Allmen, et al. (2006) tested vaccination of hormone-refractory prostate carcinoma with peptides from PSCA, PAP (prostatic acid phosphatase), PSMA and PSA.

While vaccination with antigenic proteins or peptides, e.g. when loaded on dendritic cells, is a common method for eliciting an immune response, immunization or vaccination may also be based on the use of nucleic acids in order to incorporate the required genetic information into the cell. In general, various methods have been developed for introducing nucleic acids into cells, such as calcium phosphate transfection, polyprene transfection, protoplast fusion, electroporation, microinjection and lipofection, with lipofection having been in particular proven to be a suitable method.

Vaccination treatment of prostate cancer may, e.g., be based on the transfection of total mRNA derived from the autologous tumor into DCs (see Heiser et al. (2002) (see e.g. Heiser, A., D. Coleman, et al. (2002). “Autologous dendritic cells transfected with prostate-specific antigen RNA stimulate CTL responses against metastatic prostate tumors.” J Clin Invest 109(3): 409-17.). This strategy has the advantage of targeting multiple HLA class I and class II patient specific tumor associated antigens (TAAs) without prior HLA typing. Moreover, even stromal antigens were targeted by this strategy, since mRNA was obtained from surgical samples and not from tumor cell lines. As an example, Heiser et al. developed a DC-based immunotherapy protocol in which DCs were transfected with mRNA encoding PSA. The vaccination was well tolerated and induced an increased T cell response to PSA. However, such DC-based anti-prostate cancer vaccines appear to generate a strong T cell response, which may be accompanied by clinical response though the frequency of the latter still remains unsatisfactory.

DNA may also be utilized as a nucleic acid in vaccination strategies in order to incorporate the required genetic information into the cell. E.g., DNA viruses may be used as a DNA vehicle. Because of their infectious properties, such viruses achieve a very high transfection rate. The viruses used are genetically modified in such a manner that no functional infectious particles are formed in the transfected cell. E.g., phase I trials were carried out in a study of Eder et al. (2000) using recombinant vaccinia viruses expressing PSA. The authors demonstrated T cell immune responses to PSA and also serum PSA stabilizations in selected patients. (see e.g. Eder, J. P., P. W. Kantoff, et al. (2000). “A phase I trial of a recombinant vaccinia virus expressing prostate-specific antigen in advanced prostate cancer.” Clin Cancer Res 6(5): 1632-8.). The inflammatory response triggered by the highly immunogenic peptides from the recombinant virus may enhance the immunogenicity of the foreign protein. It was shown, however, that the immune system reduces the replication of the recombinant virus and thereby limits the clinical outcome. Even though recombinant vaccines have shown immunogenicity and evidence for a tumor response was shown in several trials, these results need to be further substantiated for further clinical testing.

According to a further approach, vaccination of hormone-refractory prostate cancer patients was carried out with DNA plasmids expressing PSA (see e.g. Pavlenko, M., A. K. Roos, et al. (2004). “A phase I trial of DNA vaccination with a plasmid expressing prostate-specific antigen in patients with hormone-refractory prostate cancer.” Br J Cancer 91(4): 688-94). Garcia-Hernandez et al. (2007) showed that therapeutic and prophylactic vaccination with a plasmid or a virus-like replicon coding for STEAP (Six Transmembrane Epithelial Antigen of the Prostate) prolonged the survival in tumor-challenged mice (see e.g. Garcia-Hernandez Mde, L., A. Gray, et al. (2007). “In vivo effects of vaccination with six-transmembrane epithelial antigen of the prostate: a candidate antigen for treating prostate cancer.” Cancer Res 67(3): 1344-51). Recently STEAP was identified as indicator protein for advanced human prostate cancer, which is highly overexpressed in human prostate cancer. Its function is currently unknown.

While using DNA as a carrier of genetic information, it is, however, not possible to rule out the risk of uncontrolled propagation of the introduced gene or of viral genes, for example due to potential recombination events. This also entails the risk of the DNA being inserted into an intact gene of the host cell's genome by e.g. recombination, with the consequence that this gene may be mutated and thus completely or partially inactivated or the gene may give rise to misinformation. In other words, synthesis of a gene product which is vital to the cell may be completely suppressed or alternatively a modified or incorrect gene product is expressed. The DNA may e.g. be integrated into a gene which is involved in the regulation of cell growth. In this case, the host cell may become degenerate and lead to cancer or tumor formation. Furthermore, if the DNA introduced into the cell is to be expressed, it is necessary for the corresponding DNA vehicle to contain a strong promoter, such as the viral CMV promoter. The integration of such promoters into the genome of the treated cell may result in undesired alterations of the regulation of gene expression in the cell. Another risk of using DNA as an agent to induce an immune response (e.g. as a vaccine) is the induction of pathogenic anti-DNA antibodies in the treated patient thereby eliciting a (possibly fatal) immune response.

Thus, in order to effectively stimulate the immune system to allow treatment of prostate cancer (PCa) while avoiding the problems of uncontrolled propagation of an introduced gene due to DNA based compositions, RNA based antigen compositions have been developed. WO 2009/046975 provides a composition comprising at least one RNA encoding at least two, three or four (preferably different) antigens selected from the group consisting of PSA (Prostate-Specific Antigen; also known as KLK3 or Kallikrein-3), PSMA (Prostate-Specific Membrane Antigen), PSCA (Prostate Stem Cell Antigen) and STEAP (Six Transmembrane Epithelial Antigen of the Prostate).

Even though the combination of antigens in the composition mentioned above increases considerably the number of responders amongst the group of prostate cancer patients, there are nevertheless non-responding subjects, who do not benefit from any of the approaches known in the art. Given the high incidence and the increased mortality rate in prostate cancer, there is thus a strong need for further, alternative or improved treatment protocols.

It is thus an object of the present invention to provide a composition for treatment of prostate cancer (PCa) or a vaccine by stimulating the immune system.

The object underlying the present invention is solved by the claimed subject matter.

### SUMMARY OF THE INVENTION

This object is solved by the subject matter of the present invention, particularly by a composition comprising at least one mRNA, wherein the at least one mRNA encodes the following antigens:
STEAP (Six Transmembrane Epithelial Antigen of the Prostate);
PSA (Prostate-Specific Antigen),
PSMA (Prostate-Specific Membrane Antigen),
PSCA (Prostate Stem Cell Antigen);
PAP (Prostatic Acid Phosphatase), and
MUC1 (Mucin 1),
   or fragments or variants thereof.

Surprisingly, it has been found that the specific combination of the antigens, antigenic proteins or antigenic peptides of the afore mentioned group encoded by at least one mRNA as contained in a composition according to the present invention, is capable to effectively stimulate the (adaptive) immune system to allow treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto. The advantageous effects on the treatment of the diseases mentioned above are achieved irrespective of whether the combination of antigens according to the invention is applied as one single composition or by separate administration of the individual antigens. Accordingly, any combination of antigens described herein, e.g. in the form of six separate mRNA formulations, may fulfil the very same purposes and achieves the desired effect. The number of responders to such a vaccination strategy is expected to be significantly increased as compared to other approaches. Herein, the terms antigens, antigenic proteins or antigenic peptides may be used synomously. In the context of the present invention, an inventive composition shall be further understood as a composition, which is able to elicit an immune response, preferably an adaptive immune response as defined herein, due to at least one of the component(s) contained in the composition or, rather, due to at least one of the antigens encoded by the at least one component of the composition, i.e. by the at least one mRNA encoding the antigens as defined above. According to the invention, the combination of antigens, whether administered separately (e.g. concurrently) or as one single composition, is capable of eliciting the desired immune response. Separate administration may mean that the distinct mRNAs are either essentially simultaneously, e.g. within 10 minutes or time-staggered over an extended period of time, e.g. more than 30 minutes.

In the following, the combination of antigens according to the invention will be illustrated by the description of a composition comprising at least one mRNA encoding the combination of antigens. It is understood that the at least one mRNA according to the invention is characterized by the features as described herein, irrespective of whether it is administered as one single composition or in the form of separate formulations, e.g. formulated as six separate mRNAs, each of which encode one antigen and which are administered separately (e.g. concurrently).

Among the large number of overexpressed antigens in prostate cancer cells, the present invention did specifically select PSA, PSMA, PSCA, STEAP, PAP and MUC1. These antigens were identified according to the invention to represent possible targets of immunotherapy. According to the invention, one or more of the above antigens are encoded by the at least one ORF/coding region/coding sequence provided by the at least one mRNA. In this context, a messenger RNA is typically a single-stranded RNA, which is composed of (at least) several structural elements, e.g. an optional 5′ UTR region, an optional upstream positioned ribosomal binding site followed by a coding region, an optional 3′ UTR region, which may be followed by a poly-A tail (and/or a poly-C tail). According to the invention, the composition comprises at least on mRNA, which endodes at least the six antigens defined above. Therein, one mRNA may encode one or more antigens as long as the composition as such provides the at least six antigens as defined above. The at least one mRNA of the composition may thus comprise more than one ORF/coding region/coding sequence, wherein the composition as a whole comprises at least one coding region for each of the at least six antigens as defined above. Alternatively, a coding region for each of the at least six antigens may be located on separate mRNAs of the composition. More preferred embodiments for the at least one mRNA are provided below:

One of the antigens encoded by the at least one mRNA of the composition is PSA. In the context of this invention, “PSA” is “Prostate-specific antigen” and may be synomously named KLK3 (Kallikrein-3) in the literature. Prostate-specific antigen (PSA) is a 33 kDa protein and an androgen-regulated kallikrein-like, serine protease that is produced exclusively by the epithelial cells of all types of prostatic tissue, benign and malignant. Particularly, PSA is highly expressed by normal prostatic epithelial cells and represents one of the best characterized tumor associated antigens in prostate cancer. Physiologically, it is present in the seminal fluid at high concentration and functions to cleave the high molecular weight protein responsible for the seminal coagulum into smaller polypeptides. This action results in liquefaction of the coagulum. PSA is also present in the serum and can be measured reliably by either a monoclonal immunoradiometric assay or a polyclonal radioimmunoassay. PSA is the most widely used tumor marker for screening, diagnosing, and monitoring prostate cancer today. In particular, several immunoassays for the detection of serum PSA are in widespread clinical use. Recently, a reverse transcriptase-polymerase chain reaction (RT-PCR) assay for PSA mRNA in serum has been developed.

In the context of this invention, the preferred sequence of the at least one mRNA encoding PSA (prostate specific antigen) may contain a sequence coding for the amino acid sequence of PSA as deposited under accession number NP_001639.1 (FIG. 31; SEQ ID NO: 76) or a sequence as deposited under accession number NM_001648. Preferably, the at least one mRNA contains a coding sequence as shown in any of FIG. 2, 3 or 27 (SEQ ID NOs: 2, 3 or 82). More preferably, the at least on mRNA contains or consists of a sequence as shown in FIG. 1 or 19 (SEQ ID NO: 1 or 19). According to a further preferred embodiment, the at least one mRNA of the composition may alternatively encode a PSA antigen sequence selected from a fragment, a variant or an epitope of a PSA sequence as deposited under accession number NP_001639.1 or as shown in FIG. 31 (SEQ ID NO: 76) or may contain a fragment or variant of the sequence as deposited under accession number NM_001648 or as shown in any of FIG. 1, 2, 3, 19 or 27 (SEQ ID NOs: 1, 2, 3, 19 or 82).

PSMA is another antigen, which is encoded by the at least one mRNA of the composition. In the context of this invention “PSMA” is “Prostate-specific membrane antigen” and may be synomously named FOLH1 (Folate hydrolase 1) or “PSM”. PSMA is a 100 kDa type II transmembrane glycoprotein, wherein PSMA expression is largely restricted to prostate tissues, but detectable levels of PSMA mRNA have been observed in brain, salivary gland, small intestine, and renal cell carcinoma (Israeli et al., 1993, Cancer Res 53: 227-230). PSMA is highly expressed in most primary and metastatic prostate cancers, but is also expressed in most normal intraepithelial neoplasia specimens (Gao et al. (1997), supra). Particularly, PSMA is highly expressed in prostate cancer cells and nonprostatic solid tumor neovasculature and is a target for anticancer imaging and therapeutic agents. PSMA acts as a glutamate carboxypeptidase (GCPII) on small molecule substrates, including folate, the anticancer drug methotrexate, and the neuropeptide N-acetyl-L-aspartyl-L-glutamate. In prostate cancer, PSMA expression has been shown to correlate with disease progression, with highest levels expressed in hormone-refractory and metastatic disease. The cellular localization of PSMA is cytoplasmic and/or membranous. PSMA is considered a biomarker for prostate cancer (PCa) and is under intense investigation for use as an imaging and therapeutic target.

In the context of this invention the preferred sequence of the at least one mRNA encoding PSMA (prostate specific membrane antigen) may contain a sequence coding for the amino acid sequence of PSMA as deposited under accession number NP_004467.1 (FIG. 32; SEQ ID NO: 77) or a sequence as deposited under accession number NM_004476. Preferably, it contains a coding sequence as shown in any of FIG. 5, 6 or 28 (SEQ ID NO: 5, 6 or 83). More preferably, the at least one mRNA contains or consists of a sequence as shown in FIG. 4 or 20 (SEQ ID NO: 4 or 20). According to a further preferred embodiment, the at least one mRNA of the composition may alternatively encode a PSMA antigen sequence selected from a fragment, a variant or an epitope of a PSMA sequence as deposited under accession number NP_004467.1 or as shown in FIG. 32 (SEQ ID NO: 77) or may contain a fragment or variant of the sequence as deposited under accession number NM_004476 or as shown in any of FIG. 4, 5, 6, 20 or 28 (SEQ ID NOs: 4, 5, 6, 20 or 83).

A further antigen encoded by the at least one mRNA of the composition according to the invention is PSCA. In the context of this invention “PSCA” is “prostate stem cell antigen”. PSCA is widely over-expressed across all stages of prostate cancer, including high grade prostatic intraepithelial neoplasia (PIN), androgen-dependent and androgen-independent prostate tumors. The PSCA gene shows 30% homology to stem cell antigen-2, a member of the Thy-I/Ly-6 family of glycosylphosphatidylinositol (GPI)-anchored cell surface antigens, and encodes a 123 amino acid protein with an amino-terminal signal sequence, a carboxy-terminal GPI-anchoring sequence, and multiple N-glycosylation sites. PSCA mRNA expression is highly upregulated in both androgen dependent and androgen independent prostate cancer xenografts. In situ mRNA analysis localizes PSCA expression to the basal cell epithelium, the putative stem cell compartment of the prostate. Flow cytometric analysis demonstrates that PSCA is expressed predominantly on the cell surface and is anchored by a GPI linkage. Fluorescent in situ hybridization analysis localizes the PSCA gene to chromosome 8q24. 2, a region of allelic gain in more than 80% of prostate cancers. PSCA may be used as a prostate cancer marker to discriminate between malignant prostate cancers, normal prostate glands and non-malignant neoplasias. For example, PSCA is expressed at very high levels in prostate cancer in relation to benign prostatic hyperplasia (BPH).

In the context of this invention, the preferred sequence of the at least one mRNA encoding PSCA (prostate stem cell antigen) may contain a sequence coding for the amino acid sequence of PSCA as deposited under accession number 043653.1 (FIG. 33; SEQ ID NO: 78) or a sequence as deposited under accession number NM_005672. Preferably, it contains a coding sequence as shown in any of FIG. 8, 9 or 29 (SEQ ID NOs: 8, 9 or 84). More preferably, the at least one mRNA comprises or consists of a sequence as shown in FIG. 7 or 21 (SEQ ID NO: 7 or 21). According to a further preferred embodiment, the at least one mRNA of the composition may alternatively encode a PSCA antigen sequence selected from a fragment, a variant or an epitope of a PSCA sequence as deposited under accession number 043653.1 or as shown in FIG. 33 (SEQ ID NO: 78) or may contain a fragment or variant of the sequence as deposited under accession number NM_005672 or as shown in any of FIG. 7, 8, 9, 21 or 29 (SEQ ID NOs: 7, 8, 9, 21 or 84).

In addition, STEAP is encoded by the at least one mRNA of the composition according to the invention. In the context of this invention, “STEAP” is “six transmembrane epithelial antigen of the prostate” and may synomously be named STEAP1. STEAP or STEAP-1 is a novel cell surface protein and is expressed predominantly in human prostate tissue and in other common malignancies including prostate, bladder, colon, and ovarian carcinomas, and in Ewing's sarcoma, suggesting that it could function as an almost universal tumor antigen. Particularly, STEAP is highly expressed in primary prostate cancer, with restricted expression in normal tissues. STEAP positivity in bone marrow samples was highly correlated with survival with new metastasis in Kaplan Meier analysis (p=0.001).

In the context of this invention, the preferred sequence of the at least one mRNA encoding STEAP (six transmembrane epithelial antigen of the prostate) (or STEAP1) may contain a sequence coding for the amino acid sequence of STEAP as deposited under accession number NP_036581.1 (FIG. 34; SEQ ID NO: 79) or a sequence as deposited under accession number NM_012449. Preferably, it contains a coding sequence as shown in any of FIG. 11, 12 or 30 (SEQ ID NO: 11, 12 or 85). More preferably, the at least one mRNA contains or consists of a sequence as shown in FIG. 10 or 22 (SEQ ID NO: 10 or 22). According to a further preferred embodiment, the at least one mRNA of the composition may alternatively encode a STEAP antigen sequence selected from a fragment, a variant or an epitope of a STEAP sequence as deposited under accession number NP_036581.1 or as shown in FIG. 34 (SEQ ID NO: 79) or may contain a fragment or variant of the sequence as deposited under accession number NM_012449 or as shown in any of FIG. 10, 11, 12, 22, or 30 (SEQ ID NOs: 10, 11, 12, 22 or 85).

Furthermore, the at least one mRNA of the composition according to the invention encodes PAP. “PAP” is “prostatic acid phosphatase” and may be synonymously referred to as, for instance, PSAP (prostate specific acid phosphatase) or ACPP (acid phosphatase, prostate). PAP is an enzyme, which is secreted by epithelial cells of the prostate gland and catalyzes the conversion of orthophosphoric monoester to alcohol and orthophosphate. >95% of normal adult prostate tissue samples, including normal tissue adjacent to tumor, as well as >95% of primary adenocarcinomas, strongly express PAP. PAP expression can be detected in some normal human tissues besides the prostate (e.g. kidney, lung, testis, colon, pancreas) but at a level approximately 1-2 orders of magnitude less, and PAP has generally been considered a tissue-specific prostate antigen, highly expressed in both normal and malignant prostate cells. Furthermore it has been demonstrated that PAP is strongly expressed in prostate cancer bone metastases and may play a causal role in the osteoblastic phase of the disease. PAP has been shown to induce long term CD4+ and CD8+ T-cell responses, including CTL responses, in patients. Treatment of prostate cancer patients with autologous antigen presenting cells stimulated with PAP has resulted in improved survival and a favorable safety profile.

In the context of this invention, the preferred sequence of the at least one mRNA encoding PAP may contain a sequence coding for the amino acid sequence of PAP as deposited under accession number NP_001090.2 (FIG. 35; SEQ ID NO: 80) or a sequence as deposited under accession number NM_001099.4. Preferably, it contains a coding sequence as shown in any of FIG. 14 or 15 (SEQ ID NO: 14 or 15). More preferably, the at least one mRNA comprises or consists of a sequence as shown in FIG. 13 or 23 (SEQ ID NO: 13 or 23). According to a further preferred embodiment, the at least one mRNA of the composition may alternatively encode a PAP antigen sequence selected from a fragment, a variant or an epitope of a PAP sequence as deposited under accession number NP_001090.2 or as shown in FIG. 35 (SEQ ID NO: 80) or may contain a fragment or variant of the sequence as deposited under accession number NM_001099.4 or as shown in FIG. 13, 14, 15 or 23 (SEQ ID NO: 13, 14, 15 or 23).

Finally, MUC1 is encoded by the at least one mRNA of the composition according to the invention. “MUC1” is “Mucin 1” and may be synonymously referred to as, for instance, CD227 or DF3. MUC1 is a large mucinous glycoprotein that is normally expressed on the luminar surface of glandular epithelia. Its function in normal epithelia is to lubricate and to protect epithelial cells. The expression of MUC1 is often increased, no longer restricted to a luminal surface and characterized by aberrant glycosylation in many human malignancies, including prostate cancer. MUC1 is expressed in about 60% of primary prostate cancers and 90% of lymph node metastases. In addition, 86% of MUC1-positive primary prostate tumors were Gleason grade ≧7, supporting an association with more aggressive disease. Gene expression profiling of human prostate cancers has also shown that MUC1 is highly expressed in subgroups with aggressive clinicopathologic features and an elevated risk of recurrence. Both over- and underexpression of MUC-1 have been found to increase the risk of prostate cancer progression. MUC1 has been shown to be immunogenic and has been described to induce specific immune responses comprising CD8+ CTLs and IgM antibodies in patients. Vaccination against MUC1 using different vaccination approaches was associated with trends for clinical benefit in phase II trials in patients with advanced non-small cell lung cancer and appeared well tolerated. Vaccination against MUC1 in prostate cancer using the same vaccines was associated with a prolongation in PSA doubling time in some patients. (Bilusic, M., et al. (2011) Immunotherapy in prostate cancer: emerging strategies against a formidable foe. Vaccine. 29:6485-6497; Dreicer, R. et al. (2009). MVA-MUC1-IL2 vaccine immunotherapy (TG4010) improves PSA doubling time in patients with prostate cancer with biochemical failure. Invest New Drugs. 27:379-386, Sangha, R. and North, S. (2007). L-BLP25: a MUC1-targeted peptide vaccine therapy in prostate cancer. Expert Opin Biol Ther. 7:1723-1730).

In the context of the present invention, the preferred sequence of the at least one mRNA, preferably of the mRNA, encoding MUC1 may contain a sequence coding for the amino acid sequence of MUC1 as deposited under accession number AAA60019.1 (FIG. 36; SEQ ID NO: 81), or a truncated amino acid sequence as shown in FIG. 37 (SEQ ID NO: 86; MUC1 5×VNTR) or the at least one mRNA may contain a sequence as deposited under accession number J05582.1. Preferably, it contains a coding sequence as shown in any of FIG. 17, 18 or 38 (SEQ ID NO: 17, 18 or 87). More preferably, the at least one mRNA contains or consists of a sequence as shown in FIG. 16 or 24 (SEQ ID NO: 16 or 24). According to a further preferred embodiment, the at least one mRNA of the composition may alternatively or additionally encode a MUC1 antigen sequence selected from a fragment, a variant or an epitope of a MUC1 sequence as deposited under accession number AAA60019.1 or as shown in FIG. 36 or 37 (SEQ ID NO: 81 or 86) or may contain a fragment or variant of the sequence as deposited under accession number J05582.1 or as shown in FIG. 16, 17, 18, 24 or 38 (SEQ ID NO: 16, 17, 18, 24 or 87).

Where, in the context of the present invention, reference is made to antigens or fragments or variants thereof, it is understood that the reference concerns the antigen or peptide encoded by one or more of the mRNA sequences provided in the present invention. Further, antigens, antigenic proteins or antigenic peptides as defined above which are encoded by the at least one mRNA of the composition according to the present invention, may comprise fragments or variants of those sequences. Such fragments or variants may typically comprise a sequence having a sequence homology with one of the above mentioned antigens, antigenic proteins or antigenic peptides or sequences or their encoding nucleic acid sequences of at least 5%, 10%, 20%, 30%, 40%, 50%, 60%, preferably at least 70%, more preferably at least 80%, equally more preferably at least 85%, even more preferably at least 90% and most preferably at least 95% or even 97%, to the entire wild-type sequence, either on nucleic acid level or on amino acid level.

“Fragments” of antigens, antigenic proteins or antigenic peptides in the context of the present invention may comprise a sequence of an antigen, antigenic protein or antigenic peptide as defined above, which is, with regard to its amino acid sequence (or its encoded nucleic acid sequence), N-terminally, C-terminally and/or intrasequentially truncated compared to the amino acid sequence of the original (native) protein (or its encoded nucleic acid sequence). Such truncation may thus occur either on the amino acid level or correspondingly on the nucleic acid level. A sequence homology with respect to such a fragment as defined above may therefore preferably refer to the entire antigen, antigenic protein or antigenic peptide as defined above or to the entire (coding) nucleic acid sequence of such an antigen, antigenic protein or antigenic peptide.

Fragments of antigens, antigenic proteins or antigenic peptides in the context of the present invention may furthermore comprise a sequence of an antigen, antigenic protein or antigenic peptide as defined above, which has a length of about 6 to about 20 or even more amino acids, e.g. fragments as processed and presented by MHC class I molecules, preferably having a length of about 8 to about 10 amino acids, e.g. 8, 9, or 10, (or even 6, 7, 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule, i.e. the fragments are typically not recognized in their native form.

Fragments of antigens, antigenic proteins or antigenic peptides as defined herein may also comprise epitopes of those antigens, antigenic proteins or antigenic peptides. Epitopes (also called “antigen determinants”) in the context of the present invention are typically fragments located on the outer surface of (native) antigens, antigenic proteins or antigenic peptides as defined herein, preferably having 5 to 15 amino acids, more preferably having 5 to 12 amino acids, even more preferably having 6 to 9 amino acids, which may be recognized by antibodies or B-cell receptors, i.e. in their native form. Such epitopes of antigens, antigenic proteins or antigenic peptides may furthermore be selected from any of the herein mentioned variants of such antigens, antigenic proteins or antigenic peptides. In this context antigenic determinants can be conformational or discontinous epitopes which are composed of segments of the antigens, antigenic proteins or antigenic peptides as defined herein that are discontinuous in the amino acid sequence of the antigens, antigenic proteins or antigenic peptides as defined herein but are brought together in the three-dimensional structure or continuous or linear epitopes which are composed of a single polypeptide chain. Therefore in this context it is particularly preferred that the fragment of the antigen, the antigenic protein or antigenic peptide comprises at least one epitope of the antigen.

“Variants” of antigens, antigenic proteins or antigenic peptides as defined above may be encoded by the at least one mRNA of the composition according to the present invention, wherein nucleic acids of the at least one mRNA, encoding the antigen, antigenic protein or antigenic peptide as defined above, are exchanged. Thereby, an antigen, antigenic protein or antigenic peptide may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native antigen or antigenic protein, e.g. its specific antigenic property.

The at least one mRNA of the composition according to the present invention may also encode an antigen or an antigenic protein as defined above, wherein the encoded amino acid sequence comprises conservative amino acid substitution(s) compared to its physiological sequence. Those encoded amino acid sequences as well as their encoding nucleotide sequences in particular fall under the term variants as defined above. Substitutions in which amino acids which originate from the same class are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

Furthermore, variants of antigens, antigenic proteins or antigenic peptides as defined above, which may be encoded by the at least one mRNA of the composition according to the present invention, may also comprise those sequences, wherein nucleic acids of the at least one mRNA are exchanged according to the degeneration of the genetic code, without leading to an alteration of respective amino acid sequence of the antigen, antigenic protein or antigenic peptide, i.e. the amino acid sequence or at least part thereof may not differ from the original sequence in one or more mutation(s) within the above meaning.

Furthermore, variants of antigens, antigenic proteins or antigenic peptides as defined above, which may be encoded by the at least one mRNA of the composition according to the present invention, may also comprise those DNA sequences, which correspond to an RNA sequence as defined herewithin and comprise further RNA sequences, which correspond to DNA sequences as defined herewithin. Those skilled in the art are familiar with the translation of an RNA sequence into a DNA sequence (or vice versa) or with the creation of the complementary strand sequence (i.e. by substitution of U residues with T residues and/or by constructing the complementary strand with respect to a given sequence).

In order to determine the percentage to which two sequences (nucleic acid sequences, e.g. RNA or mRNA sequences as defined herein, or amino acid sequences, preferably their encoded amino acid sequences, e.g. the amino acid sequences of the antigens, antigenic proteins or antigenic peptides as defined above) are identical, the sequences can be aligned in order to be subsequently compared to one another. Therefore, e.g. gaps can be inserted into the sequence of the first sequence and the component at the corresponding position of the second sequence can be compared. If a position in the first sequence is occupied by the same component as is the case at a position in the second sequence, the two sequences are identical at this position. The percentage to which two sequences are identical is a function of the number of identical positions divided by the total number of positions. The percentage to which two sequences are identical can be determined using a mathematical algorithm. A preferred, but not limiting, example of a mathematical algorithm which can be used is the algorithm of Karlin et al. (1993), PNAS USA, 90:5873-5877 or Altschul et al. (1997), Nucleic Acids Res., 25:3389-3402. Such an algorithm is integrated in the BLAST program. Sequences which are identical to the sequences of the present invention to a certain extent can be identified by this program.

As used herein, the term “composition” refers to at least one mRNA and, optionally, further excipients. The term “composition” thus comprises any mixture of mRNAs (mRNA species) encoding the antigens as defined above, irrespective of whether the mRNAs are mono-, bi- or multicistronic. Within the meaning of the present invention, the term “composition” also refers to an embodiment consisting of a multicistronic RNA, which encodes all six antigens as defined above. Preferably, the composition contains at least six distinct mRNA species, whereby each mRNA species encodes one of the above antigens. The term “composition” preferably relates to the at least one mRNA together with at least one other suitable substance. In general, the composition may be a pharmaceutical composition, which is designed for use in the medical field. Accordingly, the composition typically comprises at least one further excipient, which is pharmaceutically acceptable and which may be selected, for example, from carriers, vehicles and the like. The “composition” may be a liquid or a dry composition. If the composition is liquid, it will be preferably an aqueous solution or dispersion of the at least one RNA. If the “composition” is a dry composition, it will typically be a lyophilized composition of at least one mRNA. The term “composition”, as used herewithin, further refers to the at least one mRNA of the invention in combination with a further active ingredient. Preferably, the composition is an immunostimulatory composition, i.e. a composition comprising at least one component, which is able to induce an immune response or from which a component, which is able to induce an immune response, is derivable. In this context, the immune response may be the result of the adaptive and/or of the innate immune system.

The composition according to the present invention comprises at least one mRNA encoding at least six antigens as defined above, as it was found out that the specific combination of said antigens is capable of effectively stimulating the (adaptive) immune system, thus allowing treatment of prostate cancer (PCa).

In summary, the object of the present invention is solved by the provision of a composition comprising at least one mRNA coding for a novel combination of antigens as defined herein. In a preferred embodiment, the composition comprises six antigens (PSA, PSMA, PSCA, STEAP, PAP and MUC1) which are encoded by six monocistronic mRNAs, each of these mRNAs encoding a different antigen selected from the defined group of antigens. Alternatively, the composition may comprise a combination of monocistronic, bi- and/or multicistronic mRNAs, wherein more than one of the six antigens is encoded by a bi- or multicistronic mRNA. According to the invention, any combination of mono-, bi- or multicistronic mRNA is envisaged that encode all six antigens as defined herein, e.g. three bicistronic mRNAs, each of which encodes two of the above six antigens or two bicistronic and two monocistronic mRNAs.

According to a preferred embodiment, the composition comprises at least one mRNA, which comprises at least one coding sequence selected from mRNA sequences being identical or at least 80% identical to the RNA sequence of SEQ ID NOs: 2, 5, 8, 11, 14 or 17 (or 87). Even more preferably, the composition comprises six mRNAs, wherein the coding sequence in each mRNA is identical or at least 80% identical to one of the RNA sequences according to SEQ ID NOs: 2, 5, 8, 11, 14 and 17 (or 87).

In a preferred embodiment, each of the at least six antigens of the composition of the present invention, may be encoded by one (monocistronic) mRNA. In other words, the composition of the present invention may contain six (monocistronic) mRNAs, wherein each of these six (monocistronic) mRNAs, may encode just one antigen as defined above.

In a more preferred embodiment, the composition comprises six mRNAs, wherein one mRNA encodes PSA, one mRNA encodes PSMA, one mRNA encodes PSCA, one mRNA encodes STEAP, one mRNA encodes PAP and one mRNA encodes MUC1 or fragments or variants thereof, respectively.

In an even more preferred embodiment, the composition comprises six mRNAs, wherein one mRNA encodes PSA and comprises a coding sequence identical or at least 80% identical to SEQ ID NO: 2, one mRNA encodes PSMA and comprises a coding sequence identical or at least 80% identical to SEQ ID NO: 5, one mRNA encodes PSCA and comprises a coding sequence identical or at least 80% identical to SEQ ID NO: 8, one mRNA encodes STEAP and comprises a coding sequence identical or at least 80% identical to SEQ ID NO: 11, one mRNA encodes PAP and comprises a coding sequence identical or at least 80% identical to SEQ ID NO: 14 and one mRNA encodes MUC1 and comprises a coding sequence identical or at least 80% identical to SEQ ID NO:17 (or 87) (or fragments or variants of each of these sequences) and optionally further excipients.

In an even more preferred embodiment, the composition comprises six mRNAs, wherein one mRNA encodes PSA and comprises the coding sequence according to SEQ ID NO: 2, one mRNA encodes PSMA and comprises the coding sequence according to SEQ ID NO: 5, one mRNA encodes PSCA and comprises the coding sequence according to SEQ ID NO: 8, one mRNA encodes STEAP and comprises the coding sequence according to SEQ ID NO: 11, one mRNA encodes PAP and comprises the coding sequence according to SEQ ID NO: 14 and one mRNA encodes MUC1 and comprises the coding sequence according to SEQ ID NO:17 (or 87) or fragments thereof, respectively.

According to the invention, the at least one mRNA of the composition may preferably comprise a histone stem-loop in the 3′ UTR region. Preferably, the composition comprises six mRNAs, wherein each mRNA comprises a histone stem-loop as defined herein.

According to another particularly preferred embodiment, the composition of the present invention, may comprise (at least) one bi- or even multicistronic mRNA, i.e. (at least) one mRNA which carries the coding sequences of two or more of the six antigens according to the invention. Such coding sequences of two or more antigens of the (at least) one bi- or even multicistronic mRNA may be separated by at least one IRES (internal ribosomal entry site) sequence, as defined below. Thus, the term “encoding two or more antigens” may mean, without being limited thereto, that the (at least) one (bi- or even multicistronic) mRNA may encode e.g. at least two, three, four, five or six (preferably different) antigens of the above mentioned antigens or their fragments or variants within the above definitions. More preferably, without being limited thereto, the (at least) one (bi- or even multicistronic) mRNA may encode e.g. at least two, three, four, five or six (preferably different) antigens of the above mentioned antigens or their fragments or variants within the above definitions. In this context, a so-called IRES (internal ribosomal entry site) sequence as defined above can function as a sole ribosome binding site, but it can also serve to provide a bi- or even multicistronic mRNA as defined above which codes for several proteins, which are to be translated by the ribosomes independently of one another. Examples of IRES sequences which can be used according to the invention are those from picornaviruses (e.g. FMDV), pestiviruses (CFFV), polioviruses (PV), encephalomyocarditis viruses (ECMV), foot and mouth disease viruses (FMDV), hepatitis C viruses (HCV), classical swine fever viruses (CSFV), mouse leukoma virus (MLV), simian immunodeficiency viruses (SIV) or cricket paralysis viruses (CrPV).

According to a further particularly preferred embodiment, the composition of the present invention, may comprise a mixture of at least one monocistronic mRNA as defined above, and at least one bi- or even multicistronic mRNA as defined above. The at least one monocistronic mRNA and/or the at least one bi- or even multicistronic mRNA preferably encode different antigens or their fragments or variants within the above definitions. However, the at least one monocistronic mRNA and the at least one bi- or even multicistronic mRNA may preferably also encode (in part) identical antigens selected from the above mentioned antigens, provided that the composition of the present invention as a whole provides the six antigens as defined above. By providing multiple copies of one or more of the antigens, the relative protein amounts of said one or more antigens can be increased, i.e. the ratio between the amounts of each of the six antigens can be modulated. Such an embodiment may further be advantageous e.g. for a staggered, e.g. time dependent, administration of the composition of the present invention to a patient in need thereof. The components of such a composition of the present invention as defined herein, particularly the different mRNAs encoding the specific combination of the at least six antigens according to the invention, may be e.g. contained in (different parts of) a kit of parts or may be e.g. administered separately as components of different compositions according to the present invention.

In this context it is particularly preferred that each of the at least six antigens is encoded by a distinct mRNA and is comprised in different parts of a kit. Each mRNA encoding one of the at least six antigens is preferably administered separately as components of different compositions as defined herein. All embodiments disclosed for the inventive composition are applicable for such a combination of compositions comprising mRNAs encoding different antigens.

Preferably, the at least one mRNA of the composition, encoding at least one of the six antigens typically comprises a length of about 50 to about 20000, or 100 to about 20000 nucleotides, preferably of about 250 to about 20000 nucleotides, more preferably of about 500 to about 10000, even more preferably of about 500 to about 5000.

According to one embodiment, the at least one mRNA of the composition, encoding at least one of the six antigens, may be in the form of a modified RNA, wherein any modification, as defined herein, may be introduced into the at least one mRNA of the composition. Modifications as defined herein preferably lead to a stabilized at least one mRNA of the composition of the present invention.

According to a first embodiment, the at least one mRNA of the composition of the present invention may thus be provided as a “stabilized mRNA”, that is to say as an mRNA that is essentially resistant to in vivo degradation (e.g. by an exo- or endo-nuclease). Such stabilization can be effected, for example, by a modified phosphate backbone of the at least one mRNA of the composition of the present invention. A backbone modification in connection with the present invention is a modification in which phosphates of the backbone of the nucleotides contained in the mRNA are chemically modified. Nucleotides that may be preferably used in this connection contain e.g. a phosphorothioate-modified phosphate backbone, preferably at least one of the phosphate oxygens contained in the phosphate backbone being replaced by a sulfur atom. Stabilized mRNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5′-O-(1-thiophosphate)).

The at least one mRNA of the composition of the present invention may additionally or alternatively also contain sugar modifications. A sugar modification in connection with the present invention is a chemical modification of the sugar of the nucleotides of the at least one mRNA and typically includes, without implying any limitation, sugar modifications selected from the group consisting of 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), or azidotriphosphate (2′-azido-2′-deoxycytidine-5′-triphosphate, 2′-azido-2′-deoxyuridine-5′-triphosphate).

The at least one mRNA of the composition of the present invention may additionally or alternatively also contain at least one base modification, which is preferably suitable for increasing the expression of the at least one protein coded for by the at least one mRNA sequence significantly as compared with the unaltered, i.e. natural (=native), mRNA sequence. Significant in this case means an increase in the expression of the protein compared with the expression of the native mRNA sequence by at least 20%, preferably at least 30%, 40%, 50% or 60%, more preferably by at least 70%, 80%, 90% or even 100% and most preferably by at least 150%, 200% or even 300% or more. In connection with the present invention, a nucleotide having such a base modification is preferably selected from the group of the base-modified nucleotides consisting of 2-amino-6-chloropurineriboside-5′-triphosphate, 2-aminoadenosine-5′-triphosphate, 2-thiocytidine-5′-triphosphate, 2-thiouridine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-iodocytidine-5′-triphosphate, 5-iodouridine-5′-triphosphate, 5-methylcytidine-5′-triphosphate, 5-methyluridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

According to another embodiment, the at least one mRNA of the composition of the present invention can likewise be modified (and preferably stabilized) by introducing further modified nucleotides containing modifications of their ribose or base moieties. Generally, the at least one mRNA of the composition of the present invention may contain any native (=naturally occurring) nucleotide, e.g. guanosine, uracil, adenosine, and/or cytosine or an analogue thereof. In this connection, nucleotide analogues are defined as non-natively occurring variants of naturally occurring nucleotides. Accordingly, analogues are chemically derivatized nucleotides with non-natively occurring functional groups, which are preferably added to or deleted from the naturally occurring nucleotide or which substitute the naturally occurring functional groups of a nucleotide. Accordingly, each component of the naturally occurring nucleotide may be modified, namely the base component, the sugar (ribose) component and/or the phosphate component forming the backbone (see above) of the mRNA sequence. Analogues of guanosine, uracil, adenosine, and cytosine include, without implying any limitation, any naturally occurring or non-naturally occurring guanosine, uracil, adenosine, thymidine or cytosine that has been altered chemically, for example by acetylation, methylation, hydroxylation, etc., including 1-methyl-adenosine, 1-methyl-guanosine, 1-methyl-inosine, 2,2-dimethyl-guanosine, 2,6-diaminopurine, 2′-Amino-2′-deoxyadenosine, 2′-Amino-2′-deoxycytidine, 2′-Amino-2′-deoxyguanosine, 2′-Amino-2′-deoxyuridine, 2-Amino-6-chloropurineriboside, 2-Aminopurine-riboside, 2′-Araadenosine, 2′-Aracytidine, 2′-Arauridine, 2′-Azido-2′-deoxyadenosine, 2′-Azido-2′-deoxycytidine, 2′-Azido-2′-deoxyguanosine, 2′-Azido-2′-deoxyuridine, 2-Chloroadenosine, 2′-Fluoro-2′-deoxyadenosine, 2′-Fluoro-2′-deoxycytidine, 2′-Fluoro-2′-deoxyguanosine, 2′-Fluoro-2′-deoxyuridine, 2′-Fluorothymidine, 2-methyl-adenosine, 2-methyl-guanosine, 2-methyl-thio-N6-isopenenyl-adenosine, 2′-O-Methyl-2-aminoadenosine, 2′-O-Methyl-2′-deoxyadenosine, 2′-O-Methyl-2′-deoxycytidine, 2′-O-Methyl-2′-deoxyguanosine, 2′-O-Methyl-2′-deoxyuridine, 2′-O-Methyl-5-methyluridine, 2′-O-Methylinosine, 2′-O-Methylpseudouridine, 2-Thiocytidine, 2-thio-cytosine, 3-methyl-cytosine, 4-acetyl-cytosine, 4-Thiouridine, 5-(carboxyhydroxymethyl)-uracil, 5,6-Dihydrouridine, 5-Aminoallylcytidine, 5-Aminoallyl-deoxy-uridine, 5-Bromouridine, 5-carboxymehtylaminomethyl-2-thio-uracil, 5-carboxymethylamonomethyl-uracil, 5-Chloro-Ara-cytosine, 5-Fluoro-uridine, 5-Iodouridine, 5-methoxycarbonylmethyl-uridine, 5-methoxy-uridine, 5-methyl-2-thio-uridine, 6-Azacytidine, 6-Azauridine, 6-Chloro-7-deaza-guanosine, 6-Chloropurineriboside, 6-Mercapto-guanosine, 6-Methyl-mercaptopurine-riboside, 7-Deaza-2′-deoxy-guanosine, 7-Deazaadenosine, 7-methyl-guanosine, 8-Azaadenosine, 8-Bromo-adenosine, 8-Bromo-guanosine, 8-Mercapto-guanosine, 8-Oxoguanosine, Benzimidazole-riboside, Beta-D-mannosyl-queosine, Dihydro-uracil, Inosine, N1-Methyladenosine, N6-([6-Aminohexyl]carbamoylmethyl)-adenosine, N6-isopentenyl-adenosine, N6-methyl-adenosine, N7-Methyl-xanthosine, N-uracil-5-oxyacetic acid methyl ester, Puromycin, Queosine, Uracil-5-oxyacetic acid, Uracil-5-oxyacetic acid methyl ester, Wybutoxosine, Xanthosine, and Xylo-adenosine. The preparation of such analogues is known to a person skilled in the art, for example from U.S. Pat. No. 4,373,071, U.S. Pat. No. 4,401,796, U.S. Pat. No. 4,415,732, U.S. Pat. No. 4,458,066, U.S. Pat. No. 4,500,707, U.S. Pat. No. 4,668,777, U.S. Pat. No. 4,973,679, U.S. Pat. No. 5,047,524, U.S. Pat. No. 5,132,418, U.S. Pat. No. 5,153,319, U.S. Pat. Nos. 5,262,530 and 5,700,642. In the case of an analogue as described above, particular preference may be given according to the invention to those analogues that increase the immunogenity of the mRNA of the inventive composition and/or do not interfere with a further modification of the mRNA that has been introduced.

According to a particular embodiment, the at least one mRNA of the composition of the present invention can contain a lipid modification. Such a lipid-modified mRNA typically comprises an mRNA as defined herein, encoding at least one of the six antigens as defined above. Such a lipid-modified mRNA typically further comprises at least one linker covalently linked with that mRNA, and at least one lipid covalently linked with the respective linker. Alternatively, the lipid-modified mRNA comprises an (at least one) mRNA as defined herein and at least one (bifunctional) lipid covalently linked (without a linker) with that mRNA. According to a third alternative, the lipid-modified mRNA comprises an mRNA as defined herein, at least one linker covalently linked with that mRNA, and at least one lipid covalently linked with the respective linker, and also at least one (bifunctional) lipid covalently linked (without a linker) with that mRNA.

The lipid contained in the at least one mRNA of the inventive composition (complexed or covalently bound thereto) is typically a lipid or a lipophilic residue that preferably is itself biologically active. Such lipids preferably include natural substances or compounds such as, for example, vitamins, e.g. alpha-tocopherol (vitamin E), including RRR-alpha-tocopherol (formerly D-alpha-tocopherol), L-alpha-tocopherol, the racemate D,L-alpha-tocopherol, vitamin E succinate (VES), or vitamin A and its derivatives, e.g. retinoic acid, retinol, vitamin D and its derivatives, e.g. vitamin D and also the ergosterol precursors thereof, vitamin E and its derivatives, vitamin K and its derivatives, e.g. vitamin K and related quinone or phytol compounds, or steroids, such as bile acids, for example cholic acid, deoxycholic acid, dehydrocholic acid, cortisone, digoxygenin, testosterone, cholesterol or thiocholesterol. Further lipids or lipophilic residues within the scope of the present invention include, without implying any limitation, polyalkylene glycols (Oberhauser et al., Nucl. Acids Res., 1992, 20, 533), aliphatic groups such as, for example, C1-C20-alkanes, C1-C20-alkenes or C1-C20-alkanol compounds, etc., such as, for example, dodecanediol, hexadecanol or undecyl residues (Saison-Behmoaras et al., EMBO J, 1991, 10, 111; Kabanov et al., FEBS Lett., 1990, 259, 327; Svinarchuk et al., Biochimie, 1993, 75, 49), phospholipids such as, for example, phosphatidylglycerol, diacylphosphatidylglycerol, phosphatidylcholine, dipalmitoylphosphatidylcholine, distearoylphosphatidylcholine, phosphatidylserine, phosphatidylethanolamine, di-hexadecyl-rac-glycerol, sphingolipids, cerebrosides, gangliosides, or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), polyamines or polyalkylene glycols, such as, for example, polyethylene glycol (PEG) (Manoharan et al., Nucleosides & Nucleotides, 1995, 14, 969), hexaethylene glycol (HEG), palmitin or palmityl residues (Mishra et al., Biochim. Biophys. Acta, 1995, 1264, 229), octadecylamines or hexylamino-carbonyl-oxycholesterol residues (Crooke et al., J. Pharmacol. Exp. Ther., 1996, 277, 923), and also waxes, terpenes, alicyclic hydrocarbons, saturated and mono- or poly-unsaturated fatty acid residues, etc.

The at least one mRNA of the composition of the present invention may likewise be stabilized in order to prevent degradation of the mRNA in vivo by various approaches. It is known in the art that instability and (fast) degradation of mRNA or of RNA in vivo in general may represent a serious problem in the application of RNA based compositions. This instability of RNA is typically due to RNA-degrading enzymes, “RNases” (ribonucleases), wherein contamination with such ribonucleases may sometimes completely degrade RNA in solution. Accordingly, the natural degradation of mRNA in the cytoplasm of cells is very finely regulated and RNase contaminations may be generally removed by special treatment prior to use of said compositions, in particular with diethyl pyrocarbonate (DEPC). A number of mechanisms of natural degradation are known in this connection in the prior art, which may be utilized as well. E.g., the terminal structure is typically of critical importance for an mRNA in vivo. As an example, at the 5′ end of naturally occurring mRNAs there is usually a so-called “cap structure” (a modified guanosine nucleotide), and at the 3′ end is typically a sequence of up to 200 adenosine nucleotides (the so-called poly-A tail).

The at least one mRNA of the composition of the present invention can therefore be stabilized against degradation by RNases by the addition of a so-called “5′ cap” structure. Particular preference is given in this connection to an m7G(5′)ppp (5′(A,G(5′)ppp(5′)A or G(5′)ppp(5′)G as the 5′ cap” structure. However, such a modification is introduced only if a modification, for example a lipid modification, has not already been introduced at the 5′ end of the mRNA of the inventive composition or if the modification does not interfere with the immunogenic properties of the (unmodified or chemically modified) mRNA.

According to a further preferred embodiment, the at least one mRNA of the composition of the present invention may contain a poly-A tail on the 3′ terminus of typically about 10 to 200 adenosine nucleotides, preferably about 10 to 100 adenosine nucleotides, more preferably about 40 to 80 adenosine nucleotides or even more preferably about 50 to 70 adenosine nucleotides.

According to a further preferred embodiment, the at least one mRNA of the composition of the present invention may contain a poly-C tail on the 3′ terminus of typically about 10 to 200 cytosine nucleotides, preferably about 10 to 100 cytosine nucleotides, more preferably about 20 to 70 cytosine nucleotides or even more preferably about 20 to 60 or even 10 to 40 cytosine nucleotides.

The at least one mRNA according to the invention preferably comprises or codes for at least one histone stem-loop. In the context of the present invention, such a histone stem-loop, in general (irrespective of whether it is a histone stem loop or not), is typically derived from histone genes and comprises an intramolecular base pairing of two neighboring entirely or partially reverse complementary sequences, thereby forming a stem-loop. A stem-loop can occur in single-stranded DNA or, more commonly, in RNA.

In the context of the present application, a histone stem-loop sequence may be described by its DNA or by its corresponding RNA sequence. Thus, any reference—throughout the present application—to histone stem-loop sequences, which are represented herein by DNA sequences (e.g. SEQ ID NO: 37 to 66 and 70), also comprises the corresponding RNA sequence. This applies in particular to histone stem-loop sequences, which are comprised in the at least one mRNA according to the invention. Accordingly, by reference to a specific DNA sequence that defines a histone stem-loop, the corresponding RNA sequence is defined as well.

The structure is also known as a hairpin or hairpin loop and usually consists of a stem and a (terminal) loop within a consecutive sequence, wherein the stem is formed by two neighbored entirely or partially reverse complementary sequences separated by a short sequence as sort of spacer, which builds the loop of the stem-loop structure. The two neighbored entirely or partially reverse complementary sequences may be defined as e.g. stem loop elements stem1 and stem2. The stem loop is formed when these two neighbored entirely or partially reverse complementary sequences, e.g. stem loop elements stem1 and stem2, form base-pairs with each other, leading to a double stranded nucleic acid sequence stretch comprising an unpaired loop at its terminal ending formed by the short sequence located between stem loop elements stem1 and stem2 on the consecutive sequence. The unpaired loop thereby typically represents a region of the nucleic acid which is not capable of base pairing with either of these stem loop elements. The resulting lollipop-shaped structure is a key building block of many RNA secondary structures. The formation of a stem-loop structure is thus dependent on the stability of the resulting stem and loop regions, wherein the first prerequisite is typically the presence of a sequence that can fold back on itself to form a paired double strand. The stability of paired stem loop elements is determined by the length, the number of mismatches or bulges it contains (a small number of mismatches is typically tolerable, especially in a long double stranded stretch), and the base composition of the paired region. In the context of the present invention, a loop length of 3 to 15 bases is conceivable, while a more preferred loop length is 3-10 bases, more preferably 3 to 8, 3 to 7, 3 to 6 or even more preferably 4 to 5 bases, and most preferably 4 bases. The sequence forming the stem region in the histone stem-loop typically has a length of between 5 to 10 bases, more preferably, between 5 to 8 bases, wherein preferably at least one of the bases represents a mismatch, i.e. does not base pair.

In the context of the present invention, a histone stem-loop is typically derived from histone genes (e.g. genes from the histone families H1, H2A, H2B, H3, H4) and comprises an intramolecular base pairing of two neighbored entirely or partially reverse complementary sequences, thereby forming a stem-loop. Typically, a histone 3′ UTR stem-loop is an RNA element involved in nucleocytoplasmic transport of the histone mRNAs, and in the regulation of stability and of translation efficiency in the cytoplasm. The mRNAs of metazoan histone genes lack polyadenylation and a poly-A tail, instead 3′ end processing occurs at a site between this highly conserved stem-loop and a purine rich region around 20 nucleotides downstream (the histone downstream element, or HDE). The histone stem-loop is bound by a 31 kDa stem-loop binding protein (SLBP—also termed the histone hairpin binding protein, or HBP). Such histone stem-loop structures are preferably employed by the present invention in combination with other sequence elements and structures, which do not occur naturally (which means in untransformed living organisms/cells) in histone genes, but are combined—according to the invention—to provide an artificial, heterologous nucleic acid. Accordingly, it was found that an artificial (non-native) combination of a histone stem-loop structure with other heterologous sequence elements, which do not occur in histone genes or metazoan histone genes and are isolated from operational and/or regulatory sequence regions (influencing transcription and/or translation) of genes coding for proteins other than histones, provide advantageous effects. Accordingly, one embodiment of the invention comprises the combination of a histone stem-loop structure with a poly(A) sequence or a sequence representing a polyadenylation signal (3′-terminal of a coding region), which does not occur in metazoan histone genes. According to another preferred embodiment of the invention, a combination of a histone stem-loop structure with a coding region coding for at least one of the antigens according to the invention as defined above, which does, preferably not occur in metazoan histone genes, is provided herewith (coding region and histone stem loop sequence are heterologous).

A histone stem loop is, therefore, a stem-loop structure as described herein, which, if preferably functionally defined, exhibits/retains the property of binding to its natural binding partner, the stem-loop binding protein (SLBP—also termed the histone hairpin binding protein, or HBP).

In a preferred embodiment, the histone stem loop sequence is not derived from a mouse histone protein. More specifically, the histone stem loop sequence may not be derived from mouse histone gene H2A614. Also, the at least one mRNA according to the invention may neither contain a mouse histone stem loop sequence nor contain mouse histone gene H2A614. Further, the at least one mRNA according to the invention may not contain a stem-loop processing signal, more specifically, a mouse histone processing signal and, most specifically, may not contain mouse stem loop processing signal H2kA614, even if the at least one mRNA contains at least one mammalian histone gene. However, the at least one mammalian histone gene may not be Seq. ID No. 7 of WO 01/12824.

The at least one mRNA as define above preferably comprises a coding region encoding the antigens as defined above or a fragment, variant or derivative thereof; and a 3′ UTR containing at least one histone stem-loop. When in addition to the antigens defined above, a further peptide or protein is encoded by the at least one mRNA, then the encoded peptide or protein is preferably no histone protein, no reporter protein and/or no marker or selection protein, as defined above. The 3′ UTR of the at least one mRNA preferably comprises also a poly(A) and/or a poly(C) sequence as defined herewithin. The single elements of the 3′ UTR may occur therein in any order from 5′ to 3′ along the sequence of the at least one mRNA. In addition, further elements as described herein, may also be contained, such as a stabilizing sequence as defined herewithin (e.g. derived from the UTR of a globin gene), IRES sequences, etc. Each of the elements may also be repeated in the at least one mRNA according to the invention at least once (particularly in di- or multicistronic constructs), preferably twice or more. As an example, the single elements may be present in the at least one mRNA in the following order:

5′-coding region-histone stem-loop-poly(A)/(C) sequence-3′; or
5′-coding region-poly(A)/(C) sequence-histone stem-loop-3′; or
5′-coding region-histone stem-loop-polyadenylation signal-3′; or
5′-coding region-polyadenylation signal-histone stem-loop-3′; or
5′-coding region-histone stem-loop-histone stem-loop-poly(A)/(C) sequence-3′; or
5′-coding region-histone stem-loop-histone stem-loop-polyadenylation signal-3′; or
5′-coding region-stabilizing sequence-poly(A)/(C) sequence-histone stem-loop-3′; or
5′-coding region-stabilizing sequence-poly(A)/(C) sequence-poly(A)/(C) sequence-histone stem-loop-3′; etc.

In this context, it is particularly preferred that—if, in addition to the antigens defined above, a further peptide or protein is encoded by the at least one mRNA—the encoded peptide or protein is preferably no histone protein, no reporter protein (e.g. Luciferase, GFP, EGFP, β-Galactosidase, particularly EGFP) and/or no marker or selection protein (e.g. alpha-Globin, Galactokinase and Xanthine:Guanine phosphoribosyl transferase (GPT)).

In a preferred embodiment, the mRNA according to the invention does not comprise a reporter gene or a marker gene. Preferably, the mRNA according to the invention does not encode, for instance, luciferase; green fluorescent protein (GFP) and its variants (such as eGFP, RFP or BFP); α-globin; hypoxanthine-guanine phosphoribosyltransferase (HGPRT); β-galactosidase; galactokinase; alkaline phosphatase; secreted embryonic alkaline phosphatase (SEAP)) or a resistance gene (such as a resistance gene against neomycin, puromycin, hygromycin and zeocin). In a preferred embodiment, the mRNA according to the invention does not encode luciferase. In another embodiment, the mRNA according to the invention does not encode GFP or a variant thereof.

In a further preferred embodiment, the mRNA according to the invention does not encode a protein (or a fragment of a protein) derived from a virus, preferably from a virus belonging to the family of Orthomyxoviridae. Preferably the mRNA does not encode a protein that is derived from an influenza virus, more preferably an influenza A virus. Preferably, the mRNA according to the invention does not encode an influenza A protein selected from the group consisting of hemagglutinin (HA), neuraminidase (NA), nucleoprotein (NP), M1, M2, NS1, NS2 (NEP: nuclear export protein), PA, PB1 (polymerase basic 1), PB1-F2 and PB2. In another preferred embodiment, the mRNA according to the invention does not encode ovalbumin (OVA) or a fragment thereof. Preferably, the mRNA according to the invention does not encode an influenza A protein or ovalbumin.

According to one preferred embodiment, the at least one mRNA according to the invention comprises at least one histone stem-loop sequence, preferably according to at least one of the following formulae (I) or (II):

formula (I) (stem-loop sequence without stem bordering elements):

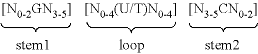

formula (II) (stem-loop sequence with stem bordering elements):

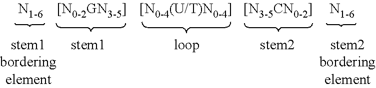

wherein:
stem1 or stem2 bordering elements N₁₋₆ is a consecutive sequence of 1 to 6, preferably of 2 to 6, more preferably of 2 to 5, even more preferably of 3 to 5, most preferably of 4 to 5 or 5 N, wherein each N is independently from another selected from a nucleotide selected from A, U, T, G and C, or a nucleotide analogue thereof;
stem1 [N₀₋₂GN₃₋₅] is reverse complementary or partially reverse complementary with element stem2, and is a consecutive sequence between of 5 to 7 nucleotides;
   wherein N₀₋₂ is a consecutive sequence of 0 to 2, preferably of 0 to 1, more preferably of 1 N, wherein each N is independently from another selected from a nucleotide selected from A, U, T, G and C or a nucleotide analogue thereof;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof, and
   wherein G is guanosine or an analogue thereof, and may be optionally replaced by a cytidine or an analogue thereof, provided that its complementary nucleotide cytidine in stem2 is replaced by guanosine;
loop sequence [N₀₋₄(U/T)N₀₋₄] is located between elements stem1 and stem2, and is a consecutive sequence of 3 to 5 nucleotides, more preferably of 4 nucleotides;
   wherein each N₀₋₄ is independent from another a consecutive sequence of 0 to 4, preferably of 1 to 3, more preferably of 1 to 2 N, wherein each N is independently from another selected from a nucleotide selected from A, U, T, G and C or a nucleotide analogue thereof; and
   wherein U/T represents uridine, or optionally thymidine;
stem2 [N₃₋₅CN₀₋₂] is reverse complementary or partially reverse complementary with element stem1, and is a consecutive sequence between of 5 to 7 nucleotides;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof;
   wherein N₀₋₂ is a consecutive sequence of 0 to 2, preferably of 0 to 1, more preferably of 1 N, wherein each N is independently from another selected from a nucleotide selected from A, U, T, G or C or a nucleotide analogue thereof; and
   wherein C is cytidine or an analogue thereof, and may be optionally replaced by a guanosine or an analogue thereof provided that its complementary nucleotide guanosine in stem1 is replaced by cytidine;
   wherein
   stem1 and stem2 are capable of base pairing with each other forming a reverse complementary sequence, wherein base pairing may occur between stem1 and stem2, e.g. by Watson-Crick base pairing of nucleotides A and U/T or G and C or by non-Watson-Crick base pairing e.g. wobble base pairing, reverse Watson-Crick base pairing, Hoogsteen base pairing, reverse Hoogsteen base pairing or are capable of base pairing with each other forming a partially reverse complementary sequence, wherein an incomplete base pairing may occur between stem1 and stem2, on the basis that one or more bases in one stem do not have a complementary base in the reverse complementary sequence of the other stem.

In the above context, a wobble base pairing is typically a non-Watson-Crick base pairing between two nucleotides. The four main wobble base pairs in the present context, which may be used, are guanosine-uridine, inosine-uridine, inosine-adenosine, inosine-cytidine (G-U/T, I-U/T, I-A and I-C) and adenosine-cytidine (A-C).

Accordingly, in the context of the present invention, a wobble base is a base, which forms a wobble base pair with a further base as described above. Therefore non-Watson-Crick base pairing, e.g. wobble base pairing, may occur in the stem of the histone stem-loop structure in the at least one mRNA according to the present invention.

In the above context, a partially reverse complementary sequence comprises maximally 2, preferably only one mismatch in the stem-structure of the stem-loop sequence formed by base pairing of stem1 and stem2. In other words, stem1 and stem2 are preferably capable of (full) base pairing with each other throughout the entire sequence of stem1 and stem2 (100% of possible correct Watson-Crick or non-Watson-Crick base pairings), thereby forming a reverse complementary sequence, wherein each base has its correct Watson-Crick or non-Watson-Crick base pendant as a complementary binding partner. Alternatively, stem1 and stem2 are preferably capable of partial base pairing with each other throughout the entire sequence of stem1 and stem2, wherein at least about 70%, 75%, 80%, 85%, 90%, or 95% of the 100% possible correct Watson-Crick or non-Watson-Crick base pairings are occupied with the correct Watson-Crick or non-Watson-Crick base pairings and at most about 30%, 25%, 20%, 15%, 10%, or 5% of the remaining bases are unpaired.

According to a preferred embodiment, the at least one histone stem-loop sequence (with stem bordering elements) of the at least one mRNA as defined herein comprises a length of about 15 to about 45 nucleotides, preferably a length of about 15 to about 40 nucleotides, preferably a length of about 15 to about 35 nucleotides, preferably a length of about 15 to about 30 nucleotides and even more preferably a length of about 20 to about 30 and most preferably a length of about 24 to about 28 nucleotides.

According to a further preferred embodiment, the at least one histone stem-loop sequence (without stem bordering elements) of the at least one mRNA as defined herein comprises a length of about 10 to about 30 nucleotides, preferably a length of about 10 to about 20 nucleotides, preferably a length of about 12 to about 20 nucleotides, preferably a length of about 14 to about 20 nucleotides and even more preferably a length of about 16 to about 17 and most preferably a length of about 16 nucleotides.

According to a further preferred embodiment, the at least one mRNA according to the present invention may comprise at least one histone stem-loop sequence according to at least one of the following specific formulae (Ia) or (IIa):

formula (Ia) (stem-loop sequence without stem bordering elements):

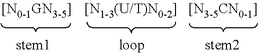

formula (IIa) (stem-loop sequence with stem bordering elements):

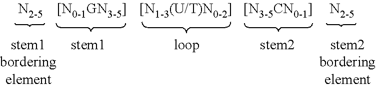

wherein:
N, C, G, T and U are as defined above.

According to a further more particularly preferred embodiment of the first aspect, the at least one mRNA may comprise or code for at least one histone stem-loop sequence according to at least one of the following specific formulae (Ib) or (IIb):

formula (Ib) (stem-loop sequence without stem bordering elements):

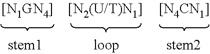

formula (IIb) (stem-loop sequence with stem bordering elements):

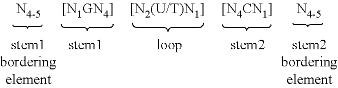

wherein:
N, C, G, T and U are as defined above.

According to an even more preferred embodiment, the at least one mRNA according to the present invention may comprise at least one histone stem-loop sequence according to at least one of the following specific formulae (Ic) to (Ih) or (IIc) to (IIh), shown alternatively in its stem-loop structure and as a linear sequence representing histone stem-loop sequences:

formula (Ic): (metazoan and protozoan histone stem-loop consensus sequence without stem bordering elements):

```
  N U
N N
  N-N
  N-N
  N-N
  N-N
  G-C
  N-N (stem-loop structure)
(SEQ ID NO: 25)
NGNNNNNNUNNNNNCN
(linear sequence)
```

formula (IIc): (metazoan and protozoan histone stem-loop consensus sequence with stem bordering elements):

```
            N U
          N N
            N-N
            N-N
            N-N
            N-N
            G-C
N*N*NNNN-NNNN*N*N* (stem-loop structure)
(SEQ ID NO: 26)
N*N*NNNNGNNNNNNUNNNNNCNNNN*N*N*
(linear sequence)
```

formula (Id): (without stem bordering elements)

```
  N U
N N
  N-N
  N-N
  N-N
  N-N
  C-G
  N-N (stem-loop structure)
(SEQ ID NO: 27)
NCNNNNNNUNNNNNGN
(linear sequence)
```

formula (IId): (with stem bordering elements)

```
  N U
N N
  N-N
  N-N
  N-N
  N-N
  C-G
N*N*NNNN-NNNN*N*N* (stem-loop structure)
(SEQ ID NO: 28)
N*N*NNNNCNNNNNNUNNNNNGNNNN*N*N*
(linear sequence)
```

formula (Ie): (protozoan histone stem-loop consensus sequence without stem bordering elements)

```
  N U
N N
  N-N
  N-N
  N-N
  N-N
  G-C
  D-H (stem-loop structure)
(SEQ ID NO: 29)
DGNNNNNNUNNNNNCH
(linear sequence)
```

formula (IIe): (protozoan histone stem-loop consensus sequence with stem bordering elements)

```
  N U
N N
  N-N
  N-N
  N-N
  N-N
  G-C
N*N*NNND-HNNN*N*N* (stem-loop structure)
(SEQ ID NO: 30)
N*N*NNNDGNNNNNNUNNNNNCHNNN*N*N*
(linear sequence)
```

formula (If): (metazoan histone stem-loop consensus sequence without stem bordering elements)

```
  N U
N N
  Y-V
  Y-N
  B-D
  N-N
  G-C
  N-N (stem-loop structure)
(SEQ ID NO: 31)
NGNBYYNNUNVNDNCN
(linear sequence)
```

formula (IIf): (metazoan histone stem-loop consensus sequence with stem bordering elements)

```
  N U
N N
  Y-V
  Y-N
  B-D
  N-N
  G-C
N*N*NNNN-NNNN*N*N* (stem-loop structure)
(SEQ ID NO: 32)
N*N*NNNNGNBYYNNUNVNDNCNNNN*N*N*
(linear sequence)
```

formula (Ig): (vertebrate histone stem-loop consensus sequence without stem bordering elements)

```
  N U
D H
  Y-A
  Y-B
  Y-R
  H-D
  G-C
  N-N (stem-loop structure)
(SEQ ID NO: 33)
NGHYYYDNUHABRDCN
(linear sequence)
```

formula (IIg): (vertebrate histone stem-loop consensus sequence with stem bordering elements)

```
  N U
D H
  Y-A
  Y-B
  Y-R
  H-D
  G-C
N*N*HNNN-NNNN*N*H* (stem-loop structure)
(SEQ ID NO: 34)
N*N*HNNNGHYYYDNUHABRDCNNNN*N*H*
(linear sequence)
```

formula (Ih): (human histone stem-loop consensus sequence (Homo sapiens) without stem bordering elements)

```
  Y U
D H
  U-A
  C-S
  Y-R
  H-R
  G-C
  D-C (stem-loop structure)
(SEQ ID NO: 35)
DGHYCUDYUHASRRCC
(linear sequence)
```

formula (IIh): (human histone stem-loop consensus sequence (Homo sapiens) with stem bordering elements)

```
  Y U
D H
  U-A
  C-S
  Y-R
  H-R
  G-C
N*H*AAHD-CVHB*N*H* (stem loop structure)
(SEQ ID NO: 36)
N*H*AAHDGHYCUDYUHASRRCCVHB*N*H*
(linear sequence)
```

wherein in each of above formulae (Ic) to (Ih) or (IIc) to (IIh):
N, C, G, A, T and U are as defined above;
each U may be replaced by T;
each (highly) conserved G or C in the stem elements 1 and 2 may be replaced by its complementary nucleotide base C or G, provided that its complementary nucleotide in the corresponding stem is replaced by its complementary nucleotide in parallel; and/or
G, A, T, U, C, R, Y, M, K, S, W, H, B, V, D, and N are nucleotide bases as defined in the following Table:

| abbreviation | Nucleotide bases | remark |
|---|---|---|
| G | G | Guanine |
| A | A | Adenine |
| T | T | Thymine |
| U | U | Uracile |
| C | C | Cytosine |
| R | G or A | Purine |
| Y | T/U or C | Pyrimidine |
| M | A or C | Amino |
| K | G or T/U | Keto |
| S | G or C | Strong (3H bonds) |
| W | A or T/U | Weak (2H bonds) |
| H | A or C or T/U | Not G |
| B | G or T/U or C | Not A |
| V | G or C or A | Not T/U |
| D | G or A or T/U | Not C |
| N | G or C or T/U or A | Any base |
| * | Present or not | Base may be present or not |

In this context it is particularly preferred that the histone stem-loop sequence according to at least one of the formulae (I) or (Ia) to (Ih) or (II) or (IIa) to (IIh) above is selected from a naturally occurring histone stem loop sequence, more particularly preferred from protozoan or metazoan histone stem-loop sequences, and even more particularly preferred from vertebrate and mostly preferred from mammalian histone stem-loop sequences especially from human histone stem-loop sequences.

According to a particularly preferred embodiment the histone stem-loop sequence according to at least one of the specific formulae (I) or (Ia) to (Ih) or (II) or (IIa) to (IIh) is a histone stem-loop sequence comprising at each nucleotide position the most frequently occurring nucleotide, or either the most frequently or the second-most frequently occurring nucleotide of naturally occurring histone stem-loop sequences in metazoa and protozoa, protozoa, metazoa, vertebrates and humans. In this context it is particularly preferred that at least 80%, preferably at least 85%, or most preferably at least 90% of all nucleotides correspond to the most frequently occurring nucleotide of naturally occurring histone stem-loop sequences.

In a further particular embodiment, the histone stem-loop sequence according to at least one of the specific formulae (I) or (Ia) to (Ih) above is selected from following histone stem-loop sequences (without stem-bordering elements):

(SEQ ID NO: 37 according to formula (Ic))
VGYYYYHHTHRVVRCB
(SEQ ID NO: 38 according to formula (Ic))
SGYYYTTYTMARRRCS
(SEQ ID NO: 39 according to formula (Ic))
SGYYCTTTTMAGRRCS
(SEQ ID NO: 40 according to formula (Ie))
DGNNNBNNTHVNNNCH
(SEQ ID NO: 41 according to formula (Ie))
RGNNNYHBTHRDNNCY
(SEQ ID NO: 42 according to formula (Ie))
RGNDBYHYTHRDHNCY
(SEQ ID NO: 43 according to formula (If))
VGYYYTYHTHRVRRCB
(SEQ ID NO: 44 according to formula (If))
SGYYCTTYTMAGRRCS
(SEQ ID NO: 45 according to formula (If))
SGYYCTTTTMAGRRCS
(SEQ ID NO: 46 according to formula (Ig))
GGYYCTTYTHAGRRCC
(SEQ ID NO: 47 according to formula (Ig))
GGCYCTTYTMAGRGCC
(SEQ ID NO: 48 according to formula (Ig))
GGCTCTTTTMAGRGCC
(SEQ ID NO: 49 according to formula (Ih))
DGHYCTDYTHASRRCC
(SEQ ID NO: 50 according to formula (Ih))
GGCYCTTTTHAGRGCC
(SEQ ID NO: 51 according to formula (Ih))
GGCYCTTTTMAGRGCC

Furthermore in this context following histone stem-loop sequences (with stem bordering elements) according to one of specific formulae (II) or (IIa) to (IIh) are particularly preferred:

(SEQ ID NO: 52 according to formula (IIc))
H*H*HHVVGYYYYHHTHRVVRCBVHH*N*N*
(SEQ ID NO: 53 according to formula (IIc))
M*H*MHMSGYYYTTYTMARRRCSMCH*H*H*
(SEQ ID NO: 54 according to formula (IIc))
M*M*MMMSGYYCTTTTMAGRRCSACH*M*H*
(SEQ ID NO: 55 according to formula (IIe))
N*N*NNNDGNNNBNNTHVNNNCHNHN*N*N*
(SEQ ID NO: 56 according to formula (IIe))
N*N*HHNRGNNNYHBTHRDNNCYDHH*N*N*
(SEQ ID NO: 57 according to formula (IIe))
N*H*HHVRGNDBYHYTHRDHNCYRHH*H*H*
(SEQ ID NO: 58 according to formula (IIf))
H*H*MHMVGYYYTYHTHRVRRCBVMH*H*N*
(SEQ ID NO: 59 according to formula (IIf))
M*M*MMMSGYYCTTYTMAGRRCSMCH*H*H*
(SEQ ID NO: 60 according to formula (IIf))
M*M*MMMSGYYCTTTTMAGRRCSACH*M*H*
(SEQ ID NO: 61 according to formula (IIg))
H*H*MAMGGYYCTTYTHAGRRCCVHN*N*M*
(SEQ ID NO: 62 according to formula (IIg))
H*H*AAMGGCYCTTYTMAGRGCCVCH*H*M*
(SEQ ID NO: 63 according to formula (IIg))
M*M*AAMGGCTCTTTTMAGRGCCMCY*M*M*
(SEQ ID NO: 64 according to formula (IIh))
N*H*AAHDGHYCTDYTHASRRCCVHB*N*H*
(SEQ ID NO: 65 according to formula (IIh))
H*H*AAMGGCYCTTTTHAGRGCCVMY*N*M*
(SEQ ID NO: 66 according to formula (IIh))
H*M*AAAGGCYCTTTTMAGRGCCRMY*H*M*

According to a further preferred embodiment the at least one mRNA of the composition according to the present invention comprises at least one histone stem-loop sequence showing at least about 80%, preferably at least about 85%, more preferably at least about 90%, or even more preferably at least about 95%, sequence identity with the not to 100% conserved nucleotides in the histone stem-loop sequences according to at least one of specific formulae (I) or (Ia) to (Ih) or (II) or (IIa) to (IIh) or with a naturally occurring histone stem-loop sequence.

A particular preferred histone stem-loop sequence is the sequence according to SEQ ID NO: 70 CAAAGGCTCTTTTCAGAGCCACCA or more preferably the corresponding RNA sequence of the nucleic acid sequence according to SEQ ID NO: 70:

| | |
|---|---|
| CAAAGGCUCUUUUCAGAGCCACCA. | (SEQ ID NO: 71) |

In a preferred embodiment, the histone stem loop sequence does not contain the loop sequence 5′-UUUC-3′. More specifically, the histone stem loop does not contain the stem1 sequence 5′-GGCUCU-3′ and/or the stem2 sequence 5′-AGAGCC-3′, respectively. In another preferred embodiment, the stem loop sequence does not contain the loop sequence 5′-CCUGCCC-3′ or the loop sequence 5′-UGAAU-3′. More specifically, the stem loop does not contain the stem1 sequence 5′-CCUGAGC-3′ or does not contain the stem1 sequence 5′-ACCUUUCUCCA-3′ and/or the stem2 sequence 5′-GCUCAGG-3′ or 5′-UGGAGAAAGGU-3′, respectively. Also, stem loop sequences are preferably not derived from a mammalian insulin receptor 3′-untranslated region. Also, preferably, the at least one mRNA according to the invention may not contain histone stem loop processing signals, in particular not those derived from mouse histone gene H2A614 gene (H2kA614).

Preferably, the at least one mRNA of the composition according to the present invention does not contain one or two or at least one or all but one or all of the components of the group consisting of: a sequence encoding a ribozyme (preferably a self-splicing ribozyme), a viral nucleic acid sequence, a histone stem-loop processing signal, in particular a histone-stem loop processing sequence derived from mouse histone H2A614 gene, a Neo gene, an inactivated promoter sequence and an inactivated enhancer sequence. Even more preferably, the at least one mRNA according to the invention does not contain a ribozyme, preferably a self-splicing ribozyme, and one of the group consisting of: a Neo gene, an inactivated promoter sequence, an inactivated enhancer sequence, a histone stem-loop processing signal, in particular a histone-stem loop processing sequence derived from mouse histone H2A614 gene. Accordingly, the mRNA may in a preferred mode neither contain a ribozyme, preferably a self-splicing ribozyme, nor a Neo gene or, alternatively, neither a ribozyme, preferably a self-splicing ribozyme, nor any resistance gene (e.g. usually applied for selection). In another preferred mode, the at least one mRNA of the invention may neither contain a ribozyme, preferably a self-splicing ribozyme nor a histone stem-loop processing signal, in particular a histone-stem loop processing sequence derived from mouse histone H2A614 gene

Alternatively, the at least one mRNA of the composition according to the invention optionally comprises a polyadenylation signal which is defined herein as a signal which conveys polyadenylation to a (transcribed) mRNA by specific protein factors (e.g. cleavage and polyadenylation specificity factor (CPSF), cleavage stimulation factor (CstF), cleavage factors I and II (CF I and CF II), poly(A) polymerase (PAP)). In this context a consensus polyadenylation signal is preferred comprising the NN(U/T)ANA consensus sequence. In a particular preferred aspect the polyadenylation signal comprises one of the following sequences: AA(U/T)AAA or A(U/T)(U/T)AAA (wherein uridine is usually present in RNA and thymidine is usually present in DNA). In some embodiments, the polyadenylation signal used in the at least one mRNA according to the invention does not correspond to the U3 snRNA, U5, the polyadenylation processing signal from human gene G-CSF, or the SV40 polyadenylation signal sequences. In particular, the above polyadenylation signals are not combined with any antibiotics resistance gene (or any other reporter, marker or selection gene), in particular not with the resistance neo gene (neomycin phosphotransferase). And any of the above polyadenylation signals are preferably not combined with the histone stem loop or the histone stem loop processing signal from mouse histone gene H2A614 in the at least one mRNA according to the invention.

According to another embodiment, the at least one mRNA of the composition of the present invention may be modified, and thus stabilized by modifying the G/C content of the mRNA, preferably of the coding region of the at least one mRNA.

In a particularly preferred embodiment of the present invention, the G/C content of the coding region of the at least one mRNA of the composition of the present invention is modified, particularly increased, compared to the G/C content of the coding region of its particular wild-type mRNA, i.e. the unmodified mRNA. The amino acid sequence encoded by the at least one mRNA is preferably not modified as compared to the amino acid sequence encoded by the particular wild-type mRNA. This modification of the at least one mRNA of the composition of the present invention is based on the fact that the sequence of any mRNA region to be translated is important for efficient translation of that mRNA. Thus, the composition and the sequence of various nucleotides are important. In particular, sequences having an increased G (guanosine)/C (cytosine) content are more stable than sequences having an increased A (adenosine)/U (uracil) content. According to the invention, the codons of the mRNA are therefore varied compared to the respective wild-type mRNA, while retaining the translated amino acid sequence, such that they include an increased amount of G/C nucleotides. In respect to the fact that several codons code for one and the same amino acid (so-called degeneration of the genetic code), the most favorable codons for the stability can be determined (so-called alternative codon usage). Depending on the amino acid to be encoded by the at least one mRNA, there are various possibilities for modification of the mRNA sequence, compared to its wild-type sequence. In the case of amino acids which are encoded by codons which contain exclusively G or C nucleotides, no modification of the codon is necessary. Thus, the codons for Pro (CCC or CCG), Arg (CGC or CGG), Ala (GCC or GCG) and Gly (GGC or GGG) require no modification, since no A or U is present. In contrast, codons which contain A and/or U nucleotides can be modified by substitution of other codons which code for the same amino acids but contain no A and/or U. Examples of these are: the codons for Pro can be modified from CCU or CCA to CCC or CCG; the codons for Arg can be modified from CGU or CGA or AGA or AGG to CGC or CGG; the codons for Ala can be modified from GCU or GCA to GCC or GCG; the codons for Gly can be modified from GGU or GGA to GGC or GGG. In other cases, although A or U nucleotides cannot be eliminated from the codons, it is however possible to decrease the A and U content by using codons which contain a lower content of A and/or U nucleotides. Examples of these are: the codons for Phe can be modified from UUU to UUC; the codons for Leu can be modified from UUA, UUG, CUU or CUA to CUC or CUG; the codons for Ser can be modified from UCU or UCA or AGU to UCC, UCG or AGC; the codon for Tyr can be modified from UAU to UAC; the codon for Cys can be modified from UGU to UGC; the codon for His can be modified from CAU to CAC; the codon for Gln can be modified from CAA to CAG; the codons for Ile can be modified from AUU or AUA to AUC; the codons for Thr can be modified from ACU or ACA to ACC or ACG; the codon for Asn can be modified from AAU to AAC; the codon for Lys can be modified from AAA to AAG; the codons for Val can be modified from GUU or GUA to GUC or GUG; the codon for Asp can be modified from GAU to GAC; the codon for Glu can be modified from GAA to GAG; the stop codon UAA can be modified to UAG or UGA. In the case of the codons for Met (AUG) and Trp (UGG), on the other hand, there is no possibility of sequence modification. The substitutions listed above can be used either individually or in all possible combinations to increase the G/C content of the at least one mRNA of the composition of the present invention compared to its particular wild-type mRNA (i.e. the original sequence). Thus, for example, all codons for Thr occurring in the wild-type sequence can be modified to ACC (or ACG). Preferably, however, for example, combinations of the above substitution possibilities are used:

substitution of all codons coding for Thr in the original sequence (wild-type mRNA) to ACC (or ACG) and
substitution of all codons originally coding for Ser to UCC (or UCG or AGC); substitution of all codons coding for Ile in the original sequence to AUC and
substitution of all codons originally coding for Lys to AAG and
substitution of all codons originally coding for Tyr to UAC; substitution of all codons coding for Val in the original sequence to GUC (or GUG) and
substitution of all codons originally coding for Glu to GAG and
substitution of all codons originally coding for Ala to GCC (or GCG) and
substitution of all codons originally coding for Arg to CGC (or CGG); substitution of all codons coding for Val in the original sequence to GUC (or GUG) and
substitution of all codons originally coding for Glu to GAG and
substitution of all codons originally coding for Ala to GCC (or GCG) and
substitution of all codons originally coding for Gly to GGC (or GGG) and
substitution of all codons originally coding for Asn to AAC; substitution of all codons coding for Val in the original sequence to GUC (or GUG) and
substitution of all codons originally coding for Phe to UUC and
substitution of all codons originally coding for Cys to UGC and
substitution of all codons originally coding for Leu to CUG (or CUC) and
substitution of all codons originally coding for Gln to CAG and
substitution of all codons originally coding for Pro to CCC (or CCG); etc. Preferably, the G/C content of the coding region of the at least one mRNA of the composition of the present invention is increased by at least 7%, more preferably by at least 15%, particularly preferably by at least 20%, compared to the G/C content of the coded region of the wild-type mRNA which codes for an antigen, antigenic protein or antigenic peptide as defined herein or its fragment or variant thereof. According to a specific embodiment at least 5%, 10%, 20%, 30%, 40%, 50%, 60%, more preferably at least 70%, even more preferably at least 80% and most preferably at least 90%, 95% or even 100% of the substitutable codons in the region coding for an antigen, antigenic protein or antigenic peptide as defined herein or its fragment or variant thereof or the whole sequence of the wild type mRNA sequence are substituted, thereby increasing the GC/content of said sequence. In this context, it is particularly preferable to increase the G/C content of the at least one (m)RNA of the composition of the present invention to the maximum (i.e. 100% of the substitutable codons), in particular in the region coding for a protein, compared to the wild-type sequence. According to the invention, a further preferred modification of the at least one mRNA of the composition of the present invention is based on the finding that the translation efficiency is also determined by a different frequency in the occurrence of tRNAs in cells. Thus, if so-called “rare codons” are present in the at least one mRNA of the composition of the present invention to an increased extent, the corresponding modified at least one mRNA sequence is translated to a significantly poorer degree than in the case where codons coding for relatively “frequent” tRNAs are present. According to the invention, in the modified at least one mRNA of the composition of the present invention, the region which codes for the antigen is modified compared to the corresponding region of the wild-type mRNA such that at least one codon of the wild type sequence which codes for a tRNA which is relatively rare in the cell is exchanged for a codon which codes for a tRNA which is relatively frequent in the cell and carries the same amino acid as the relatively rare tRNA. By this modification, the sequences of the at least one mRNA of the composition of the present invention is modified such that codons for which frequently occurring tRNAs are available are inserted. In other words, according to the invention, by this modification all codons of the wild type sequence which code for a tRNA which is relatively rare in the cell can in each case be exchanged for a codon which codes for a tRNA which is relatively frequent in the cell and which, in each case, carries the same amino acid as the relatively rare tRNA. Which tRNAs occur relatively frequently in the cell and which, in contrast, occur relatively rarely is known to a person skilled in the art; cf. e.g. Akashi, Curr. Opin. Genet. Dev. 2001, 11(6): 660-666. The codons which use for the particular amino acid the tRNA which occurs the most frequently, e.g. the Gly codon, which uses the tRNA which occurs the most frequently in the (human) cell, are particularly preferred. According to the invention, it is particularly preferable to link the sequential G/C content which is increased, in particular maximized, in the modified at least one mRNA of the composition of the present invention, with the “frequent” codons without modifying the amino acid sequence of the protein encoded by the coding region of the mRNA. This preferred embodiment allows provision of a particularly efficiently translated and stabilized (modified) at least one mRNA of the composition of the present invention. The determination of a modified at least one mRNA of the composition of the present invention as described above (increased G/C content; exchange of tRNAs) can be carried out using the computer program explained in WO 02/098443—the disclosure content of which is included in its full scope in the present invention. Using this computer program, the nucleotide sequence of any desired mRNA can be modified with the aid of the genetic code or the degenerative nature thereof such that a maximum G/C content results, in combination with the use of codons which code for tRNAs occurring as frequently as possible in the cell, the amino acid sequence coded by the modified at least one mRNA preferably not being modified compared to the non-modified sequence. Alternatively, it is also possible to modify only the G/C content or only the codon usage compared to the original sequence. The source code in Visual Basic 6.0 (development environment used: Microsoft Visual Studio Enterprise 6.0 with Servicepack 3) is also described in WO 02/098443. In a further preferred embodiment of the present invention, the A/U content in the environment of the ribosome binding site of the at least one (m)RNA of the composition of the present invention is increased compared to the A/U content in the environment of the ribosome binding site of its particular wild-type mRNA. This modification (an increased A/U content around the ribosome binding site) increases the efficiency of ribosome binding to the at least one mRNA. An effective binding of the ribosomes to the ribosome binding site (Kozak sequence: GCCGCCACCAUGG (SEQ ID NO: 67), the AUG forms the start codon) in turn has the effect of an efficient translation of the at least one mRNA. According to a further embodiment of the present invention the at least one mRNA of the composition of the present invention may be modified with respect to potentially destabilizing sequence elements. Particularly, the coding region and/or the 5′ and/or 3′ untranslated region of this at least one mRNA may be modified compared to the particular wild type mRNA such that it contains no destabilizing sequence elements, the coded amino acid sequence of the modified at least one mRNA preferably not being modified compared to its particular wild type mRNA. It is known that, for example, in sequences of eukaryotic RNAs destabilizing sequence elements (DSE) occur, to which signal proteins bind and regulate enzymatic degradation of RNA in vivo. For further stabilization of the modified at least one mRNA, optionally in the region which encodes for an antigen, antigenic protein or antigenic peptide as defined herein, one or more such modifications compared to the corresponding region of the wild type mRNA can therefore be carried out, so that no or substantially no destabilizing sequence elements are contained there. According to the invention, DSE present in the untranslated regions (3′- and/or 5′-UTR) can also be eliminated from the at least one mRNA of the composition of the present invention by such modifications. Such destabilizing sequences are e.g. AU-rich sequences (AURES), which occur in 3′-UTR sections of numerous unstable RNAs (Caput et al., Proc. Natl. Acad. Sci. USA 1986, 83: 1670 to 1674). The at least one mRNA of the composition of the present invention is therefore preferably modified compared to the wild type mRNA such that the at least one mRNA contains no such destabilizing sequences. This also applies to those sequence motifs which are recognized by possible endonucleases, e.g. the sequence GAACAAG, which is contained in the 3′-UTR segment of the gene which codes for the transferrin receptor (Binder et al., EMBO J. 1994, 13: 1969 to 1980). These sequence motifs are also preferably removed in the at least one mRNA of the composition of the present invention. Also preferably according to the invention, the at least one mRNA of the composition of the present invention has, in a modified form, at least one IRES as defined above and/or at least one 5′ and/or 3′ stabilizing sequence, in a modified form, e.g. to enhance ribosome binding or to allow expression of different encoded antigens located on an at least one (bi- or even multicistronic) mRNA of the composition of the present invention.

According to the invention, the at least one mRNA of the composition of the present invention furthermore preferably has at least one 5′ and/or 3′ stabilizing sequence. These stabilizing sequences in the 5′ and/or 3′ untranslated regions have the effect of increasing the half-life of the at least one mRNA in the cytosol. These stabilizing sequences can have 100% sequence homology to naturally occurring sequences which occur in viruses, bacteria and eukaryotes, but can also be partly or completely synthetic. The untranslated sequences (UTR) of the β-globin gene, e.g. from Homo sapiens or Xenopus laevis may be mentioned as an example of stabilizing sequences which can be used in the present invention for a stabilized mRNA. Another example of a stabilizing sequence has the general formula (C/U)CCANxCCC(U/A)PyxUC(C/U)CC (SEQ ID NO: 68), which is contained in the 3′UTR of the very stable mRNA which codes for α-globin, α(I)-collagen, 15-lipoxygenase or for tyrosine hydroxylase (cf. Holcik et al., Proc. Natl. Acad. Sci. USA 1997, 94: 2410 to 2414). Such stabilizing sequences can of course be used individually or in combination with one another and also in combination with other stabilizing sequences known to a person skilled in the art. The at least one mRNA of the composition of the present invention is therefore preferably present as globin UTR (untranslated regions)-stabilized mRNA, in particular as α-globin UTR-stabilized mRNA. Preferably the at least one mRNA of the composition comprises a stabilizing sequence in the 3′-UTR derived from the center, α-complex-binding portion of the 3′UTR of an α-globin gene, such as of a human α-globin gene, preferably according to SEQ ID NO: 69:

Center, α-complex-binding portion of the 3′UTR of an α-globin gene (also named herein as “muag”)

(SEQ ID NO: 69)
GCCCGAUGGGCCUCCCAACGGGCCCUCCUCCCCUCCUUGCACCG

Nevertheless, substitutions, additions or eliminations of bases are preferably carried out with the at least one mRNA of the composition of the present invention, using a DNA matrix for preparation of the at least one mRNA of the composition of the present invention by techniques of the well known site directed mutagenesis or with an oligonucleotide ligation strategy (see e.g. Maniatis et al., Molecular Cloning: A Laboratory Manual, Cold Spring Harbor Laboratory Press, 3rd ed., Cold Spring Harbor, N. Y., 2001). In such a process, for preparation of the at least one mRNA, a corresponding DNA molecule may be transcribed in vitro. This DNA matrix preferably comprises a suitable promoter, e.g. a T7 or SP6 promoter, for in vitro transcription, which is followed by the desired nucleotide sequence for the at least one mRNA to be prepared and a termination signal for in vitro transcription. The DNA molecule, which forms the matrix of an at least one mRNA of interest, may be prepared by fermentative proliferation and subsequent isolation as part of a plasmid which can be replicated in bacteria. Plasmids which may be mentioned as suitable for the present invention are e.g. the plasmids pT7 Ts (GenBank accession number U26404; Lai et al., Development 1995, 121: 2349 to 2360), pGEM® series, e.g. pGEM®-1 (GenBank accession number X65300; from Promega) and pSP64 (GenBank accession number X65327); cf. also Mezei and Storts, Purification of PCR Products, in: Griffin and Griffin (ed.), PCR Technology: Current Innovation, CRC Press, Boca Raton, Fla., 2001.

The stabilization of the at least one mRNA of the composition of the present invention can likewise by carried out by associating or complexing the at least one mRNA with, or binding it to, a cationic compound, in particular a polycationic compound, for example a (poly)cationic peptide or protein. In particular the use of protamine, nucleoline, spermin or spermidine as the polycationic, nucleic-acid-binding protein to the mRNA is particularly effective. Furthermore, the use of other cationic peptides or proteins, such as poly-L-lysine or histones, is likewise possible. This procedure for stabilizing RNA is described in EP-A-1083232, the disclosure of which is incorporated by reference into the present invention in its entirety. Further preferred cationic substances which can be used for stabilizing the mRNA of the composition of the present invention include cationic polysaccharides, for example chitosan, polybrene, polyethyleneimine (PEI) or poly-L-lysine (PLL), etc. Association or complexing of the at least one mRNA of the inventive composition with cationic compounds, e.g. cationic proteins or cationic lipids, e.g. oligofectamine as a lipid based complexation reagent) preferably increases the transfer of the at least one mRNA present as a pharmaceutically active component into the cells to be treated or into the organism to be treated. It is also referred to the disclosure herein with regard to the stabilizing effect for the at least one mRNA of the composition of the present invention by complexation, which holds for the stabilization of RNA as well.

According to another particularly preferred embodiment, the at least one mRNA of the composition may additionally or alternatively encode a secretory signal peptide. Such signal peptides are sequences, which typically exhibit a length of about 15 to 30 amino acids and are preferably located at the N-terminus of the encoded peptide, without being limited thereto. Signal peptides as defined herein preferably allow the transport of the antigen, antigenic protein or antigenic peptide as encoded by the at least one mRNA of the composition into a defined cellular compartiment, preferably the cell surface, the endoplasmic reticulum (ER) or the endosomal-lysosomal compartiment. Examples of secretory signal peptide sequences as defined herein include, without being limited thereto, signal sequences of classical or non-classical MHC-molecules (e.g. signal sequences of MHC I and II molecules, e.g. of the MHC class I molecule HLA-A*0201), signal sequences of cytokines or immunoglobulines as defined herein, signal sequences of the invariant chain of immunoglobulines or antibodies as defined herein, signal sequences of Lamp1, Tapasin, Erp57, Calretikulin, Calnexin, and further membrane associated proteins or of proteins associated with the endoplasmic reticulum (ER) or the endosomal-lysosomal compartiment. Particularly preferably, signal sequences of MEW class I molecule HLA-A*0201 may be used according to the present invention.

Any of the above modifications may be applied to the at least one mRNA of the composition of the present invention, and further to any (m)RNA as used in the context of the present invention and may be, if suitable or necessary, be combined with each other in any combination, provided, these combinations of modifications do not interfere with each other in the respective RNA. A person skilled in the art will be able to take his choice accordingly.

According to a preferred embodiment, the composition comprises at least one mRNA that has been modified as described herewithin, which comprises at least one coding sequence selected from RNA sequences being identical or at least 80% identical to the RNA sequence of SEQ ID NOs: 3, 6, 9, 12, 15, 18, 82, 83, 84, or 85. Even more preferably, the composition comprises six mRNAs, wherein the coding sequence in each mRNA is identical or at least 80% identical to one of the RNA sequences according to SEQ ID NOs: 3, 6, 9, 12, 15, 18, 82, 83, 84, or 85.

In a preferred embodiment, each of the six antigens of the composition of the present invention, may be encoded by one (monocistronic) mRNA. In other words, the composition of the present invention may contain six (monocistronic) mRNAs, wherein each of these six (monocistronic) mRNAs, may encode just one antigen as defined above.

In an even more preferred embodiment, the composition comprises six mRNAs, each of which has been modified as described herewithin, wherein one mRNA encodes PSA, one mRNA encodes PSMA, one mRNA encodes PSCA, one mRNA encodes STEAP, one mRNA encodes PAP and one mRNA encodes MUC1 or fragments or variants thereof, respectively.

In an even more preferred embodiment, the composition comprises six mRNAs, wherein one mRNA encodes PSA and comprises a coding sequence identical or at least 80% identical to SEQ ID NO: 3 or 82, one mRNA encodes PSMA and comprises a coding sequence identical or at least 80% identical to SEQ ID NO: 6 or 83, one mRNA encodes PSCA and comprises a coding sequence identical or at least 80% identical to SEQ ID NO: 9 or 84, one mRNA encodes STEAP and comprises a coding sequence identical or at least 80% identical to SEQ ID NO: 12 or 85, one mRNA encodes PAP and comprises a coding sequence identical or at least 80% identical to SEQ ID NO: 15 and one mRNA encodes MUC1 and comprises a coding sequence identical or at least 80% identical to SEQ ID NO: 18 (or fragments or variants of each of these sequences) and optionally further excipients.

In one embodiment, the composition comprises at least one mRNA, which is identical or at least 80% identical to the RNA sequence of SEQ ID NOs: 1, 4, 7, 10, 13 or 16. Even more preferably, the composition comprises six mRNAs, wherein each mRNA is identical or at least 80% identical to one of the RNA sequences according to SEQ ID NOs: 1, 4, 7, 10, 13 or 16.

In an even more preferred embodiment, the composition comprises six mRNAs, wherein one mRNA encodes PSA and is identical or at least 80% identical to SEQ ID NO: 1, one mRNA encodes PSMA and is identical or at least 80% identical to SEQ ID NO: 4, one mRNA encodes PSCA and is identical or at least 80% identical to SEQ ID NO: 7, one mRNA encodes STEAP and is identical or at least 80% identical to SEQ ID NO: 10, one mRNA encodes PAP and is identical or at least 80% identical to SEQ ID NO: 13 and one mRNA encodes MUC1 and is identical or at least 80% identical to SEQ ID NO: 16 (or fragments or variants of each of these sequences) and optionally further excipients.

According to a further preferred embodiment of the invention, the at least one mRNA of the compositions described above comprises a histone stem-loop in the 3′ UTR region. Preferably, the composition comprises six mRNAs, wherein each of the mRNAs comprises a histone stem-loop as defined herewithin.

In a preferred embodiment, the composition comprises six mRNAs, wherein one mRNA encodes PSA and is identical or at least 80% identical to SEQ ID NO: 19, one mRNA encodes PSMA and is identical or at least 80% identical to SEQ ID NO: 20, one mRNA encodes PSCA and is identical or at least 80% identical to SEQ ID NO: 21, one mRNA encodes STEAP and is identical or at least 80% identical to SEQ ID NO: 22, one mRNA encodes PAP and is identical or at least 80% identical to SEQ ID NO: 23 and one mRNA encodes MUC1 and is identical or at least 80% identical to SEQ ID NO: 24 (or fragments or variants of each of these sequences) and optionally further excipients.

According to another embodiment, the composition according to the invention may comprise an adjuvant in order to enhance the immunostimulatory properties of the composition. In this context, an adjuvant may be understood as any compound, which is suitable to support administration and delivery of the composition according to the invention. Furthermore, such an adjuvant may, without being bound thereto, initiate or increase an immune response of the innate immune system, i.e. a non-specific immune response. In other words, when administered, the composition according to the invention typically initiates an adaptive immune response due to the at least six antigens encoded by the at least one mRNA contained in the inventive composition. Additionally, the composition according to the invention may generate an (supportive) innate immune response due to addition of an adjuvant as defined herein to the composition according to the invention.

Such an adjuvant may be selected from any adjuvant known to a skilled person and suitable for the present case, i.e. supporting the induction of an immune response in a mammal. Preferably, the adjuvant may be selected from the group consisting of, without being limited thereto, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MF59™; (squalene-water emulsion); MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURAN™ (□μ-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON™ (QS-21); Quil-A (Quil-A saponin); S-28463 (4-amino-otec-dimethyl-2-ethoxymethyl-1H-imidazo[4,5 c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin, microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide.

Suitable adjuvants may also be selected from cationic or polycationic compounds wherein the adjuvant is preferably prepared upon complexing the at least one mRNA of the inventive composition with the cationic or polycationic compound. Association or complexing the at least one mRNA of the composition with cationic or polycationic compounds as defined herein preferably provides adjuvant properties and confers a stabilizing effect to the at least one mRNA of the composition. Particularly preferredcationic or polycationic compounds are selected from cationic or polycationic peptides or proteins, including protamine, nucleoline, spermin or spermidine, or other cationic peptides or proteins, such as poly-L-lysine (PLL), poly-arginine, basic polypeptides, cell penetrating peptides (CPPs), including HIV-binding peptides, Tat, HIV-1 Tat (HIV), Tat-derived peptides, Penetratin, VP22 derived or analog peptides, HSV VP22 (Herpes simplex), MAP, KALA or protein transduction domains (PTDs, PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, protamine, spermine, spermidine, or histones. Further preferred cationic or polycationic compounds may include cationic polysaccharides, for example chitosan, polybrene, cationic polymers, e.g. polyethyleneimine (PEI), cationic lipids, e.g. DOTMA: 1-(2,3-sioleyloxy)propyl)□-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, DOTAP: dioleoyloxy-3-(trimethylammonio)propane, DC-6-14: O,O-ditetradecanoyl-N-(-trimethylammonioacetyl)diethanolamine chloride, CLIP1: rac-(2,3-dioctadecyloxypropyl)(2-hydroxyethyl)□-dimethyl ammonium chloride, CLIP6: rac-2(2,3-dihexadecyloxypropyl-oxymethyloxy)ethyl□trimethyl ammonium, CLIPS: rac-2(2,3-dihexadecyloxypropyl-oxysuccinyloxy)ethyl□-trimethylammonium, oligofectamine, or cationic or polycationic polymers, e.g. modified polyaminoacids, such as □-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified Amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, Chitosan, etc., silan backbone based polymers, such as PMOXA-PDMS copolymers, etc., Blockpolymers consisting of a combination of one or more cationic blocks (e.g. selected of a cationic polymer as mentioned above) and of one or more hydrophilic- or hydrophobic blocks (e.g polyethyleneglycole); etc.

Additionally, preferred cationic or polycationic proteins or peptides, which can be used as an adjuvant by complexing the at least one mRNA of the composition, may be selected from following proteins or peptides having the following total formula (III): (Arg)l;(Lys)m;(His)n;(Orn)o;(Xaa)x, wherein l+m+n+o+x=8-15, and l, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide. Particularly preferred oligoarginines in this context are e.g. Arg7, Arg8, Arg9, Arg7, H3R9, R9H3, H3R9H3, YSSR9SSY, (RKH)4, Y(RKH)2R, etc.

The ratio of the RNA to the cationic or polycationic compound in the adjuvant component may be calculated on the basis of the nitrogen/phosphate ratio (N/P-ratio) of the entire RNA complex, i.e. the ratio of positively charged (nitrogen) atoms of the cationic or polycationic compound to the negatively charged phosphate atoms of the nucleic acids. For example, 1 μg RNA typically contains about 3 nmol phosphate residues, provided the RNA exhibits a statistical distribution of bases. Additionally, 1 μg peptide typically contains about x nmol nitrogen residues, dependent on the molecular weight and the number of basic amino acids. When exemplarily calculated for (Arg)9 (molecular weight 1424 g/mol, 9 nitrogen atoms), 1 (Arg)9 contains about 700 pmol (Arg)9 and thus 700×9=6300 pmol basic amino acids=6.3 nmol nitrogen atoms. For a mass ratio of about 1:1 RNA/(Arg)9 an N/P ratio of about 2 can be calculated. When exemplarily calculated for protamine (molecular weight about 4250 g/mol, 21 nitrogen atoms, when protamine from salmon is used) with a mass ratio of about 2:1 with 2 μg RNA, 6 nmol phosphate are to be calculated for the RNA; 1 μg protamine contains about 235 pmol protamine molecules and thus 235×21=4935 pmol basic nitrogen atoms=4.9 nmol nitrogen atoms. For a mass ratio of about 2:1 RNA/protamine an N/P ratio of about 0.81 can be calculated. For a mass ratio of about 8:1 RNA/protamine an N/P ratio of about 0.2 can be calculated. In the context of the present invention, an N/P-ratio is preferably in the range of about 0.1-10, preferably in a range of about 0.3-4 and most preferably in a range of about 0.5-2 or 0.7-2 regarding the ratio of RNA:peptide in the complex, and most preferably in the range of about 0.7-1.5.

In a preferred embodiment, the composition is obtained in two separate steps in order to obtain both, an efficient immunostimulatory effect and efficient translation of the at least one mRNA according to the invention. Therein, a so called “adjuvant component” is prepared by complexing—in a first step—the at least one mRNA of the adjuvant component with a cationic or polycationic compound in a specific ratio to form a stable complex. In this context, it is important, that no free cationic or polycationic compound or only a negligibly small amount remains in the adjuvant component after complexing the mRNA. Accordingly, the ratio of the mRNA and the cationic or polycationic compound in the adjuvant component is typically selected in a range that the mRNA is entirely complexed and no free cationic or polycationic compound or only a neclectably small amount remains in the composition. Preferably the ratio of the adjuvant component, i.e. the ratio of the mRNA to the cationic or polycationic compound is selected from a range of about 6:1 (w/w) to about 0.25:1 (w/w), more preferably from about 5:1 (w/w) to about 0.5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w).

According to a preferred embodiment, the at least one mRNA encoding the antigens according to the invention is added in a second step to the complexed mRNA of the adjuvant component in order to form the (immunostimulatory) composition of the invention. Therein, the at least one mRNA of the invention is added as free mRNA, i.e. mRNA, which is not complexed by other compounds. Prior to addition, the at least one free mRNA is not complexed and will preferably not undergo any detectable or significant complexation reaction upon the addition of the adjuvant component. This is due to the strong binding of the cationic or polycationic compound to the above described at least one mRNA in the adjuvant component. In other words, when the at least one free mRNA, encoding at least one of the antigens according to the invention, is added to the “adjuvant component”, preferably no free or substantially no free cationic or polycationic compound is present, which may form a complex with the at least one free mRNA. Accordingly, an efficient translation of the at least one free mRNA of the inventive composition is possible in vivo. Therein, the at least one free mRNA may occur as a mono-, di-, or multicistronic mRNA, i.e. an mRNA which carries the coding sequences of one or more proteins. Such coding sequences in di-, or even multicistronic mRNA may be separated by at least one IRES sequence, e.g. as defined herein.

In a particularly preferred embodiment, the at least one free mRNA, which is comprised in the inventive composition, may be identical or different to the at least one mRNA of the adjuvant component of the inventive composition, depending on the specific requirements of therapy. Even more preferably, the at least one free mRNA, which is comprised in the inventive composition, is identical to the at least one mRNA of the adjuvant component of the inventive immunostimulatory composition.

In a particularly preferred embodiment, the composition comprises at least one mRNA, wherein at least one mRNA is encoding the antigens as defined above and wherein said mRNA is present in the composition partially as free mRNA and partially as complexed mRNA. Preferably, the at least one mRNA encoding one or more antigens as defined above is complexed as described above and the same at least one mRNA is then added as free mRNA, wherein preferably the compound, which is used for complexing the mRNA is not present in free form in the composition at the moment of addition of the free mRNA component.

The ratio of the first component (i.e. the adjuvant component comprising or consisting of the at least one mRNA complexed with a cationic or polycationic compound) and the second component (i.e. the at least one free mRNA) may be selected in the inventive composition according to the specific requirements of a particular therapy. Typically, the ratio of the adjuvant component and the at least one free mRNA (adjuvant component:free RNA) of the inventive composition is selected such that a significant stimulation of the innate immune system is elicited due to the adjuvant component. In parallel, the ratio is selected such that a significant amount of the at least one free mRNA can be provided in vivo leading to an efficient translation and concentration of the expressed protein in vivo, e.g. the antigens as defined above. Preferably the ratio of the mRNA in the adjuvant component:free mRNA in the inventive composition is selected from a range of about 5:1 (w/w) to about 1:10 (w/w), more preferably from a range of about 4:1 (w/w) to about 1:8 (w/w), even more preferably from a range of about 3:1 (w/w) to about 1:5 (w/w) or 1:3 (w/w), and most preferably the ratio of the RNA in the adjuvant component:free mRNA in the inventive composition is selected from a ratio of about 1:1 (w/w).

Additionally or alternatively, the ratio of the first component (i.e. the adjuvant component comprising or consisting of the at least one mRNA complexed with a cationic or polycationic compound) and the second component (i.e. the at least one free mRNA) may be calculated on the basis of the nitrogen/phosphate ratio (N/P-ratio) of the entire mRNA complex. In the context of the present invention, an N/P-ratio is preferably in the range of about 0.1-10, preferably in a range of about 0.3-4 and most preferably in a range of about 0.5-2 or 0.7-2 regarding the ratio of RNA:peptide in the complex, and most preferably in the range of about 0.7-1.5.

Additionally or alternatively, the ratio of the first component (i.e. the adjuvant component comprising or consisting of the at least one mRNA complexed with a cationic or polycationic compound) and the second component (i.e. the at least one free mRNA) may also be selected in the inventive composition on the basis of the molar ratio of both mRNAs to each other, i.e. the mRNA of the adjuvant component, being complexed with a cationic or polycationic compound and the at least one free mRNA of the second component. Typically, the molar ratio of the mRNA of the adjuvant component to the at least one free mRNA of the second component may be selected such, that the molar ratio suffices the above (w/w) and/or N/P-definitions. More preferably, the molar ratio of the mRNA of the adjuvant component to the at least one free mRNA of the second component may be selected e.g. from a molar ratio of about 0.001:1, 0.01:1, 0.1:1, 0.2:1, 0.3:1, 0.4:1, 0.5:1, 0.6:1, 0.7:1, 0.8:1, 0.9:1, 1:1, 1:0.9, 1:0.8, 1:0.7, 1:0.6, 1:0.5, 1:0.4, 1:0.3, 1:0.2, 1:0.1, 1:0.01, 1:0.001, etc. or from any range formed by any two of the above values, e.g. a range selected from about 0.001:1 to 1:0.001, including a range of about 0.01:1 to 1:0.001, 0.1:1 to 1:0.001, 0.2:1 to 1:0.001, 0.3:1 to 1:0.001, 0.4:1 to 1:0.001, 0.5:1 to 1:0.001, 0.6:1 to 1:0.001, 0.7:1 to 1:0.001, 0.8:1 to 1:0.001, 0.9:1 to 1:0.001, 1:1 to 1:0.001, 1:0.9 to 1:0.001, 1:0.8 to 1:0.001, 1:0.7 to 1:0.001, 1:0.6 to 1:0.001, 1:0.5 to 1:0.001, 1:0.4 to 1:0.001, 1:0.3 to 1:0.001, 1:0.2 to 1:0.001, 1:0.1 to 1:0.001, 1:0.01 to 1:0.001, or a range of about 0.01:1 to 1:0.01, 0.1:1 to 1:0.01, 0.2:1 to 1:0.01, 0.3:1 to 1:0.01, 0.4:1 to 1:0.01, 0.5:1 to 1:0.01, 0.6:1 to 1:0.01, 0.7:1 to 1:0.01, 0.8:1 to 1:0.01, 0.9:1 to 1:0.01, 1:1 to 1:0.01, 1:0.9 to 1:0.01, 1:0.8 to 1:0.01, 1:0.7 to 1:0.01, 1:0.6 to 1:0.01, 1:0.5 to 1:0.01, 1:0.4 to 1:0.01, 1:0.3 to 1:0.01, 1:0.2 to 1:0.01, 1:0.1 to 1:0.01, 1:0.01 to 1:0.01, or including a range of about 0.001:1 to 1:0.01, 0.001:1 to 1:0.1, 0.001:1 to 1:0.2, 0.001:1 to 1:0.3, 0.001:1 to 1:0.4, 0.001:1 to 1:0.5, 0.001:1 to 1:0.6, 0.001:1 to 1:0.7, 0.001:1 to 1:0.8, 0.001:1 to 1:0.9, 0.001:1 to 1:1, 0.001 to 0.9:1, 0.001 to 0.8:1, 0.001 to 0.7:1, 0.001 to 0.6:1, 0.001 to 0.5:1, 0.001 to 0.4:1, 0.001 to 0.3:1, 0.001 to 0.2:1, 0.001 to 0.1:1, or a range of about 0.01:1 to 1:0.01, 0.01:1 to 1:0.1, 0.01:1 to 1:0.2, 0.01:1 to 1:0.3, 0.01:1 to 1:0.4, 0.01:1 to 1:0.5, 0.01:1 to 1:0.6, 0.01:1 to 1:0.7, 0.01:1 to 1:0.8, 0.01:1 to 1:0.9, 0.01:1 to 1:1, 0.001 to 0.9:1, 0.001 to 0.8:1, 0.001 to 0.7:1, 0.001 to 0.6:1, 0.001 to 0.5:1, 0.001 to 0.4:1, 0.001 to 0.3:1, 0.001 to 0.2:1, 0.001 to 0.1:1, etc.

Even more preferably, the molar ratio of the mRNA of the adjuvant component to the at least one free mRNA of the second component may be selected e.g. from a range of about 0.01:1 to 1:0.01. Most preferably, the molar ratio of the at least one mRNA of the adjuvant component to the at least one free mRNA of the second component may be selected e.g. from a molar ratio of about 1:1. Any of the above definitions with regard to (w/w) and/or N/P ratio may also apply.

Suitable adjuvants may furthermore be selected from nucleic acids having the formula (IV): GlXmGn, wherein: G is guanosine, uracil or an analogue of guanosine or uracil; X is guanosine, uracil, adenosine, thymidine, cytosine or an analogue of the above-mentioned nucleotides; l is an integer from 1 to 40, wherein when l=1 G is guanosine or an analogue thereof, when l>1 at least 50% of the nucleotides are guanosine or an analogue thereof m is an integer and is at least 3; wherein when m=3 X is uracil or an analogue thereof, when m>3 at least 3 successive uracils or analogues of uracil occur; n is an integer from 1 to 40, wherein when n=1 G is guanosine or an analogue thereof, when n>1 at least 50% of the nucleotides are guanosine or an analogue thereof.

Other suitable adjuvants may furthermore be selected from nucleic acids having the formula (V): ClXmCn, wherein: C is cytosine, uracil or an analogue of cytosine or uracil; X is guanosine, uracil, adenosine, thymidine, cytosine or an analogue of the above-mentioned nucleotides; l is an integer from 1 to 40, wherein when l=1 C is cytosine or an analogue thereof, when l>1 at least 50% of the nucleotides are cytosine or an analogue thereof; m is an integer and is at least 3; wherein when m=3 X is uracil or an analogue thereof, when m>3 at least 3 successive uracils or analogues of uracil occur; n is an integer from 1 to 40, wherein when n=1 C is cytosine or an analogue thereof, when n>1 at least 50% of the nucleotides are cytosine or an analogue thereof.

According to a further aspect the present invention may provide a vaccine which is based on at least one mRNA, preferably at least six distinct mRNA species, encoding at least the above defined antigens PSMA, PSA, PSCA, STEAP, PAP and MUC-1. Accordingly, the inventive vaccine is based on the same components as the composition as defined above. Insofar, it may be referred to the above disclosure defining the inventive composition. The inventive vaccine may, however, be provided in physically separate form and may be administered by separate administration steps. The inventive vaccine may correspond to the inventive composition, if the mRNA components are provided by one single composition. However, the inventive vaccine may e.g. be provided physically separated. E.g., the mRNA species may be provided such that two separate compositions, which may contain at least one mRNA species each (e.g. three distinct mRNA species) encoding three distinct antigens, are provided, which may or may not be combined. Also, the inventive vaccine may be a combination of three distinct compositions, each composition comprising at least one mRNA encoding two of the above six antigens. Or, the vaccine may be provided as a combination of at least one mRNA, preferably six mRNAs, each encoding one of the above defined six antigens. The vaccine may be combined to provide one single composition prior to its use or it may be used such that more than one administration is required to administer the distinct mRNA species coding for the above defined six distinct antigens. If the vaccine contains at least one mRNA molecule, typically at least two, three, four, five or six mRNA molecules, encoding the above defined six antigens, it may e.g. be administered by one single administration (combining all mRNA species), by two separate administrations (e.g. each administration administering mRNA molecules encoding for three of the above six antigens), by three, four, five or six administrations (in case all of the mRNA species encode one of the above defined six antigens and are provided physically separate). Accordingly; any combination of mono-, bi- or multicistronic mRNAs encoding the above defined six antigens (and optionally further antigens), provided as separate entities (containing one mRNA species) or as combined entity (containing more than one mRNA species), is understood as a vaccine according to the present invention. According to a particularly preferred embodiment of the inventive vaccine, each of the antigens according to the invention is provided as an individual (monocistronic) mRNA, which is administered separately.

As the composition according to the present invention, the entities of the vaccine may be provided in liquid and or in dry (e.g. lyophylized) form. They may contain further components, in particular further components allowing for its pharmaceutical use. The inventive vaccine or the inventive composition may, e.g., additionally contain a pharmaceutically acceptable carrier and/or further auxiliary substances and additives and/or adjuvants.

The inventive vaccine or composition typically comprises a safe and effective amount of the at least one mRNA of the composition as defined above encoding the antigens as defined above. As used herein, “safe and effective amount” means an amount of the at least one mRNA of the composition or the vaccine as defined above, that is sufficient to significantly induce a positive modification of prostate cancer (PCa), preferably of prostate adenocarcinoma, locally limited, locally advanced, metastatic, castration-resistant (hormone-refractory), metastatic castration-resistant and non-metastatic castration-resistant prostate cancers and diseases or disorders related thereto. At the same time, however, a “safe and effective amount” is small enough to avoid serious side-effects, that is to say to permit a sensible relationship between advantage and risk. The determination of these limits typically lies within the scope of sensible medical judgment. In relation to the inventive vaccine or composition, the expression “safe and effective amount” preferably means an amount of the mRNA (and thus of the encoded antigens) that is suitable for stimulating the adaptive immune system in such a manner that no excessive or damaging immune reactions are achieved but, preferably, also no such immune reactions below a measurable level. Such a “safe and effective amount” of the at least one mRNA of the composition or vaccine as defined above may furthermore be selected in dependence of the type of mRNA, e.g. monocistronic, bi- or even multicistronic mRNA, since a bi- or even multicistronic mRNA may lead to a significantly higher expression of the encoded antigen(s) than use of an equal amount of a monocistronic RNA. A “safe and effective amount” of the at least one mRNA of the composition or the vaccine as defined above will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The vaccine or composition according to the invention can be used according to the invention for human and also for veterinary medical purposes, as a pharmaceutical composition or as a vaccine.

In a preferred embodiment, the at least one mRNA of the composition, vaccine or kit of parts according to the invention is provided in lyophilized form. Preferably, the at least one lyophilized mRNA is reconstituted in a suitable buffer, advantageously based on an aqueous carrier, prior to administration, e.g. Ringer-Lactate solution, which is preferred, Ringer solution, a phosphate buffer solution. In a preferred embodiment, the composition, the vaccine or the kit of parts according to the invention contains six mRNAs, which are provided separately in lyophilized form (optionally together with at least one further additive) and which are preferably reconstituted separately in a suitable buffer (such as Ringer-Lactate solution) prior to its use so as to allow individual administration of each of the six (monocistronic) mRNAs.

The vaccine or composition according to the invention may typically contain a pharmaceutically acceptable carrier. The expression “pharmaceutically acceptable carrier” as used herein preferably includes the liquid or non-liquid basis of the inventive composition or vaccine. If the inventive composition or vaccine is provided in liquid form, the carrier will be water, typically pyrogen-free water; isotonic saline or buffered (aqueous) solutions, e.g phosphate, citrate etc. buffered solutions. Particularly for injection of the inventive composition or vaccine, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, Na2CO3, NaHCO3, Na2SO4, examples of the optional potassium salts include e.g. KCl, KI, KBr, K2CO3, KHCO3, K2SO4, and examples of calcium salts include e.g. CaCl2, CaI2, CaBr2, CaCO3, CaSO4, Ca(OH)2. Furthermore, organic anions of the aforementioned cations may be contained in the buffer. According to a more preferred embodiment, the buffer suitable for injection purposes as defined above, may contain salts selected from sodium chloride (NaCl), calcium chloride (CaCl2) and optionally potassium chloride (KCl), wherein further anions may be present additional to the chlorides. CaCl2 can also be replaced by another salt like KCl. Typically, the salts in the injection buffer are present in a concentration of at least 50 mM sodium chloride (NaCl), at least 3 mM potassium chloride (KCl) and at least 0.01 mM calcium chloride (CaCl2). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects.

Reference media are e.g. in “in vivo” methods occurring liquids such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

However, one or more compatible solid or liquid fillers or diluents or encapsulating compounds may be used as well, which are suitable for administration to a person. The term “compatible” as used herein means that the constituents of the inventive composition or vaccine are capable of being mixed with the at least one mRNA, in such a manner that no interaction occurs which would substantially reduce the pharmaceutical effectiveness of the inventive composition or vaccine under typical use conditions. Pharmaceutically acceptable carriers, fillers and diluents must, of course, have sufficiently high purity and sufficiently low toxicity to make them suitable for administration to a person to be treated. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The choice of a pharmaceutically acceptable carrier is determined in principle by the manner, in which the inventive composition or vaccine is administered. The inventive composition or vaccine can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections. More preferably, vaccines may be administered by an intradermal, subcutaneous, or intramuscular route, preferably by injection, which may be needle-free and/or needle injection.

In a preferred embodiment the inventive composition or vaccine is administered by jet injection which is one specific form of needle-free injection. “Jet injection”, as used herein, refers to a needle-free injection method, wherein a fluid containing the at least one mRNA, the composition or vaccine according to the invention and, optionally, further suitable excipients is forced through an orifice, thus generating an ultra-fine liquid stream of high pressure that is capable of penetrating mammalian skin and, depending on the injection settings, subcutaneous tissue or muscle tissue. In principle, the liquid stream forms a hole in the skin, through which the liquid stream is pushed into the target tissue. Preferably, jet injection is used for intradermal, subcutaneous or intramuscular injection of the composition or vaccine according to the invention. In a preferred embodiment, jet injection is used for intramuscular injection of the composition or vaccine. In a further preferred embodiment, jet injection is used for intradermal injection of the composition or vaccine.

Compositions/vaccines are therefore preferably formulated in liquid or solid form. The suitable amount of the inventive composition or vaccine to be administered can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or vaccine is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

The inventive vaccine or composition can additionally contain one or more auxiliary substances in order to further increase the immunogenicity. A synergistic action of the at least one mRNA of the composition or vaccine as defined above and of an auxiliary substance, which may be optionally be co-formulated (or separately formulated) with the inventive vaccine or composition as described above, is preferably achieved thereby. Depending on the various types of auxiliary substances, various mechanisms can come into consideration in this respect. For example, compounds that permit the maturation of dendritic cells (DCs), for example lipopolysaccharides, TNF-alpha or CD40 ligand, form a first class of suitable auxiliary substances. In general, it is possible to use as auxiliary substance any agent that influences the immune system in the manner of a “danger signal” (LPS, GP96, etc.) or cytokines, such as GM-CFS, which allow an immune response produced by the immune-stimulating adjuvant according to the invention to be enhanced and/or influenced in a targeted manner. Particularly preferred auxiliary substances are cytokines, such as monokines, lymphokines, interleukins or chemokines, that—additional to induction of the adaptive immune response by the encoded at least six antigens—promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, INF-gamma, GM-CSF, G-CSF, M-CSF, LT-beta or TNF-alpha, growth factors, such as hGH. Preferably, such immunogenicity increasing agents or compounds are provided separately (not co-formulated with the inventive vaccine or composition) and administered individually.

Further additives which may be included in the inventive vaccine or composition are emulsifiers, such as, for example, Tween□; wetting agents, such as, for example, sodium lauryl sulfate; colouring agents; taste-imparting agents, pharmaceutical carriers; tablet-forming agents; stabilizers; antioxidants; preservatives.

The inventive vaccine or composition can also additionally contain any further compound, which is known to be immune-stimulating due to its binding affinity (as ligands) to human Toll-like receptors TLR1, TLR2, TLR3, TLR4, TLR5, TLR6, TLR7, TLR8, TLR9, TLR10, or due to its binding affinity (as ligands) to murine Toll-like receptors TLR1, TLR2, TLR3, TLR4, TLR5, TLR6, TLR7, TLR8, TLR9, TLR10, TLR11, TLR12 or TLR13.

Another class of compounds, which may be added to an inventive vaccine or composition in this context, may be CpG nucleic acids, in particular CpG-RNA or CpG-DNA. A CpG-RNA or CpG-DNA can be a single-stranded CpG-DNA (ss CpG-DNA), a double-stranded CpG-DNA (dsDNA), a single-stranded CpG-RNA (ss CpG-RNA) or a double-stranded CpG-RNA (ds CpG-RNA). The CpG nucleic acid is preferably in the form of CpG-RNA, more preferably in the form of single-stranded CpG-RNA (ss CpG-RNA). The CpG nucleic acid preferably contains at least one or more (mitogenic) cytosine/guanine dinucleotide sequence(s) (CpG motif(s)). According to a first preferred alternative, at least one CpG motif contained in these sequences, that is to say the C (cytosine) and the G (guanine) of the CpG motif, is unmethylated. All further cytosines or guanines optionally contained in these sequences can be either methylated or unmethylated. According to a further preferred alternative, however, the C (cytosine) and the G (guanine) of the CpG motif can also be present in methylated form.

Preferably, the above compounds are formulated and administered separately from the above composition or vaccine (of the invention) containing the at least one mRNA encoding at least the above defined six antigens.

According to a further aspect of the present invention, the inventive composition or the inventive vaccine may be used according to the present invention (for the preparation of a medicament) for the treatment of prostate cancer (PCa), preferably prostate adenocarcinoma, locally limited, locally advanced, metastatic, castration-resistant (hormone-refractory), metastatic castration-resistant and non-metastatic castration-resistant prostate cancers and diseases or disorders related thereto.

According to a further aspect of the present invention, the inventive vaccine or the inventive composition containing the at least one mRNA encoding the antigens as defined herein may be used for the treatment of prostate cancer (PCa), preferably prostate adenocarcinoma, locally limited, locally advanced, metastatic, castration-resistant (hormone-refractory), metastatic castration-resistant and non-metastatic castration-resistant prostate cancers, and diseases or disorders related thereto.

In this context also included in the present invention are methods of treating prostate cancer (PCa), preferably prostate adenocarcinoma, locally limited, locally advanced, metastatic, castration-resistant (hormone-refractory), metastatic castration-resistant and non-metastatic castration-resistant prostate cancers, and diseases or disorders related thereto, by administering to a subject in need thereof a pharmaceutically effective amount of an inventive vaccine, or a pharmaceutically effective amount of an inventive composition. Such a method typically comprises an optional first step of preparing the inventive composition, or the inventive vaccine, and a second step, comprising administering (a pharmaceutically effective amount of) said inventive composition or said inventive vaccine to a patient in need thereof. A subject in need thereof will typically be a male mammal. In the context of the present invention, the mammal is preferably selected from the group comprising, without being limited thereto, e.g. goat, cattle, swine, dog, cat, donkey, monkey, ape, a rodent such as a mouse, hamster, rabbit and, particularly, human, wherein the mammal typically suffers from prostate cancer (PCa), preferably prostate adenocarcinoma, locally limited, locally advanced, metastatic, castration-resistant (hormone-refractory), metastatic castration-resistant and non-metastatic castration-resistant prostate cancers, and diseases or disorders related thereto.

Specifically, the composition or vaccine according to the invention has beneficial effects for the treatment of subjects with castrate-refractory metastatic adenocarcinoma of the prostate with progressive disease. Typically the subjects have undergone surgical castration or androgen suppression therapy (including a gonadotropin-releasing hormone (GNRH) agonist or antagonist). Preferably, subjects are treated with a progressive disease status even after at least one second-line anti-hormonal manipulation (e.g. antiandrogen). More preferably, subjects are selected that have a serum testosterone level of <50 ng/dL or <1.7 nmol/dL. Disease progression may be characterized, for example, by two consecutive rises of PSA, measured at least 1 week apart, resulting at least in a 50% increase over the nadir and a PSA>2 ng/ml. Progression of the disease may also be assessed radiologically by means known in the art.

Furthermore the composition or vaccine according to the invention has beneficial effects for the (neoadjuvant) treatment of subjects suffering from prostate cancer prior and subsequent of prostatectomy.

The invention relates also to the use of the inventive composition or the at least one mRNA encoding the antigens as defined herein (for the preparation of an inventive vaccine), preferably for eliciting an immune response in a mammal, preferably for the treatment of prostate cancer (PCa), more preferably of prostate adenocarcinoma, locally limited, locally advanced, metastatic, castration-resistant (hormone-refractory), metastatic castration-resistant and non-metastatic castration-resistant prostate cancers, and diseases or disorders related thereto.

Similarly, the invention also relates to the use of the inventive vaccine per se or the at least one mRNA encoding the antigens as defined herein for eliciting an adaptive immune response in a mammal, preferably for the treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto.

Prevention or treatment of prostate adenocarcinoma, locally limited, locally advanced, metastatic, castration-resistant (hormone-refractory), metastatic castration-resistant and non-metastatic castration-resistant prostate cancers and/or hormone-refractory prostate cancers, and diseases or disorders related thereto, may be carried out by administering the combination of antigens according to the invention, either in the form of the inventive composition or in the form of the inventive vaccine in order to elicite an immune response. The immunization protocol for the immunization of a subject against the combination of at least six antigens as defined herein typically comprises a series of single doses or dosages of the inventive composition or the inventive vaccine. A single dosage, as used herein, refers to the initial/first dose, a second dose or any further doses, respectively, which are preferably administered in order to “boost” the immune reaction. In this context, each single dosage comprises the administration of all of the at least six antigens according to the invention, wherein the interval between the administration of two single dosages can vary from at least one day, preferably 2, 3, 4, 5, 6 or 7 days, to at least one week, preferably 2, 3, 4, 5, 6, 7 or 8 weeks. The intervals between single dosages may be constant or vary over the course of the immunization protocol, e.g. the intervals may be shorter in the beginning and longer towards the end of the protocol. Depending on the total number of single dosages and the interval between single dosages, the immunization protocol may extend over a period of time, which preferably lasts at least one week, more preferably several weeks (e.g. 2, 3, 4, 5, 6, 7, 8, 9, 10, 11 or 12 weeks), even more preferably several months (e.g. 3, 4, 5, 6, 7, 8, 9, 10, 11, 12, 18 or 24 months). Each single dosage encompasses the administration of all of the at least six antigens as defined herein and may therefore involve at least one, preferably 1, 2, 3, 4, 5, 6, 7, 8, 9, 10, 11 or 12 injections. In the case, where the composition according to the invention is administered, a single dosage typically comprises one injection. In the case, where the vaccine comprises separate mRNA formulations encoding the respective antigens according to the invention, the minimum number of injections carried out during the administration of a single dosage corresponds to the number of separate components of the vaccine. In certain embodiments, the administration of a single dosage may encompass more than one injection for each component of the vaccine (e.g. a specific mRNA formulation comprising a mRNA encoding, for instance, one of the six antigens according to the invention). For example, parts of the total volume of an individual component of the vaccine may be injected into different body parts, thus involving more than one injection. In a more specific example, a single dosage of a vaccine comprising six separate mRNA formulations, each of which is administered in two different body parts, comprises twelve injections. Typically, a single dosage comprises all injections required to administer all components of the vaccine, wherein a single component may be involve more than one injection as outlined above. In the case, where the administration of a single dosage of the vaccine according to the invention encompasses more than one injection, the injection are carried out essentially simultaneously or concurrently, i.e. typically in a time-staggered fashion within the time-frame that is required for the practitioner to carry out the single injection steps, one after the other. The administration of a single dosage therefore preferably extends over a time period of several minutes, e.g. 2, 3, 4, 5, 10, 15, 30 or 60 minutes.

Prevention or treatment of prostate adenocarcinoma, locally limited, locally advanced, metastatic, castration-resistant (hormone-refractory), metastatic castration-resistant and non-metastatic castration-resistant prostate cancers and/or hormone-refractory prostate cancers, and diseases or disorders related thereto, may be carried out by administering the combination of antigens according to the invention, either in the form of the inventive composition or in the form of the inventive vaccine, concurrently, i.e. at once or in a time staggered manner, e.g. as a kit of parts, each part containing at least one mRNA preferably encoding different antigens. Preferably, each of the antigens is administered separately, i.e. each antigen is administered to a different part or region of the body of the subject to be treated, preferably simultaneously or within the same short time-frame, respectively. In a preferred embodiment, the individual mRNAs are administered distributed over the subject's four limbs (i.e. left/right arm and leg). Preferably, the administration (of all at least one mRNAs) occurs within an hour, more preferably within 30 minutes, even more preferably within 15, 10, 5, 4, 3, or 2 minutes or even within 1 minute.

For administration, preferably any of the administration routes may be used as defined above. E.g., one may treat prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto, by inducing or enhancing an adaptive immune response on the basis of the at least six (specifically selected) antigens encoded by the at least one mRNA of the inventive composition. Administering of the inventive composition and/or the inventive vaccine may occur prior, concurrent and/or subsequent to administering another inventive composition and/or inventive vaccine as defined herein which may—in addition—contain another combination of mRNAs encoding different antigens, wherein each antigen encoded by the at least one mRNA of the inventive composition may preferably be suitable for the therapy of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto. In this context, a therapy as defined herein may also comprise the modulation of a disease associated to prostate cancer (PCa), preferably of prostate adenocarcinoma, locally limited, locally advanced, metastatic, castration-resistant (hormone-refractory), metastatic castration-resistant and non-metastatic castration-resistant prostate cancers, and of diseases or disorders related thereto.

According to one further embodiment, the present invention furthermore comprises the use of the inventive composition or the at least one mRNA encoding the antigens as defined herein (for the preparation of an (inventive) vaccine) for modulating, preferably to induce or enhance, an immune response in a mammal as defined above, more preferably to treat and/or to support the treatment of prostate cancer (PCa), preferably of a prostate adenocarcinoma, locally limited, locally advanced, metastatic, castration-resistant (hormone-refractory), metastatic castration-resistant and non-metastatic castration-resistant prostate cancers, or of diseases or disorders related thereto. In this context, the treatment of prostate cancer (PCa) according to the present invention may be assisted by any approach or any combination approaches known from conventional prostate cancer therapy, such as surgery, radiation therapy, hormonal therapy, occasionally chemotherapy, proton therapy, or any combination thereof, and a therapy using the inventive composition as defined herein. Support of the treatment of prostate cancer (PCa) may be also envisaged in any of the other embodiments defined herein. Accordingly, any use of the inventive composition or vaccine in co-therapy with any of the above therapy approaches, in particular in combination with prostate surgery, hormonal (e.g. antiandrogen), and/or chemotherapy is within the scope of the present invention.

Administration of the inventive composition or the at least one mRNA encoding the antigens as defined herein or the inventive vaccine may be carried out in a time staggered treatment. A time staggered treatment may be e.g. administration of the inventive composition or the at least one mRNA encoding the antigens as defined herein or the inventive vaccine prior, concurrent and/or subsequent to a conventional therapy of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto, e.g. by administration of the inventive medicament or the inventive composition or vaccine prior, concurrent and/or subsequent to a therapy or an administration of a therapeutic suitable for the treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto. Such time staggered treatment may be carried out using e.g. a kit, preferably a kit of parts as defined below.

In this context it is particularly preferred that the administration of the inventive composition or inventive vaccine is carried out prior and optional additionally subsequent to prostatectomy (neoadjuvant treatment).

Time staggered treatment may additionally or alternatively also comprise an administration of the inventive composition or vaccine, preferably of the at least one mRNA encoding the antigens as defined above, in a form, wherein the at least one mRNA encoding the antigens as defined above, preferably forming part of the inventive composition or vaccine, is administered parallel, prior or subsequent to another at least one mRNA encoding the antigens as defined above, preferably forming part of the same inventive composition or vaccine. Preferably, the administration (of all at least one mRNAs) occurs within an hour, more preferably within 30 minutes, even more preferably within 15, 10, 5, 4, 3, or 2 minutes or even within 1 minute. Such time staggered treatment may be carried out using e.g. a kit, preferably a kit of parts as defined below.

In a preferred embodiment, the inventive composition or vaccine is administered repeatedly, wherein each administration preferably comprises individual administration of the at least one mRNA according to the invention. At each time point of administration, the at least one mRNA may be administered more than once (e.g. 2 or 3 times). In a particularly preferred embodiment of the invention, six mRNAs (each encoding one of the antigens as defined above) are administered at each time point, wherein each mRNA is administered twice by injection, thus resulting in twelve injections distributed over the four limbs.

According to a final embodiment, the present invention also provides kits, particularly kits of parts, comprising the inventive composition, and/or the inventive vaccine, optionally a liquid vehicle for solubilising and optionally technical instructions with information on the administration and dosage of the inventive composition and/or the inventive vaccine. The technical instructions may contain information about administration and dosage of the inventive composition, and/or the inventive vaccine. Such kits, preferably kits of parts, may be applied e.g. for any of the above mentioned applications or uses, preferably for the use of at least one inventive composition (for the preparation of an inventive medicament, preferably a vaccine) for the treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto. The kits may also be applied for the use of at least one inventive composition (for the preparation of an inventive vaccine) for the treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto, wherein the inventive composition) and/or the vaccine due to the encoded at least six antigens may be capable to induce or enhance an immune response in a mammal as defined above. Such kits may further be applied for the use of at least one inventive composition, (for the preparation of an inventive medicament, preferably a vaccine) for modulating, preferably for eliciting, e.g. to induce or enhance, an immune response in a mammal as defined above, and preferably to support treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto. Kits of parts, as a special form of kits, may contain one or more identical or different inventive compositions and/or one or more identical or different inventive vaccines in different parts of the kit. Kits of parts may also contain an (e.g. one) inventive composition, an (e.g. one) inventive vaccine and/or the at least one mRNA encoding the antigens as defined above in different parts of the kit, e.g. each part of the kit containing at least one mRNA encoding a preferably different antigen. Additionally, a combination of both types of kits of parts is possible. Kits of parts may be used, e.g. when a time staggered treatment is envisaged, e.g. when using different formulations and/or increasing concentrations of the inventive composition, the inventive vaccine and/or the at least one mRNA encoding the antigens as defined above during the same treatment in vivo. Kits of parts may also be used when a separated formulation or administration of at least one of the antigens of the inventive composition (i.e. in parts) is envisaged or necessary (e.g. for technical reasons), but e.g. a combined presence of the different antigens in vivo is still to be achieved. Particularly kits of parts as a special form of kits are envisaged, wherein each part of the kit contains at least one preferably different antigen as defined above, all parts of the kit of parts forming the inventive composition or the inventive vaccine as defined herein. Such specific kits of parts may particularly be suitable, e.g. if different antigens are formulated separately as different parts of the kits, but are then administered at once together or in a time staggered manner to the mammal in need thereof. In the latter case administration of all of the different parts of such a kit typically occurs within a short time limit, such that all antigens are present in the mammal at about the same time subsequent to administration of the last part of the kit. In a preferred embodiment, the kit contains at least two parts containing the six mRNAs according to the invention. Preferably, all six mRNAs are provided in separate parts of the kit, wherein the mRNAs are preferably lyophilised. More preferably, the kit further contains as a part a vehicle for solubilising the at least one mRNA, the vehicle preferably being Ringer-lactate solution. Any of the above kits may be used in a treatment as defined above.

### Advantages of the Present Invention

The present invention provides a composition for the treatment of prostate cancer (PCa), wherein the composition comprises at least one mRNA, encoding at least six antigens capable of eliciting an (adaptive) immune response in a mammal wherein the antigens are selected from the group consisting of PSA (Prostate-Specific Antigen), PSMA (Prostate-Specific Membrane Antigen), PSCA (Prostate Stem Cell Antigen), STEAP (Six Transmembrane Epithelial Antigen of the Prostate), PAP (Prostate Alkaline Phosphatase) and MUC1 (Mucin 1). Such a composition allows efficient treatment of prostate cancer (PCa) or supplementary treatment when using conventional therapies. It furthermore avoids the problem of uncontrolled propagation of the introduced DNA sequences by the use of mRNA as an approach for curative methods. mRNA as used in the inventive composition has additional considerable advantages over DNA expression systems e.g. in immune response, immunization or vaccination. These advantages include, inter alia, that mRNA introduced into a cell is not integrated into the genome. This avoids the risk of mutation of this gene, which otherwise may be completely or partially inactivated or give rise to misinformation. It further avoids other risks of using DNA as an agent to induce an immune response (e.g. as a vaccine) such as the induction of pathogenic anti-DNA antibodies in the patient into whom the foreign DNA has been introduced, so bringing about a (possibly fatal) immune response. In contrast, no anti-RNA antibodies have yet been detected.

### BRIEF DESCRIPTION OF THE DRAWINGS

The following Figures are intended to illustrate the invention further. They are not intended to limit the subject matter of the invention thereto.

FIG. 1: depicts the mRNA sequence KLK3 (GC)-muag-A64-C30 (SEQ ID NO: 1), encoding PSA (prostate specific antigen) (=KLK3). The mRNA contains the following sequence elements:
a GC-optimized sequence for stabilization and a better codon usage
˜64 Adenosin at the 3′-terminal end (poly-A-tail), ˜30 Cytosin at the 3′-terminal end (poly-C-tail).

FIG. 2: depicts the wild type-coding sequence encoding PSA (prostate specific antigen) (=KLK3) according to SEQ ID NO: 2, i.e. the coding sequence (CDS) encoding PSA (prostate specific antigen) without GC-optimized sequence.

FIG. 3: depicts the GC-optimized coding sequence encoding PSA (prostate specific antigen) (=KLK3) according to SEQ ID NO: 3.

FIG. 4: depicts the mRNA sequence FOLH1 (GC)-muag-A64-C30 (SEQ ID NO: 4), encoding PSMA (prostate specific membrane antigen) (=FOLH1). The mRNA contains following sequence elements:
a GC-optimized sequence for stabilization and a better codon usage
˜64 Adenosin at the 3′-terminal end (poly-A-tail), 30 Cytosin at the 3′-terminal end (poly-C-tail).

FIG. 5: depicts the wild type coding sequence encoding PSMA (prostate specific membrane antigen) (=FOLH1) according to SEQ ID NO: 5, i.e. the coding sequence (CDS) encoding PSMA (prostate specific membrane antigen) (=FOLH1) without GC-optimized sequence.

FIG. 6: depicts the GC-optimized coding sequence encoding PSMA (prostate specific membrane antigen) (=FOLH1) according to SEQ ID NO: 6.

FIG. 7: depicts the mRNA sequence PSCA (GC)-muag-A64-C30 (SEQ ID NO: 7), encoding PSCA (prostate stem cell antigen). The mRNA contains following sequence elements:
a GC-optimized sequence for stabilization and a better codon usage
˜64 Adenosin at the 3′-terminal end (poly-A-tail), 30 Cytosin at the 3′-terminal end (poly-C-tail).

FIG. 8: depicts the wild type coding sequence encoding PSCA (prostate stem cell antigen) according to SEQ ID NO: 8, i.e. the coding sequence (CDS) encoding PSCA (prostate stem cell antigen) without GC-optimized sequence.

FIG. 9: depicts the GC-optimized coding sequence encoding PSCA (prostate stem cell antigen) according to SEQ ID NO: 9.

FIG. 10: depicts the mRNA sequence STEAP (GC)-muag-A64-C30 (SEQ ID NO: 10), encoding for STEAP (Six Transmembrane Epithelial Antigen of the Prostate). The mRNA contains following sequence elements:
a GC-optimized sequence for stabilization and a better codon usage
˜64 Adenosin at the 3′-terminal end (poly-A-tail), 30 Cytosin at the 3′-terminal end (poly-C-tail).

FIG. 11: depicts the wild type coding sequence encoding STEAP (Six Transmembrane Epithelial Antigen of the Prostate) according to SEQ ID NO: 11, i.e. the coding sequence (CDS) encoding STEAP (Six Transmembrane Epithelial Antigen of the Prostate) without GC-optimized sequence.

FIG. 12: depicts the GC-optimized coding sequence encoding STEAP (Six Transmembrane Epithelial Antigen of the Prostate) according to SEQ ID NO: 12.

FIG. 13: depicts the mRNA sequence PAP (GC)-muag-A64-C30 (SEQ ID NO: 13), encoding for PAP (Prostate Alkaline Phosphatase). The mRNA contains following sequence elements:
a GC-optimized sequence for stabilization and a better codon usage
˜64 Adenosin at the 3′-terminal end (poly-A-tail), 30 Cytosin at the 3′-terminal end (poly-C-tail).

FIG. 14: depicts the wild type coding sequence encoding PAP (prostate alkaline phosphatase) according to SEQ ID NO: 14, i.e. the coding sequence (CDS) encoding PAP (prostate alkaline phosphatase) without GC-optimized sequence.

FIG. 15: depicts the GC-optimized coding sequence encoding PAP (prostate alkaline phosphatase) according to SEQ ID NO: 15).

FIG. 16: depicts the mRNA sequence RNActive MUC1 5×VNTR (GC)-muag-A64-C30 (SEQ ID NO: 16; R1715), encoding for MUC1 (Mucin 1). The mRNA contains following sequence elements:
a GC-optimized sequence for stabilization and a better codon usage
˜64 Adenosin at the 3′-terminal end (poly-A-tail), 30 Cytosin at the 3′-terminal end (poly-C-tail).

FIG. 17: depicts the wild type coding sequence encoding MUC1 (Mucin 1) according to SEQ ID NO: 17, i.e. the coding sequence (CDS) encoding MUC1 (Mucin 1) without GC-optimized sequence.

FIG. 18: depicts the GC-optimized coding sequence encoding MUC1 (Mucin 1) according to SEQ ID NO: 18.

FIG. 19: depicts the mRNA sequence PSA (GC)-muag-A64-C30-histoneSL (SEQ ID NO: 19). The mRNA contains following sequence elements:
A 5′-CAP, a GC-optimized coding sequence for stabilization and a better codon usage encoding PSA according to SEQ ID NO: 3, the stabilizing sequence “muag” in the 3′-UTR according to SEQ ID No.69, ˜64 Adenosin at the 3′-terminal end (poly-A-tail), ˜30 Cytosin at the 3′-terminal end (poly-C-tail); and a histone stem-loop sequence according to SEQ ID No. 71.

FIG. 20: depicts the mRNA sequence PSMA (GC)-muag-A64-C30-histoneSL (SEQ ID NO: 20). The mRNA contains following sequence elements:
a GC-optimized coding sequence for stabilization and a better codon usage encoding PSMA according to SEQ ID NO: 6, the stabilizing sequence “muag” in the 3′-UTR according to SEQ ID No.69, ˜64 Adenosin at the 3′-terminal end (poly-A-tail), ˜30 Cytosin at the 3′-terminal end (poly-C-tail); and a histone stem-loop sequence according to SEQ ID No. 71.

FIG. 21: depicts the mRNA sequence CAP-PSCA (GC)-muag-A64-C30-histoneSL (SEQ ID NO: 21). The mRNA contains following sequence elements:
a GC-optimized coding sequence for stabilization and a better codon usage encoding PSCA according to SEQ ID NO: 9, the stabilizing sequence “muag” in the 3′-UTR according to SEQ ID No.69, ˜64 Adenosin at the 3′-terminal end (poly-A-tail), ˜30 Cytosin at the 3′-terminal end (poly-C-tail); and a histone stem-loop sequence according to SEQ ID No. 71.

FIG. 22: depicts the mRNA sequence STEAP1 (GC)-muag-A64-C30-histoneSL (SEQ ID NO: 22). The mRNA contains following sequence elements:
a GC-optimized coding sequence for stabilization and a better codon usage encoding STEAP according to SEQ ID NO: 12, the stabilizing sequence “muag” in the 3′-UTR according to SEQ ID No.69, ˜64 Adenosin at the 3′-terminal end (poly-A-tail), ˜30 Cytosin at the 3′-terminal end (poly-C-tail); and a histone stem-loop sequence according to SEQ ID No. 71.

FIG. 23: depicts the mRNA sequence PAP (GC)-muag-A64-C30-histoneSL (R2251) (SEQ ID NO: 23). The mRNA contains following sequence elements:
A 5′-CAP, a GC-optimized coding sequence for stabilization and a better codon usage encoding PAP according to SEQ ID NO: 15, the stabilizing sequence “muag” in the 3′-UTR according to SEQ ID No.69, ˜64 Adenosin at the 3′-terminal end (poly-A-tail), ˜30 Cytosin at the 3′-terminal end (poly-C-tail); and a histone stem-loop sequence according to SEQ ID No. 71.

FIG. 24: depicts the mRNA sequence CAP-MUC1 5×VNTR (GC)-muag-A64-C30-histoneSL (R2312) (SEQ ID NO: 24). The mRNA contains following sequence elements:
a GC-optimized coding sequence for stabilization and a better codon usage encoding MUC1 according to SEQ ID NO: 18, the stabilizing sequence “muag” in the 3′-UTR according to SEQ ID No.69, ˜64 Adenosin at the 3′-terminal end (poly-A-tail), ˜30 Cytosin at the 3′-terminal end (poly-C-tail); and a histone stem-loop sequence according to SEQ ID No. 71.

FIG. 25: shows detection of a PAP-specific cellular immune response by ELISPOT. C57BL/6 mice were vaccinated with 32 μg PAP-RNActive® (PAP (GC)-muag-A64-C30; SEQ ID NO: 13) on days 1, 5, 8, 12 and 15. Ex vivo ELISpot analysis of the secretion of IFN-gamma in splenocytes from vaccinated and control mice was performed on day 5 after last vaccination. Cells were stimulated on the plate either with PAP-derived peptide (predicted MHC-class I epitope) or with control peptide. The graph shows single data points for individual mice.

FIG. 26: shows detection of a MUC1-specific cellular immune response by ELISPOT. C57BL/6 mice were vaccinated with 32 μg MUC1-RNActive® (MUC1 5×VNTR (GC)-muag-A64-C30; SEQ ID NO: 16; R1715) on days 1, 5, 8, 12 and 15. Ex vivo ELISpot analysis of the secretion of IFN-gamma in splenocytes from vaccinated and control mice was performed on day 6 after last vaccination. Cells were stimulated on the plate either with MUC1-derived peptide (predicted MHC-class I epitope) or with control peptide. The graph shows single data points for individual mice.

FIG. 27: depicts the GC-optimized coding sequence encoding PSA (prostate specific antigen) (=KLK3) according to SEQ ID NO: 82.

FIG. 28: depicts the GC-optimized coding sequence encoding PSMA (prostate specific membrane antigen) (=FOLH1) according to SEQ ID NO: 83.

FIG. 29: depicts the GC-optimized coding sequence encoding PSCA (prostate stem cell antigen) according to SEQ ID NO: 84.

FIG. 30: depicts the GC-optimized coding sequence encoding STEAP (Six Transmembrane Epithelial Antigen of the Prostate) according to SEQ ID NO: 85.

FIG. 31: shows the protein sequence of PSA NP_001639.1 according to SEQ ID NO: 76.

FIG. 32: shows the protein sequence of PSMA (FOLH1) NP_004467.1 according to SEQ ID NO: 77.

FIG. 33: shows the protein sequence of PSCA O43653.1 according to SEQ ID NO: 78.

FIG. 34: shows the protein sequence of STEAP NP_036581.1 according to SEQ ID NO: 79.

FIG. 35: shows the protein sequence of PAP NP_001090.2 according to SEQ ID NO: 80.

FIG. 36: shows the protein sequence of MUC1 as deposited under accession number AAA60019.1 (J05582.1) according to SEQ ID NO: 81

FIG. 37: shows the protein sequence of MUC1 5×VNTR according to SEQ ID NO: 86

FIG. 38: depicts the wildtype coding sequence encoding MUC1 according to SEQ ID NO: 87, i.e. the full-length coding sequence (CDS) encoding MUC1 without GC-optimized coding sequence.

### EXAMPLES

The following examples are intended to illustrate the invention further. They are not intended to limit the subject matter of the invention thereto.

### 1. Preparation of Encoding Plasmids:

In the following experiment DNA sequences, corresponding to the respective mRNA sequences end encoding the antigens
PSA (Prostate-Specific Antigen),
PSMA (Prostate-Specific Membrane Antigen),
PSCA (Prostate Stem Cell Antigen),
STEAP (Six Transmembrane Epithelial Antigen of the Prostate),
PAP (Prostate Alkaline Phosphatase) and
MUC1 (Mucin 1),
   respectively, were prepared and used for in vitro transcription and transfection experiments.

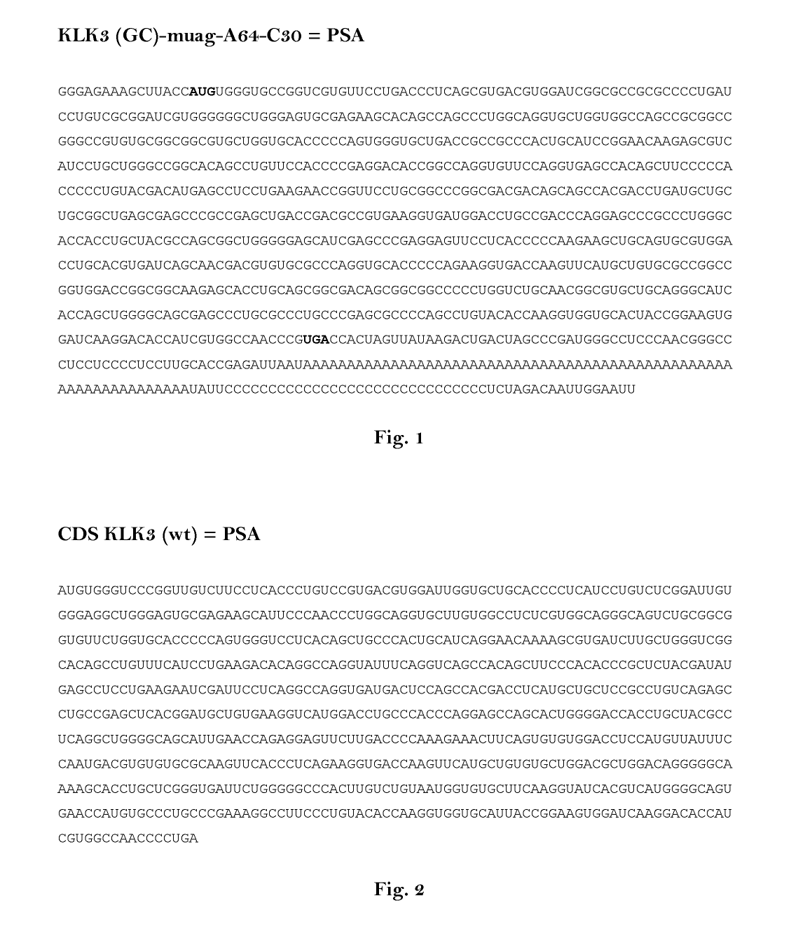

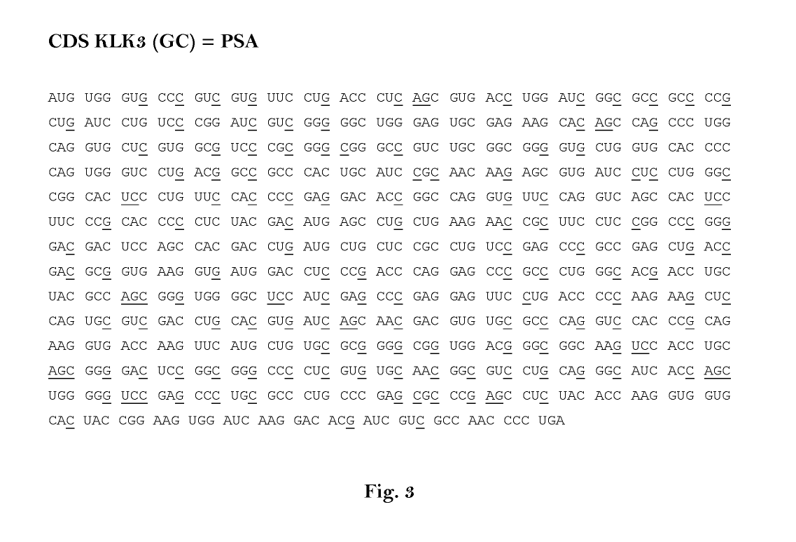

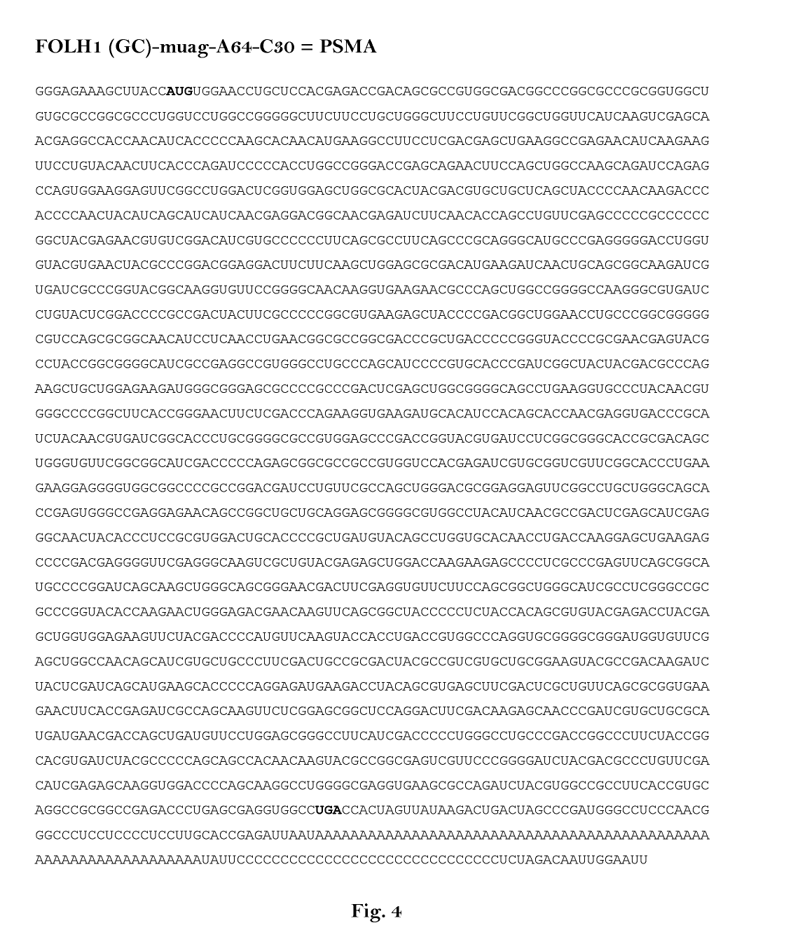

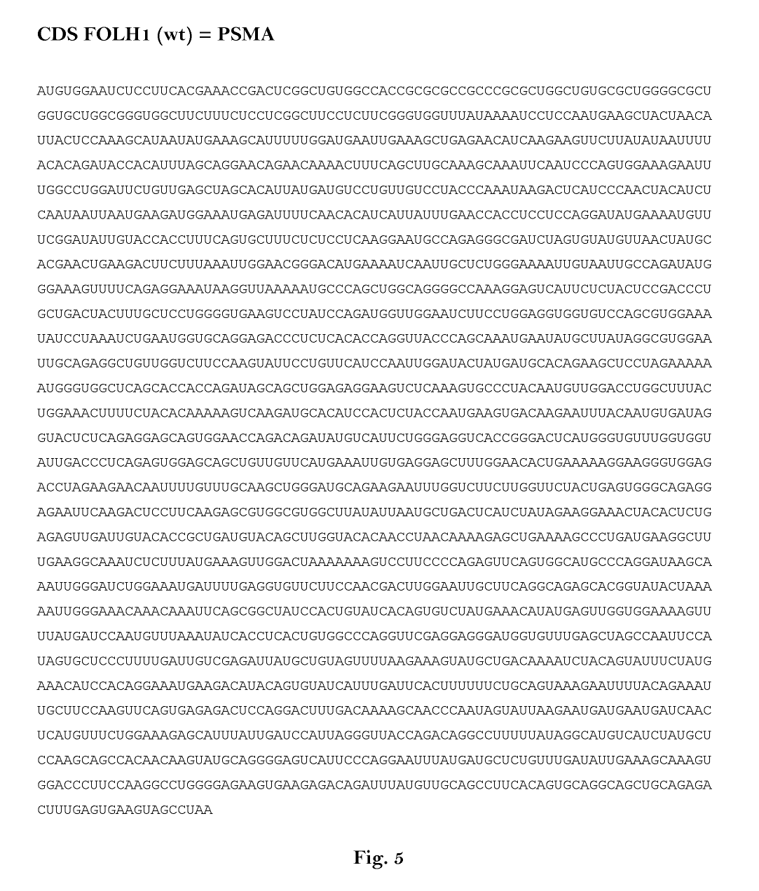

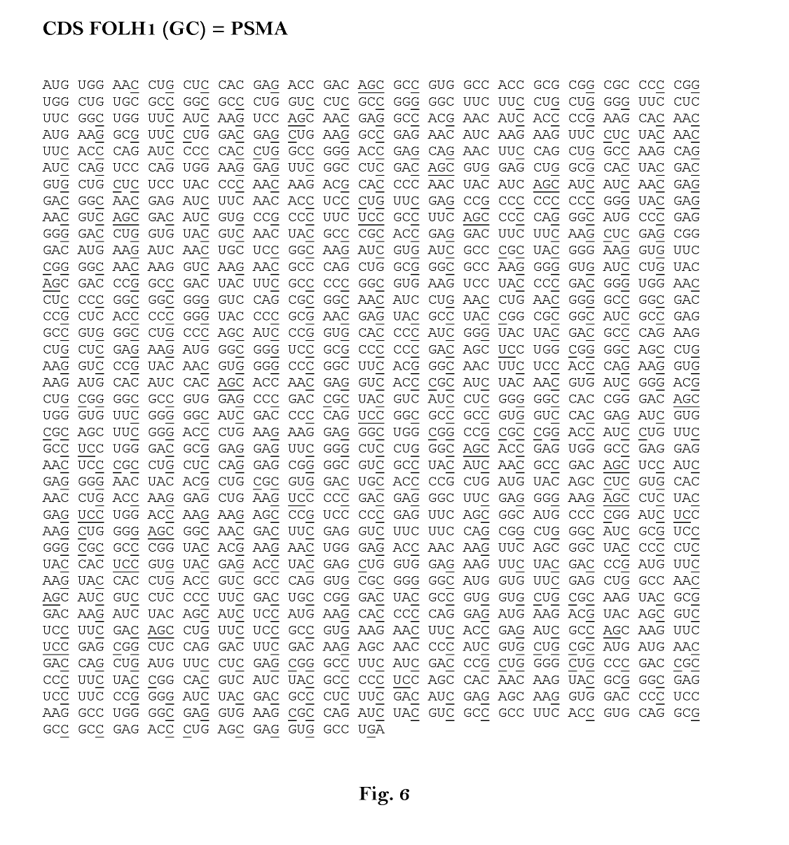

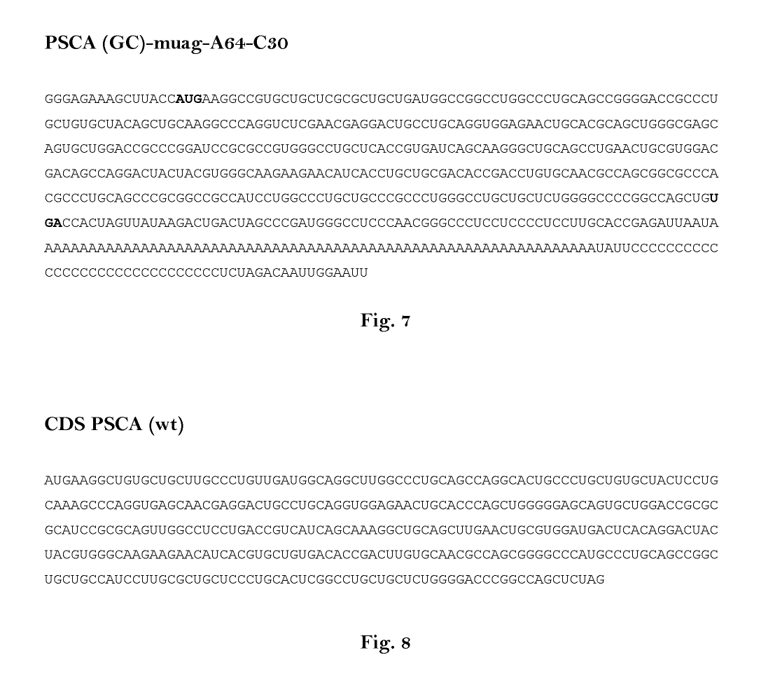

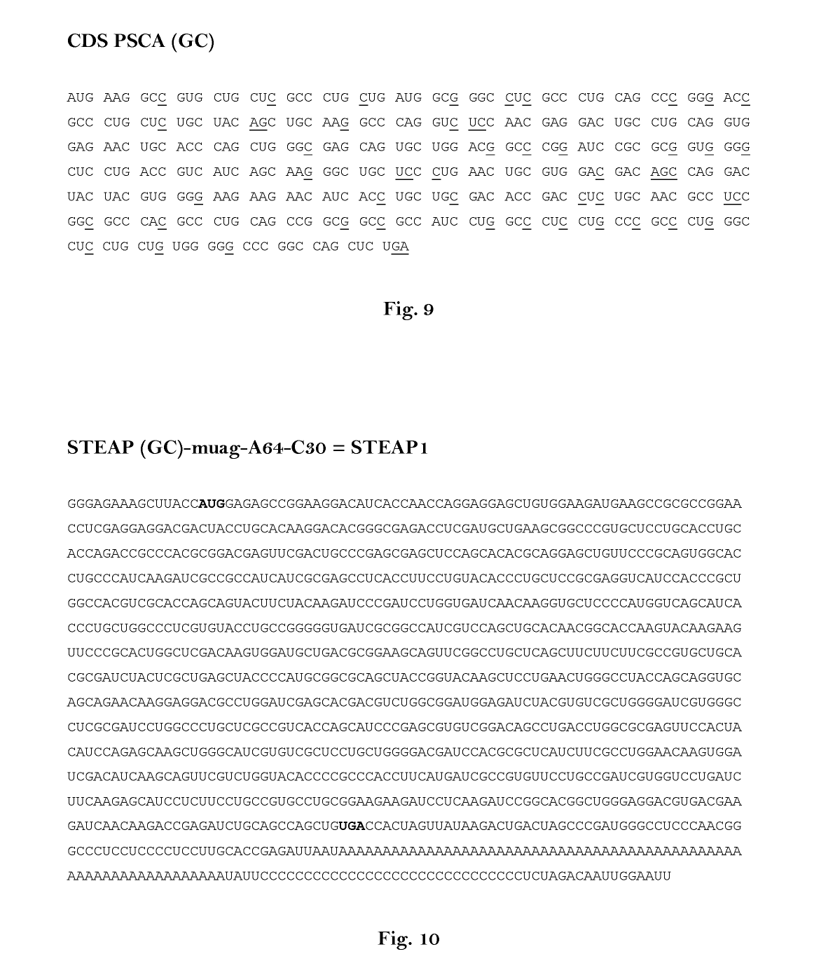

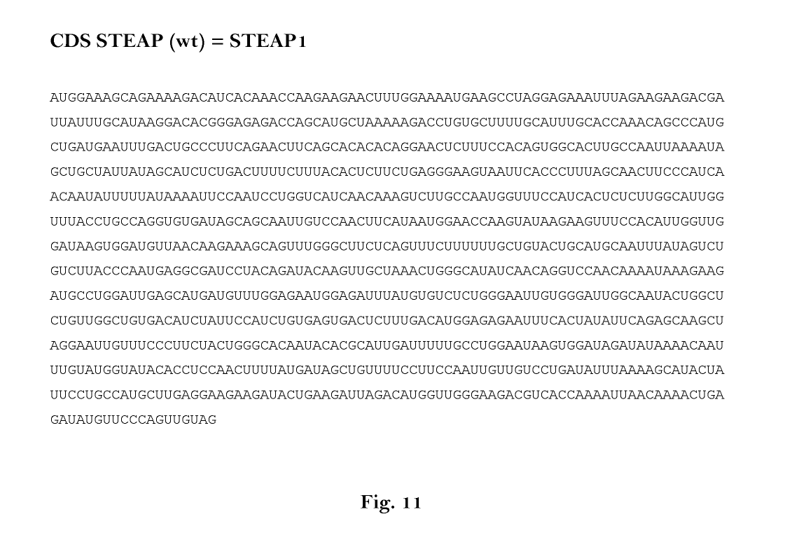

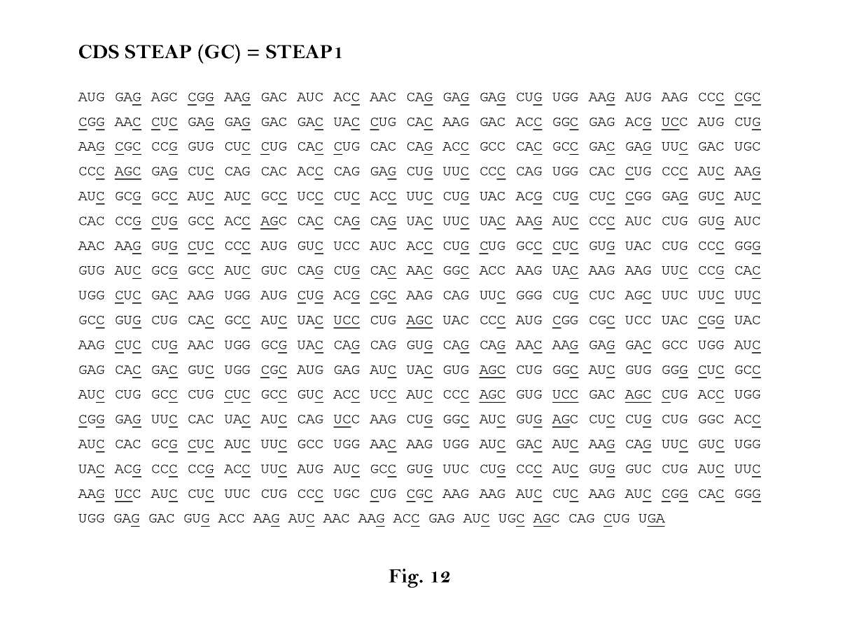

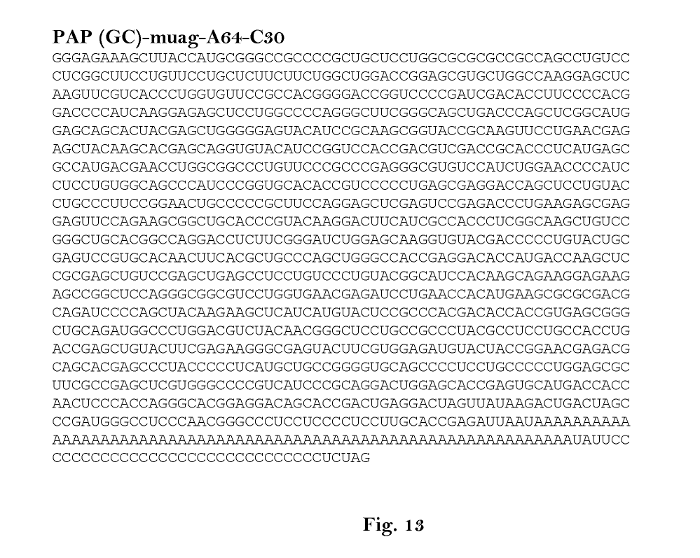

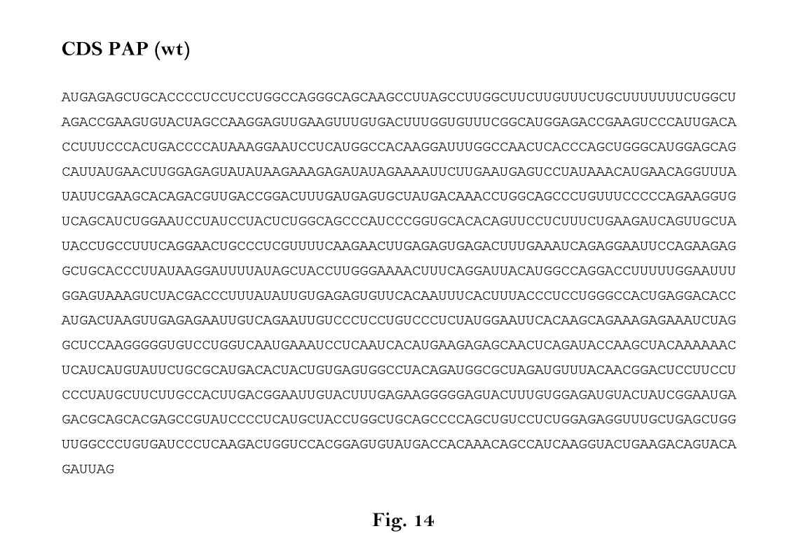

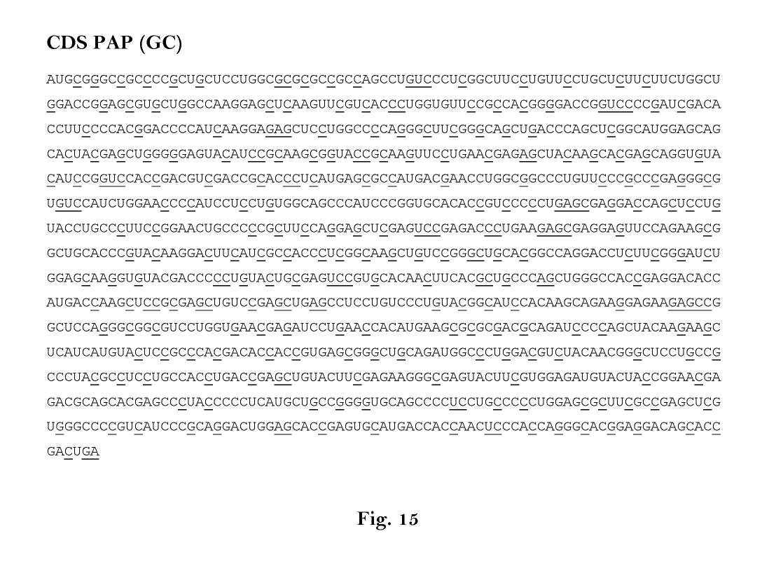

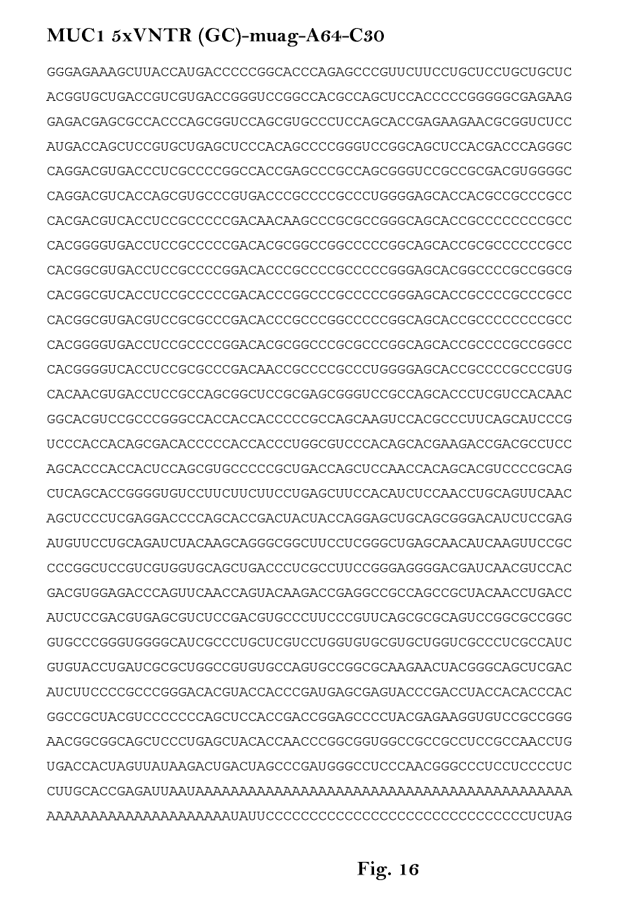

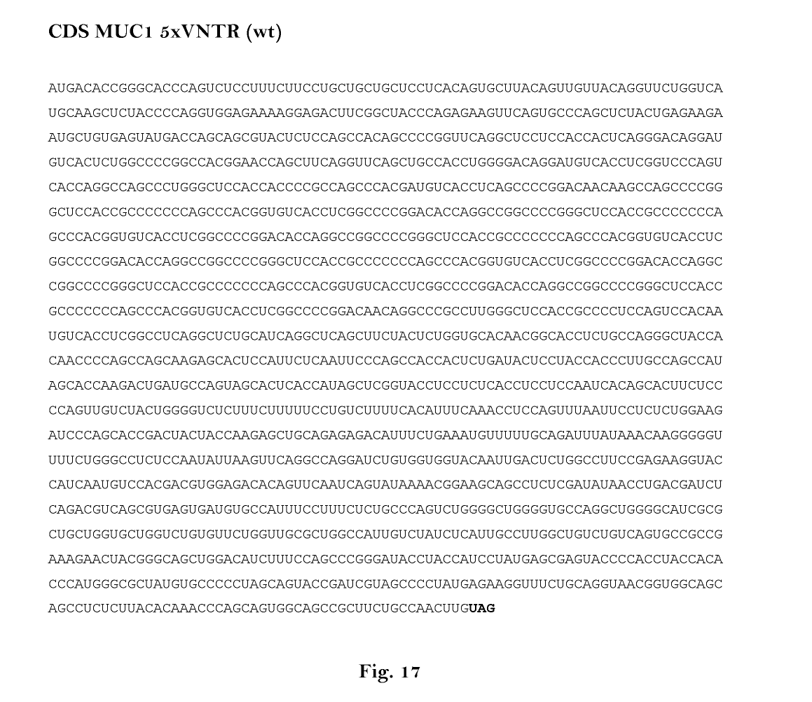

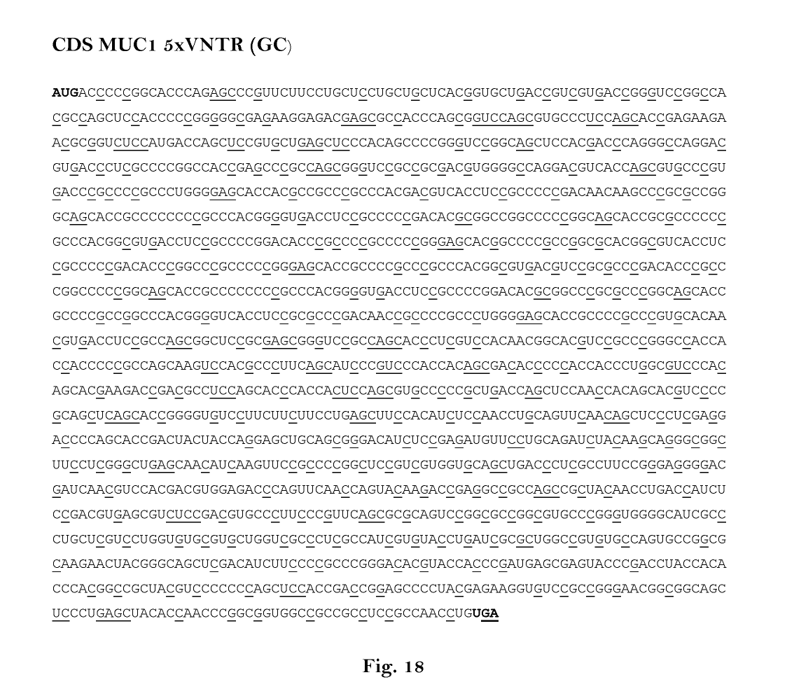

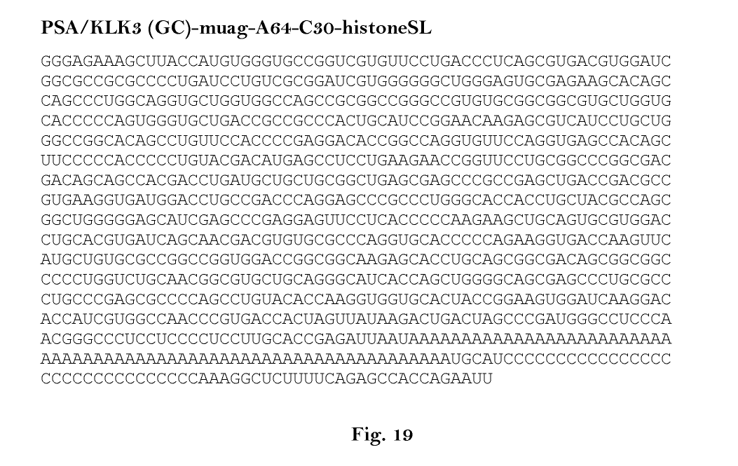

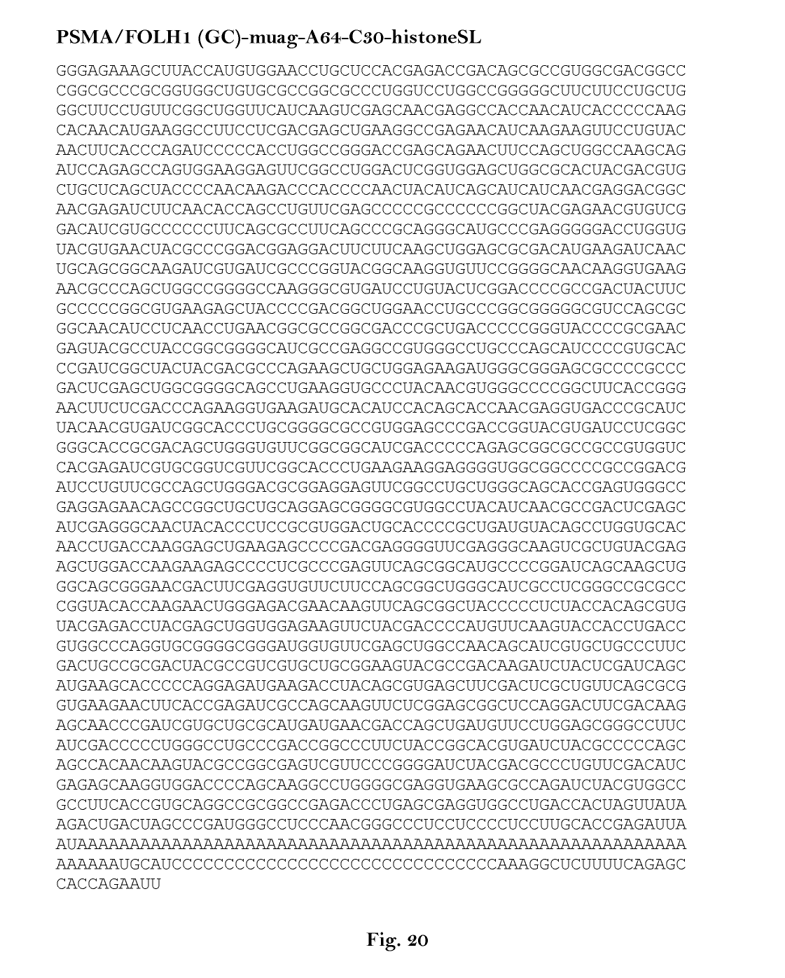

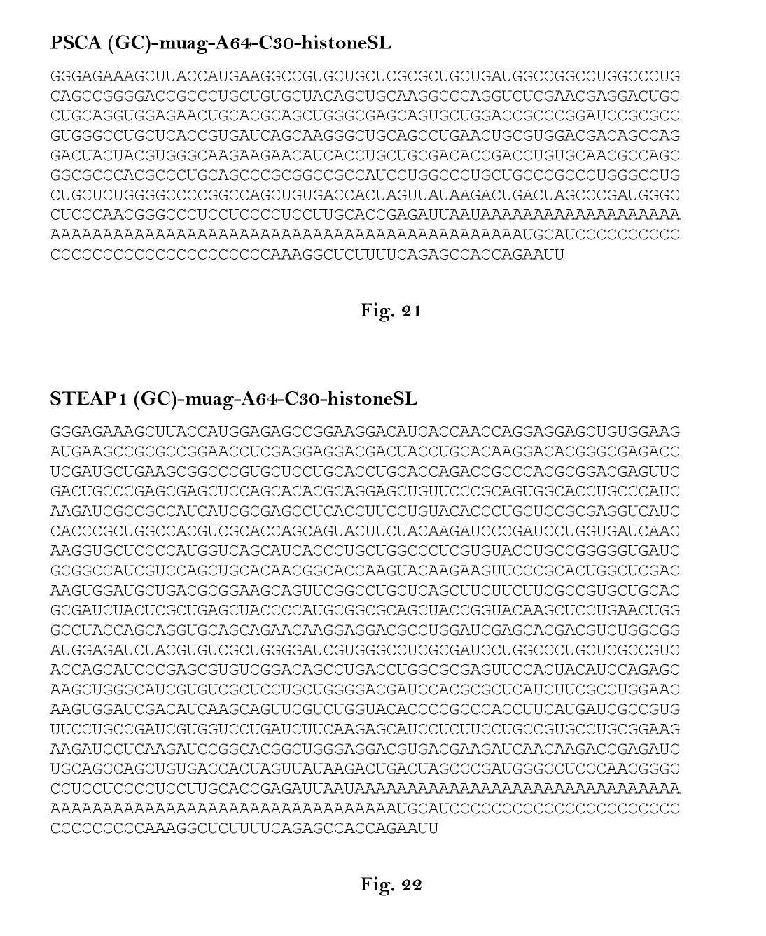

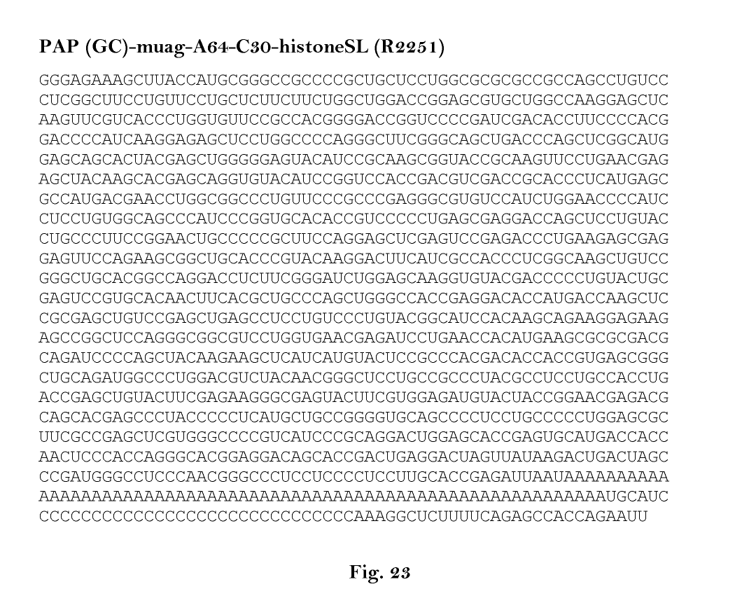

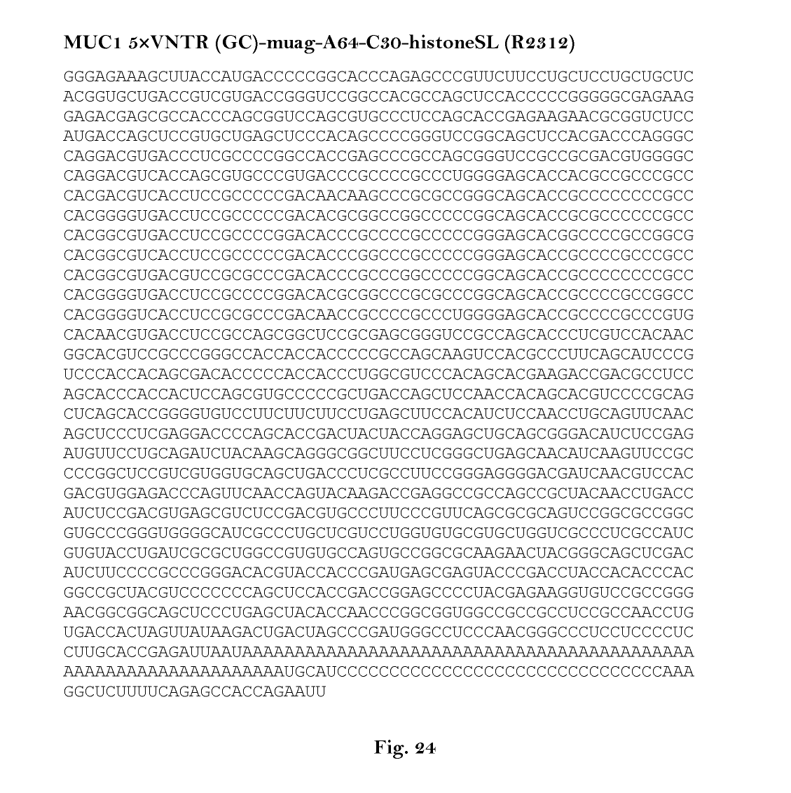

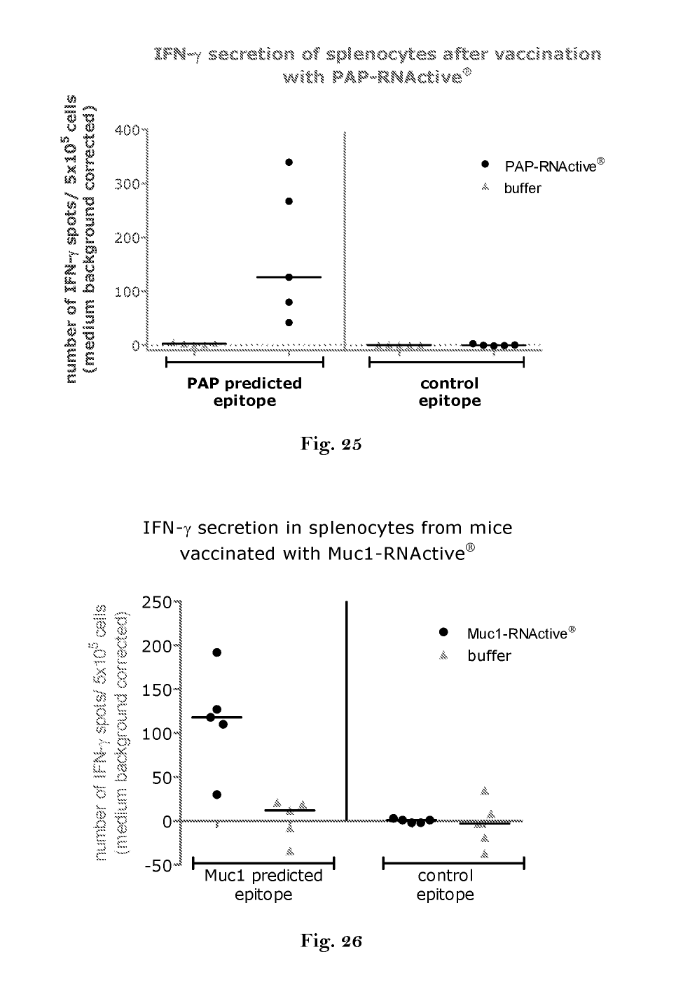

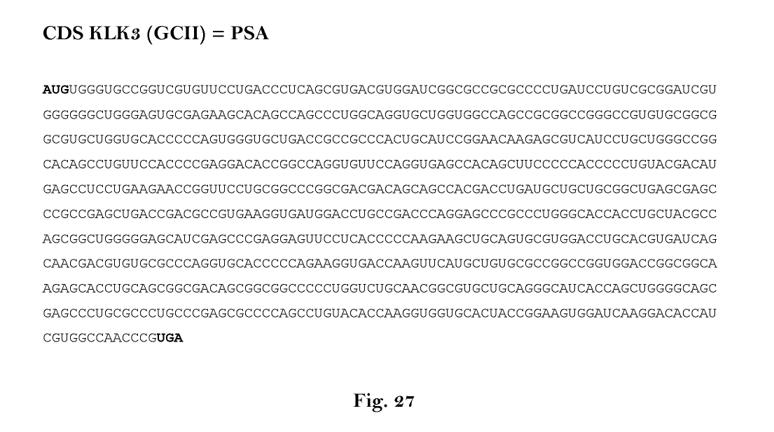

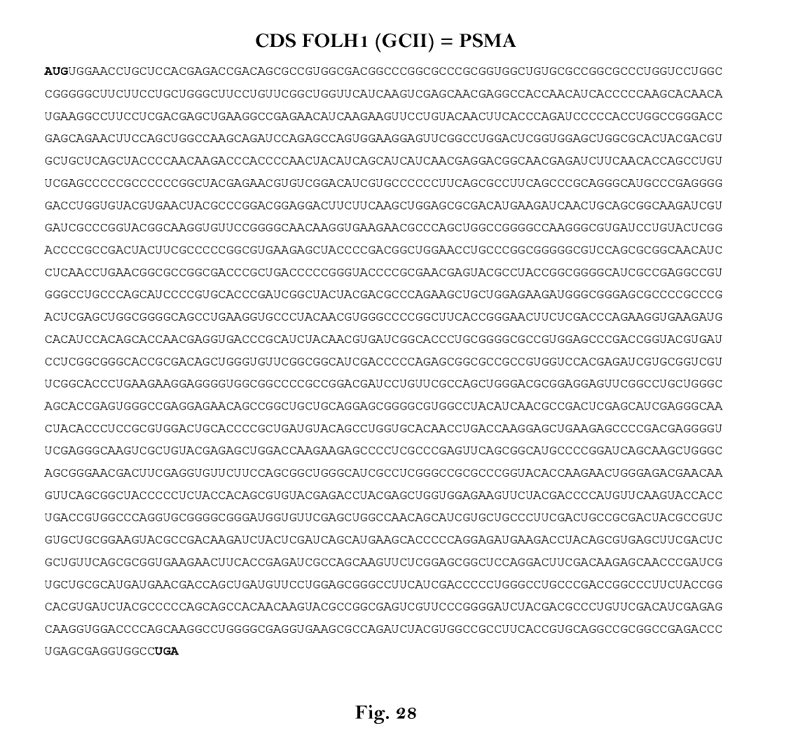

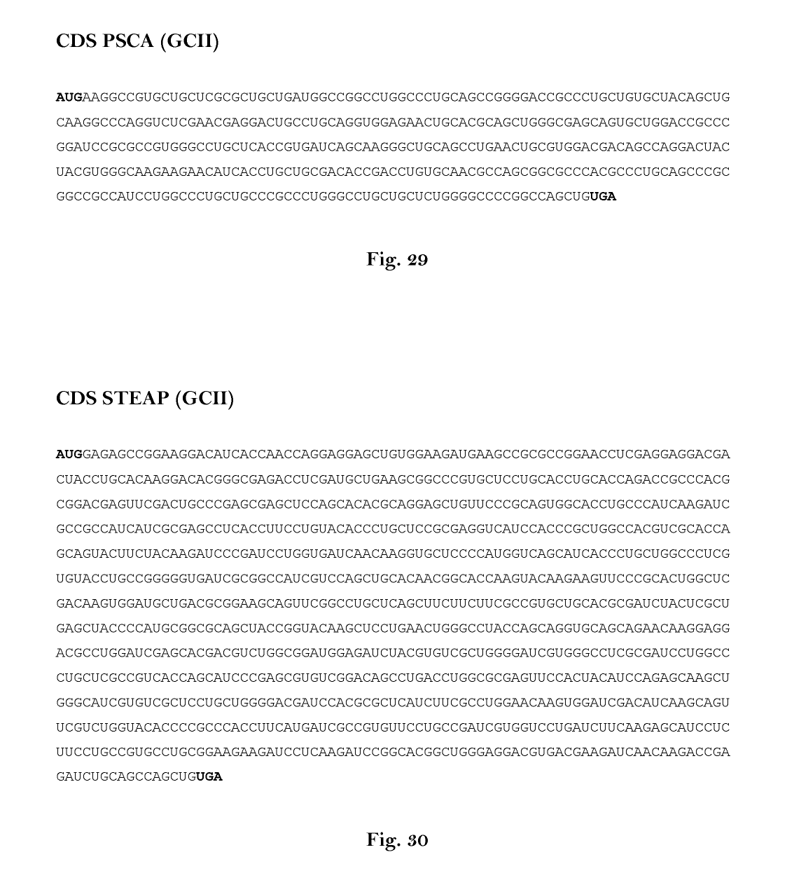

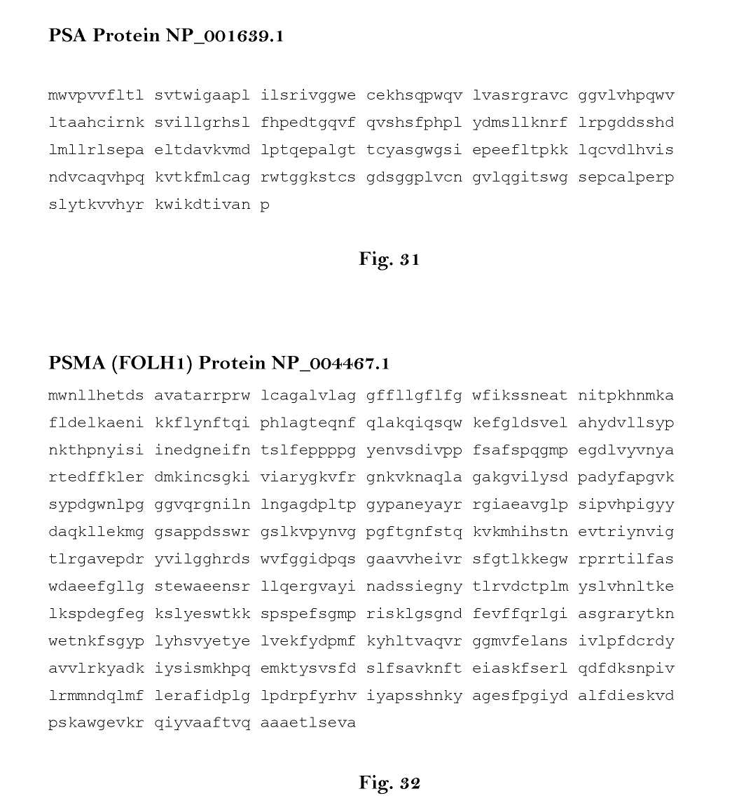

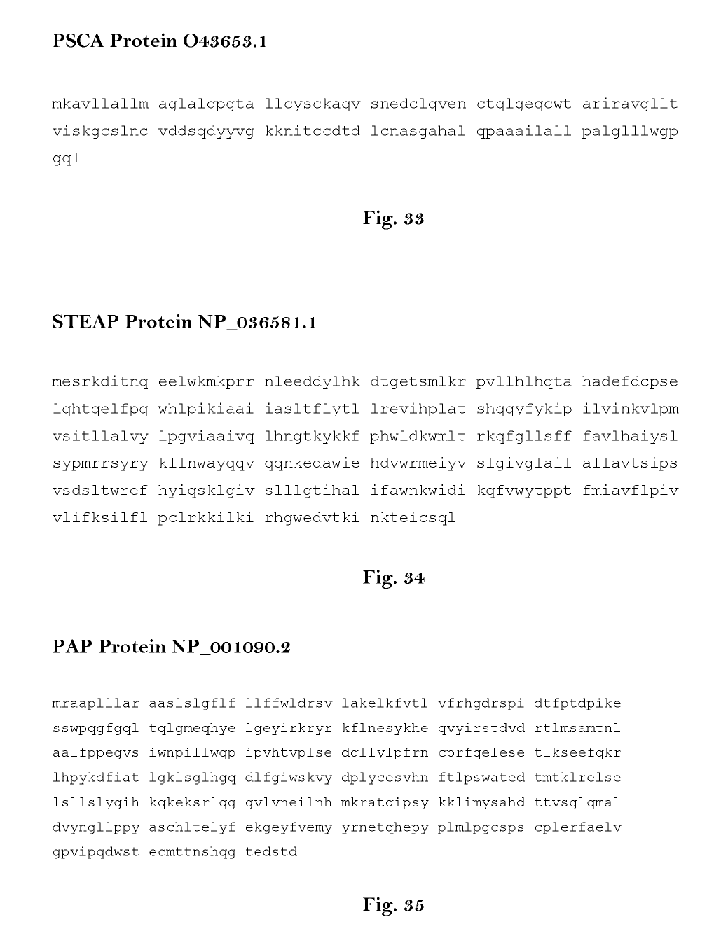

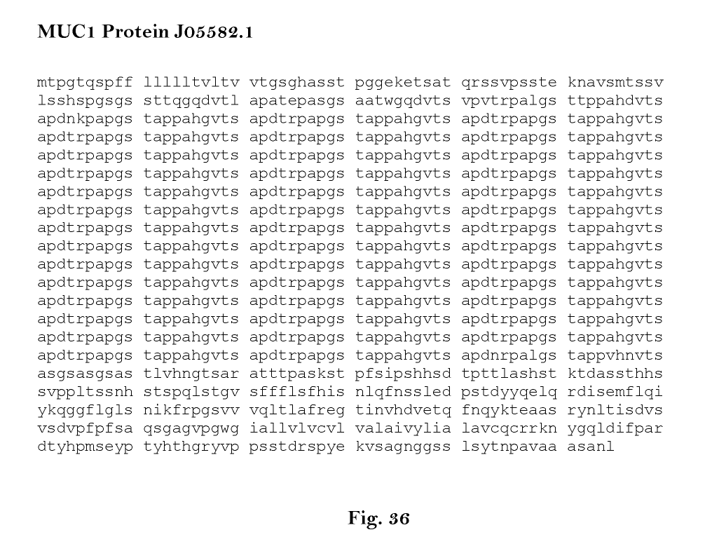

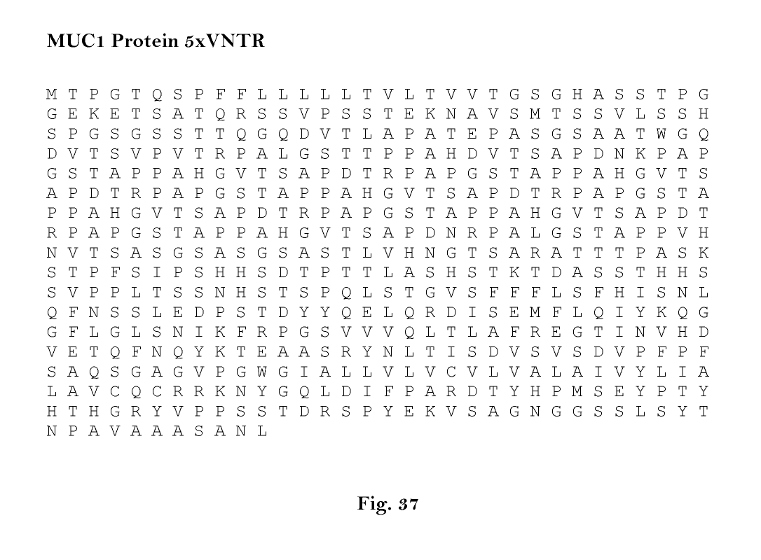

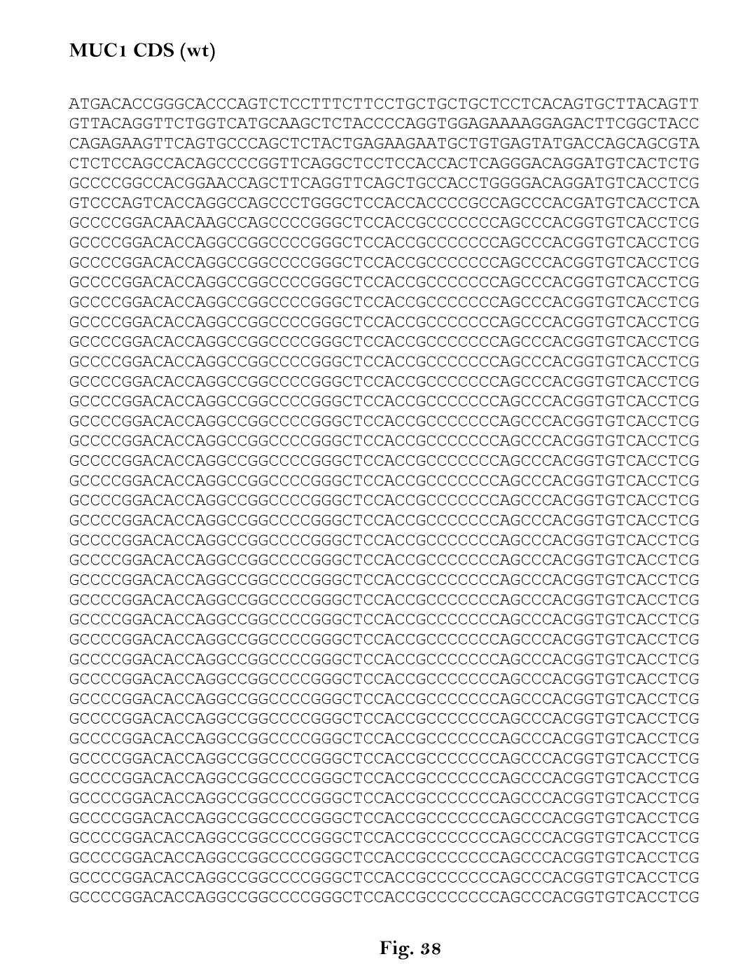

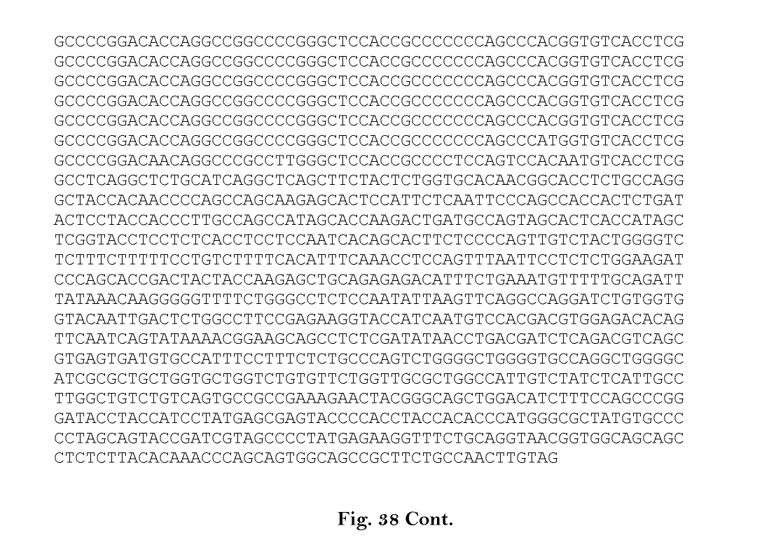

Thereby, the DNA sequence corresponding to the native antigen encoding mRNA (sequences comprising the coding sequences corresponding to the RNA sequences according to FIGS. 2, 5, 8, 11, 14 and 17, i.e. SEQ ID NOs: 2, 5, 8, 11, 14 and 17) were GC-optimized for a better codon-usage obtaining a sequence comprising the coding sequences corresponding to the RNA sequences according to FIGS. 27, 28, 29, 30, 15 and 18, i.e. SEQ ID NOs: 82, 83, 84, 85, 15 and 18. Then, the coding sequence was transferred into a GC-optimized construct (CureVac GmbH, Tubingen, Germany), which has been modified with a poly-A-tail and a poly-C-tail (A64-C30, respectively). The final constructs and the corresponding mRNAs were termed:

KLK3/PSA (GC)-muag-A64-C30 (SEQ ID NO: 1),
FOLH1/PSMA (GC)-muag-A64-C30 (SEQ ID NO: 4),
PSCA (GC)-muag-A64-C30 (SEQ ID NO: 7),
STEAP (GC)-muag-A64-C30 (SEQ ID NO: 10),
PAP (GC)-muag-A64-C30 (SEQ ID NO: 13), and
MUC1-5×VNTR (GC)-muag-A64-C30 (SEQ ID NO: 16), respectively.

The final constructs comprise a sequence corresponding to RNA sequences according to sequences as shown in FIGS. 1, 4, 7, 10, 13 and 16 (SEQ ID NOs: 1, 4, 7, 10, 13 and 16), respectively, which contain following sequence elements: GC-optimized sequence for stabilization and a better codon usage

˜70 Adenosin at the 3′-terminal end (poly-A-tail), 30 Cytosin at the 3′-terminal end (poly-C-tail).

As an alternative the GC-optimized CDS sequences were transferred in GC-optimized constructs, which have been modified with a poly-A-tail, a poly-C-tail and a histone-stem-loop sequence according to SEQ ID NO: 70.

The final constructs and the corresponding RNAs were termed:

KLK3/PSA (GC)-muag-A64-C30-histoneSL (SEQ ID NO: 19),
FOLH1/PSMA (GC)-muag-A64-C30-histoneSL (SEQ ID NO: 20),
PSCA (GC)-muag-A64-C30-histoneSL (SEQ ID NO: 21),
STEAP (GC)-muag-A64-C30-histoneSL (SEQ ID NO: 22),
PAP (GC)-muag-A64-C30-histoneSL (SEQ ID NO: 23), and
MUC1-5×VNTR (GC)-muag-A64-C30-histoneSL (SEQ ID NO: 24), respectively.

The final constructs comprise a sequence corresponding to RNA sequences according to sequences as shown in FIGS. 19, 20, 21, 22, 23 and 24 (SEQ ID NOs: 19, 20, 21, 22, 23 and 24), respectively, which contain following sequence elements:

GC-optimized sequence for stabilization and a better codon usage
˜70 adenosine nucleotides at the 3′-terminal end (poly-A-tail), 30 cytosin nucleotides at the 3′-terminal end (poly-C-tail) and a histone stem-loop sequence according to SEQ ID NO: 71.

### 2. In Vitro Transcription:

Based on the recombinant plasmid DNA obtained in Example 1 the mRNA sequences were prepared by in vitro transcription. Therefore, the recombinant plasmid DNA was linearized and subsequently in vitro transcribed using the T7 RNA polymerase. The DNA template was then degraded by DNase I digestion, and the mRNA was recovered by LiCl precipitation and further cleaned by HPLC extraction (PUREMessenger®, CureVac GmbH, Tubingen, Germany).

3. Complexation with Protamine

Each mRNA encoding an antigen according to the invention was complexed with protamine by addition of protamine to the mRNA in the ratio (1:2) (w/w) (adjuvant component). After incubation for 10 minutes, the same amount of free mRNA used as antigen-providing mRNA was added.

4. Preparation of an mRNA Vaccine and Induction of Antigen-Specific Cytotoxic Antibodies and Antigen-Specific Cytotoxic T-Cells:
4.1 Preparation of an mRNA Vaccine:

The mRNA vaccine consists of GC-optimized mRNAs coding for PAP (SEQ ID NO: 13) or MUC1 (SEQ ID NO: 16), respectively. The mRNA was complexed with protamine by addition of protamine to the mRNA in the ratio (1:2) (w/w) (adjuvant component). After incubation for 10 min, the same amount of free mRNA used as antigen-providing mRNA was added.

The resulting composition was dissolved in 80% (v/v) Ringer-lactate solution.

### 4.2 Vaccination

C57BL/6 mice were vaccinated intradermally with 32 μg of one of the mRNA vaccines as described under 4.1 above. Control mice were treated injected intradermally with buffer (Ringer-lactate). Vaccination comprised five immunizations with 2 immunizations per week. The immune response was analysed 5 or 6 days after completion of the vaccination cycle.

### 4.3 ELISPOT—Detection of CTL (Cytotoxic T Cell) Responses

For the detection of CTL (cytotoxic T cell) responses the analysis of IFN-gamma secretion in response to a specific stimulus can be visualized at a single cell level using the ELISPOT technique.

Splenocytes from mice vaccinated with the mRNA vaccine as described under 4.1 above and control mice were isolated 5 or 6 days after the last vaccination and then transferred into 96-well ELISPOT plates coated with an IFN-gamma capture antibody. The cells were then stimulated for 24 hours at 37° C. using the following peptides:

| | Connexin-derived peptide |
|---|---|
| MUC1-derived peptide | (control) |
| SAPDNRPAL | FEQNTAQP |
| (SEQ ID NO: 72) | (SEQ ID NO: 73) |
| | PSMA-derived peptide |
| PAP-derived peptide | (control) |
| VSIWNPILL | SAVKNFTEI |
| (SEQ ID NO: 74) | (SEQ ID NO: 75) |

After the incubation period the cells were washed out of the plate and the IFN-gamma secreted by the cells was detected using a biotinylated secondary antibody against murine IFN-gamma, followed by streptavidin-AKP. Spots were visualized using BCIP/NBT substrate and counted using an automated ELISPOT reader (Immunospot Analyzer, CTL Analyzers LLC).

### Results:

Intradermal vaccination with both MUC1 and PAP-encoding mRNA constructs led to the activation of antigen-specific CD8⁺ T-cells as demonstrated by the secretion of IFN-gamma in the ELISpot.

### 5. Clinical Trials:

### 5.1. Preparation of the Inventive Vaccine

The mRNA vaccine according to the invention consists of 6 individual, separately formulated GC-optimized mRNAs coding for KLK3/PSA (SEQ ID NO: 19), FOLH1/PSMA (SEQ ID NO: 20), PSCA (SEQ ID NO: 21), STEAP (SEQ ID NO: 22), PAP (SEQ ID NO: 23) or MUC1 (SEQ ID NO: 24), respectively. Each mRNA was complexed with protamine by addition of protamine to the mRNA in the ratio (1:2) (w/w) (adjuvant component). After incubation for 10 min, the same amount of free mRNA used as antigen-providing mRNA was added. After complexation each formulated mRNA was separately lyophilized. For reconstitution, the mRNAs were dissolved in Ringer-Lactate. Each of the six components of the inventive vaccine comprises a formulated and lyophilized mRNA coding for one of the six antigens according to the invention.

### Components of the Inventive Vaccine:

Component 1: 160 μg mRNA coding for KLK3/PSA (SEQ ID NO: 19) complexed with protamine in the ratio (1:2) (w/w) (adjuvant component) and 160 μg mRNA coding for KLK3/PSA (SEQ ID NO: 19)

Component 2: 160 μg mRNA coding for FOLH1/PSMA (SEQ ID NO: 20) complexed with protamine in the ratio (1:2) (w/w) (adjuvant component) and 160 μg mRNA coding for FOLH1/PSMA (SEQ ID NO: 20)

Component 3. 160 μg mRNA coding for PSCA (SEQ ID NO: 21) complexed with protamine in the ratio (1:2) (w/w) (adjuvant component) and 160 μg mRNA coding for PSCA (SEQ ID NO: 21)

Component 4: 160 μg mRNA coding for STEAP (SEQ ID NO: 22) complexed with protamine in the ratio (1:2) (w/w) (adjuvant component) and 160 μg mRNA coding for STEAP (SEQ ID NO: 22),

Component 5: 160 μg mRNA coding for PAP (SEQ ID NO: 23) complexed with protamine in the ratio (1:2) (w/w) (adjuvant component) and 160 μg mRNA coding for PAP (SEQ ID NO: 23)

Component 6: 160 μs mRNA coding for MUC1 (SEQ ID NO: 24) complexed with protamine in the ratio (1:2) (w/w) (adjuvant component) and 160 μg mRNA coding for MUC1 (SEQ ID NO: 24)

Each component is lyophilized and reconstituted for injection with Ringer-Lactate.

### 5.2. Phase II Clinical Trial (Supportive Study):

An open-label, randomized phase II trial including the optional use of an injection device (jet injection) is conducted in patients with intermediate or high risk prostate cancer who receive 4 vaccinations with the inventive mRNA vaccine prepared according to Example 5.1. within a 6-week period prior to radical prostatectomy (neoadjuvant treatment) or are observed without vaccination prior to surgery. Patients treated via conventional intradermal injection receive 320 μg of each mRNA according to Example 5.1. Each component of the inventive vaccine was injected in 2 separate injection sites (160 μg mRNA/injection site). Patients injected by jet injection received only half of the dose (wherein the dose corresponds to 160 μg/component of the inventive vaccine), also injected in 2 separate injection sites (80 μg mRNA/injection site). Patients receive the vaccinations in weeks 1, 2, 3 and 5. These patients undergo radical prostatectomy at least 1 week, but not later than 2 weeks after the 4th vaccination (week 6 or 7). After prostatectomy the patients optionally receive 2 further vaccinations with the inventive vaccine.

### 5.3. Phase IIb Clinical Trial:

A Phase I/II randomised double-blind placebo controlled multicentre study with an open label safety lead-in is conducted in patients with metastatic castration refractory prostate cancer. Patients with asymptomatic or minimally symptomatic disease progressing after surgical castration or gonadotropin-releasing hormone (GNRH) agonist or antagonist therapy and after at least one second-line antihormonal manipulation receive the inventive vaccine prepared according to Example 5.1. or placebo in a 2:1 ratio in favour of the inventive vaccine arm. Treatment with the inventive vaccine is administered on Day 1 of weeks 1, 2, 3, 5, 7, 9, 12, 15, 18 and 24, then every 6 weeks for up to 12 months following the first vaccination and then every 3 months.

A safety lead-in was conducted in 6 patients.

### Treatment Administration:

At every vaccination time point, each of the 6 vaccine components is administered individually on the same day as 2 intradermal (i.d.) injections of 200 μL each per component for a total of 12 injections.

### Results:

As result of the safety lead-in proportion of the study it could be shown that the inventive vaccine is immunogenic and induces a cellular and/or humoral immune response in 83% of the patients.

1. Composition comprising at least one mRNA, wherein the at least one mRNA encodes the following antigens:
STEAP (Six Transmembrane Epithelial Antigen of the Prostate);
PSA (Prostate-Specific Antigen),
PSMA (Prostate-Specific Membrane Antigen),
PSCA (Prostate Stem Cell Antigen);
PAP (Prostatic Acid Phosphatase), and
MUC1 (Mucin 1),
or fragments thereof and wherein the at least one mRNA is mono-, bi- or multicistronic.

2. The composition according to claim 1, wherein each of the antigens or fragments thereof is encoded by a separate mRNA.

3. The composition according to claim 1, wherein STEAP, PSA, PSMA, PSCA, PAP and MUC1 or fragments thereof are encoded by one mRNA.

4. The composition according to claim 1, wherein the antigens or fragments thereof are encoded by at least one bicistronic and/or multicistronic mRNA.

5. The composition according to any of claims 1 to 4, wherein at least one mRNA, preferably at least two mRNAs, more preferably at least three mRNAs, even more preferably at least four mRNAs, even more preferably at least five mRNAs, or even more preferably at least six mRNAs, each comprise at least one coding sequence selected from RNA sequences being identical or at least 80% identical to the RNA sequence of SEQ ID NOs: 2, 5, 8, 11, 14, 17 or 87.

6. The composition according to any of claims 1 to 5, wherein at least one mRNA comprises a coding sequence, which contains or consists of an RNA sequence that is identical or at least 80% identical to the RNA sequences of SEQ ID NOs: 2, 5, 8, 11, 14, 17 or 87.

7. The composition according to any of claims 1 to 6, wherein the at least one mRNA is a modified mRNA, in particular a stabilized mRNA.

8. The composition according to any of claims 1 to 7, wherein the G/C content of the coding region of the at least one mRNA is increased compared to the G/C content of the coding region of the wild-type mRNA, the coded amino acid sequence of the at least one mRNA preferably not being modified compared to the coded amino acid sequence of the wild-type mRNA.

9. The composition according to any of claims 1 to 8, wherein at least one mRNA comprises a coding sequence, which contains or consists of an RNA sequence that is identical or at least 80% identical to the RNA sequences of SEQ ID NOs: 3, 6, 9, 12, 15, 18, 82, 83, 84, or 85.

10. The composition according to any of claims 1 to 9, wherein the at least one mRNA contains a 5′ cap structure and/or the 3′ UTR contains a poly(A) tail, preferably of 10 to 200, 10 to 100, 40 to 80 or 50 to 70 adenosine nucleotides, and/or the 3′ UTR contains a poly(C) tail, preferably of 10 to 200, 10 to 100, 20 to 70, 20 to 60 or 10 to 40 cytosine nucleotides.

11. The composition according to any of claims 1 to 10, wherein the at least one mRNA comprises a 3′ UTR, which comprises (in 5′ to 3′ direction) the following elements:
a) the 3′-UTR derived from the center, α-complex-binding portion of the 3′UTR of an α-globin gene, such as of a human α-globin gene, preferably according to SEQ ID NO: 69:
b) a poly(A) tail, preferably consisting of 10 to 200, 10 to 100, 40 to 80 or 50 to 70 adenosine nucleotides, and
c) a poly(C) tail, preferably consisting of 10 to 200, 10 to 100, 20 to 70, 20 to 60 or 10 to 40 cytosine nucleotides.

12. The composition according to any of claims 1 to 11 comprising six mRNAs, wherein each mRNA encodes a different antigen selected from the group consisting of STEAP (Six Transmembrane Epithelial Antigen of the Prostate), PSA (Prostate-Specific Antigen), PSMA (Prostate-Specific Membrane Antigen), PSCA (Prostate Stem Cell Antigen), PAP (Prostatic Acid Phosphatase) and MUC1 (Mucin 1) and wherein preferably each mRNA comprises an RNA sequence, which is identical or at least 80% identical to an RNA sequence selected from the RNA sequences according to SEQ ID NOs: 1, 4, 7, 10, 13 and 16.

13. The composition according to any of claims 1 to 12, wherein the at least one mRNA comprises a 3′ UTR, which comprises (in 5′ to 3′ direction) the following elements:
a) a poly(A) tail, preferably consisting of 10 to 200, 10 to 100, 40 to 80 or 50 to 70 adenosine nucleotides,
b) a poly(C) tail, preferably consisting of 10 to 200, 10 to 100, 20 to 70, 20 to 60 or 10 to 40 cytosine nucleotides, and
c) a histone stem-loop.

14. The composition according to claim 13, wherein the histone stem-loop is formed by intramolecular base pairing of two neighbouring sequences, which are entirely or partially reverse complementary.

15. The composition according to any of claims 13 to 14, wherein the loop in the histone stem-loop has a length of 3 to 15 bases, preferably of 3 to 10, 3 to 8, 3 to 7, 3 to 6, 4 to 5 or 4 bases.

16. The composition according to any of claims 13 to 15, wherein the sequence forming the stem region in the histone stem-loop has a length of 5 to 10 bases, preferably 5 to 8 bases.

17. The composition according to any of claims 13 to 16, wherein the 3′ UTR of the at least one mRNA contains at least one histone stem-loop that is selected from the following formulae (I) or (II):
formula (I) (stem-loop sequence without stem bordering elements): 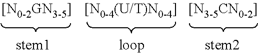
formula (II) (stem-loop sequence with stem bordering elements): 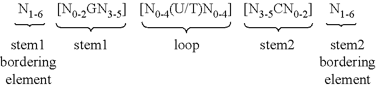
wherein:
stem1 or stem2 bordering elements N1-6 is a consecutive sequence of 1 to 6, preferably of 2 to 6, more preferably of 2 to 5, even more preferably of 3 to 5, most preferably of 4 to 5 or 5 N, wherein each N is independently from another selected from a nucleotide selected from A, U, T, G and C, or a nucleotide analogue thereof;
stem1 [N₀₋₂GN₃₋₅] is reverse complementary or partially reverse complementary with element stem2, and is a consecutive sequence between of 5 to 7 nucleotides;
wherein N₀₋₂ is a consecutive sequence of 0 to 2, preferably of 0 to 1, more preferably of 1 N, wherein each N is independently from another selected from a nucleotide selected from A, U, T, G and C or a nucleotide analogue thereof;
wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof, and
wherein G is guanosine or an analogue thereof, and may be optionally replaced by a cytidine or an analogue thereof, provided that its complementary nucleotide cytidine in stem2 is replaced by guanosine;
loop sequence [N₀₋₄(U/T)N₀₋₄] is located between elements stem1 and stem2, and is a consecutive sequence of 3 to 5 nucleotides, more preferably of 4 nucleotides;
wherein each N₀₋₄ is independent from another a consecutive sequence of 0 to 4, preferably of 1 to 3, more preferably of 1 to 2 N, wherein each N is independently from another selected from a nucleotide selected from A, U, T, G and C or a nucleotide analogue thereof; and
wherein U/T represents uridine, or optionally thymidine;
stem2 [N₃₋₅CN₀₋₂] is reverse complementary or partially reverse complementary with element stem1, and is a consecutive sequence between of 5 to 7 nucleotides;
wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof;
wherein N₀₋₂ is a consecutive sequence of 0 to 2, preferably of 0 to 1, more preferably of 1 N, wherein each N is independently from another selected from a nucleotide selected from A, U, T, G and C or a nucleotide analogue thereof; and
wherein C is cytidine or an analogue thereof, and may be optionally replaced by a guanosine or an analogue thereof provided that its complementary nucleotide guanosine in stem1 is replaced by cytidine;
wherein
stem1 and stem2 are capable of base pairing with each other
forming a reverse complementary sequence, wherein base pairing may occur between stem1 and stem2, or
forming a partially reverse complementary sequence, wherein an incomplete base pairing may occur between stem1 and stem2.

18. The composition according to any of claims 13 to 17, wherein the at least one histone stem-loop is selected from at least one of the following formulae (Ia) or (IIa):
formula (Ia) (stem-loop sequence without stem bordering elements): 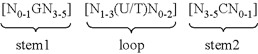
formula (IIa) (stem-loop sequence with stem bordering elements):

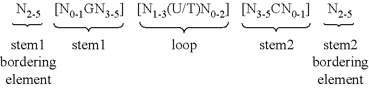

19. The composition according to any of claims 13 to 18 comprising any one of the histone stem loop nucleotide sequences according to SEQ ID NOs: 25 to 66, preferably a nucleotide sequence according to SEQ ID NO. 70 and most preferably a RNA sequence according to SEQ ID NO. 71.

20. The composition according to any of claims 1 to 19, wherein the at least one mRNA comprises at least one mRNA identical or at least 80% identical to an RNA sequence according to any of the RNA sequences according to SEQ ID NOs: 19 to 24.

21. The composition according to any of claims 1 to 20 comprising six mRNAs, wherein each mRNA encodes a different antigen selected from the group consisting of STEAP (Six Transmembrane Epithelial Antigen of the Prostate), PSA (Prostate-Specific Antigen), PSMA (Prostate-Specific Membrane Antigen), PSCA (Prostate Stem Cell Antigen), PAP (Prostatic Acid Phosphatase) and MUC1 (Mucin 1) and each mRNA is identical or at least 80% identical to a RNA sequence selected from the RNA sequences according to SEQ ID NO: 19, 20, 21, 22, 23 or 24.

22. The composition according to any of claims 1 to 21 comprising six mRNAs, wherein one mRNA encodes PSA and is identical or at least 80% identical to SEQ ID NO: 19, one mRNA encodes PSMA and is identical or at least 80% identical to SEQ ID NO: 20, one mRNA encodes PSCA and is identical or at least 80% identical to SEQ ID NO: 21, one mRNA encodes STEAP and is identical or at least 80% identical to SEQ ID NO: 22, one mRNA encodes PAP and is identical or at least 80% identical to SEQ ID NO: 23 and one mRNA encodes MUC1 and is identical or at least 80% identical to SEQ ID NO: 24.

23. The composition according to any of claims 1 to 22, wherein the at least one mRNA is complexed with one or more polycations, preferably with protamine or oligofectamine, most preferably with protamine.

24. The composition according to claim 23, wherein the N/P ratio of the at least one mRNA to the one or more polycations is in the range of about 0.1 to 10, including a range of about 0.3 to 4, of about 0.5 to 2, of about 0.7 to 2 and of about 0.7 to 1.5.

25. The composition according to any of claims 1 to 24 comprising at least one mRNA, which is complexed with one or more polycations, and at least one free mRNA.

26. The composition according to claim 25, wherein the complexed mRNA is identical to the free mRNA.

27. The composition according to claim 25 or 26, wherein the molar ratio of the complexed mRNA to the free mRNA is selected from a molar ratio of about 0.001:1 to about 1:0.001, including a ratio of about 1:1.

28. The composition according to any of claims 1 to 27, wherein the composition additionally comprises at least one adjuvant.

29. The composition according to any of claims 1 to 28, wherein the at least one adjuvant is selected from the group consisting of:
cationic or polycationic compounds, comprising cationic or polycationic peptides or proteins, including protamine, nucleoline, spermin or spermidine, poly-L-lysine (PLL), poly-arginine, basic polypeptides, cell penetrating peptides (CPPs), including HIV-binding peptides, Tat, HIV-1 Tat (HIV), Tat-derived peptides, Penetratin, VP22 derived or analog peptides, HSV VP22 (Herpes simplex), MAP, KALA or protein transduction domains (PTDs, PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, protamine, spermine, spermidine, or histones, cationic polysaccharides, including chitosan, polybrene, cationic polymers, including polyethyleneimine (PEI), cationic lipids, including DOTMA: 1-(2,3-sioleyloxy)propyl)□-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, DOTAP: dioleoyloxy-3-(trimethylammonio)propane, DC-6-14: O,O-ditetradecanoyl-N-(-trimethylammonioacetyl)diethanolamine chloride, CLIP1: rac-(2,3-dioctadecyloxypropyl)(2-hydroxyethyl)-dimethylammonium chloride, CLIP6: rac-2(2,3-dihexadecyloxypropyl-oxymethyloxy)ethyl trimethylammonium, CLIPS: rac-2(2,3-dihexadecyloxypropyl-oxysuccinyloxy)ethyl-trimethylammonium, oligofectamine, or cationic or polycationic polymers, including modified polyaminoacids, including—aminoacid-polymers or reversed polyamides, modified polyethylenes, including PVP (poly(N-ethyl-4-vinylpyridinium bromide)), modified acrylates, including pDMAEMA (poly(dimethylaminoethyl methylacrylate)), modified Amidoamines including pAMAM (poly(amidoamine)), modified polybetaaminoester (PBAE), including diamine end modified 1,4 butanediol diacrylate-co-5-amino-1-pentanol polymers, dendrimers, including polypropylamine dendrimers or pAMAM based dendrimers, polyimine(s), including PEI: poly(ethyleneimine), poly(propyleneimine), polyallylamine, sugar backbone based polymers, including cyclodextrin based polymers, dextran based polymers, Chitosan, etc., silan backbone based polymers, such as PMOXA-PDMS copolymers, etc., Blockpolymers consisting of a combination of one or more cationic blocks selected of a cationic polymer as mentioned before, and of one or more hydrophilic- or hydrophobic blocks (e.g polyethyleneglycole);
or
cationic or polycationic proteins or peptides, selected from following proteins or peptides having the following total formula (III): (Arg)l;(Lys)m;(His)n;(Orn)o;(Xaa)x, wherein l+m+n+o+x=8-15, and l, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and
Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide; or
nucleic acids having the formula (IV): GlXmGn, wherein: G is guanosine, uracil or an analogue of guanosine or uracil; X is guanosine, uracil, adenosine, thymidine, cytosine or an analogue of the above-mentioned nucleotides; l is an integer from 1 to 40, wherein when l=1 G is guanosine or an analogue thereof, when l>1 at least 50% of the nucleotides are guanosine or an analogue thereof; m is an integer and is at least 3; wherein when m=3 X is uracil or an analogue thereof, when m>3 at least 3 successive uracils or analogues of uracil occur; n is an integer from 1 to 40, wherein when n=1 G is guanosine or an analogue thereof, when n>1 at least 50% of the nucleotides are guanosine or an analogue thereof;
or
nucleic acids having the formula (V): ClXmCn, wherein: C is cytosine, uracil or an analogue of cytosine or uracil; X is guanosine, uracil, adenosine, thymidine, cytosine or an analogue of the above-mentioned nucleotides; l is an integer from 1 to 40, wherein when l=1 C is cytosine or an analogue thereof, when l>1 at least 50% of the nucleotides are cytosine or an analogue thereof; m is an integer and is at least 3; wherein when m=3 X is uracil or an analogue thereof, when m>3 at least 3 successive uracils or analogues of uracil occur; n is an integer from 1 to 40, wherein when n=1 C is cytosine or an analogue thereof, when n>1 at least 50% of the nucleotides are cytosine or an analogue thereof.

30. A vaccine, comprising a composition according to any of claims 1 to 29.

31. The vaccine according to claim 30, wherein the composition according to any of claims 1 to 29 elicits an adaptive immune response.

32. The vaccine according to claim 30 or 31, wherein the vaccine further comprises a pharmaceutically acceptable carrier.

33. The vaccine according to any of claims 30 to 32, wherein at least one mRNA of the composition is administered to the subject individually.

34. The composition according to any of claims 1 to 29 for use as a vaccine for the treatment of prostate cancer (PCa), preferably of prostate adenocarcinoma, locally limited, locally advanced, metastatic, castration-resistant (hormone-refractory), metastatic castration-resistant and non-metastatic castration-resistant prostate cancers, and diseases or disorders related thereto.

35. Use of a combination of six mRNAs for the treatment of prostate cancer, wherein each mRNA encodes one antigen selected from the group consisting of STEAP (Six Transmembrane Epithelial Antigen of the Prostate), PSA (Prostate-Specific Antigen), PSMA (Prostate-Specific Membrane Antigen), PSCA (Prostate Stem Cell Antigen), PAP (Prostatic Acid Phosphatase) and MUC1 (Mucin 1).

36. Use according to claim 35, wherein
one mRNA comprises a coding sequence, which encodes PSA and contains or consists of an RNA sequence that is identical or at least 80% identical to the RNA sequences of SEQ ID NOs: 2, 3 or 82;
one mRNA comprises a coding sequence, which encodes PSMA and contains or consists of an RNA sequence that is identical or at least 80% identical to the RNA sequences of SEQ ID NOs: 5, 6 or 83;
one mRNA comprises a coding sequence, which encodes PSCA and contains or consists of an RNA sequence that is identical or at least 80% identical to the RNA sequences of SEQ ID NOs: 8, 9 or 84;
one mRNA comprises a coding sequence, which encodes STEAP and contains or consists of an RNA sequence that is identical or at least 80% identical to the RNA sequences of SEQ ID NOs: 11, 12 or 85;
one mRNA comprises a coding sequence, which encodes PAP and contains or consists of an RNA sequence that is identical or at least 80% identical to the RNA sequences of SEQ ID NOs: 14 or 15;
one mRNA comprises a coding sequence, which encodes MUC1 and contains or consists of an RNA sequence that is identical or at least 80% identical to the RNA sequences of SEQ ID NOs: 17, 18 or 87.

37. Use according to any of claim 35 or 36, wherein at least one mRNA comprises a histone stem-loop in the 3′ UTR region.

38. Use according to any of claims 35 to 37, wherein each mRNA comprises an RNA sequence that is identical or at least 80% identical to a different one of the RNA sequences according to SEQ ID NOs: 19 to 24.

39. Use according to any of claims 35 to 38 comprising six mRNAs, wherein one mRNA encodes PSA and is identical or at least 80% identical to SEQ ID NO: 19, one mRNA encodes PSMA and is identical or at least 80% identical to SEQ ID NO: 20, one mRNA encodes PSCA and is identical or at least 80% identical to SEQ ID NO: 21, one mRNA encodes STEAP and is identical or at least 80% identical to SEQ ID NO: 22, one mRNA encodes PAP and is identical or at least 80% identical to SEQ ID NO: 23 and one mRNA encodes MUC1 and is identical or at least 80% identical to SEQ ID NO: 24

40. Use according to any of claims 35 to 39, wherein each of the six mRNAs is administered separately.

41. Use according to any of claims 35 to 40, wherein the mRNAs are administered by intradermal injection.

42. Use according to any of claims 35 to 41, wherein the treatment is assisted by co-therapy, e.g. prostate surgery, radiotherapy, hormone therapy and/or chemotherapy.

43. A kit, preferably kit of parts, comprising the composition according to any of claims 1 to 29, and/or a vaccine according to any of claims 30 to 33, and optionally a liquid vehicle for solubilising and optionally technical instructions with information on the administration and dosage of the active composition and/or the vaccine.

44. The kit according to claim 43, wherein the kit is a kit of parts and each part of the kit contains at least one mRNA preferably encoding a different antigen selected from the antigens defined in claim 1, all parts of the kit of parts forming the composition or the vaccine of the preceding claims.

45. The kit according to claim 43 or 44, wherein the kit contains at least two parts containing six mRNAs.

46. The kit according to any of claims 43 to 45, wherein all six mRNAs are provided in lyophilized form in separate parts.

47. The kit according to any of claims 43 to 46, wherein the kit contains as a part Ringer-Lactate solution.

48. The kit according to any of claims 43 to 47, wherein the kit contains six parts, each part containing one of the six mRNAs.


SEQUENCE LISTING (87 sequences)

SEQ ID NO: 1 - RNA, 990 nt, Artificial Sequence
  CAP-KLK3(GC)-muag-A64-C30
gggagaaagcuuaccaugugggugccggucguguuccugacccucagcgugacguggaucggcgccgcgccccugauccugucgcggaucguggggggcugggagugcgagaagcacagccagcccuggcaggugcugguggccagccgcggccgggccgugugcggcggcgugcuggugcacccccagugggugcugaccgccgcccacugcauccggaacaagagcgucauccugcugggccggcacagccuguuccaccccgaggacaccggccagguguuccaggugagccacagcuucccccacccccuguacgacaugagccuccugaagaaccgguuccugcggcccggcgacgacagcagccacgaccugaugcugcugcggcugagcgagcccgccgagcugaccgacgccgugaaggugauggaccugccgacccaggagcccgcccugggcaccaccugcuacgccagcggcugggggagcaucgagcccgaggaguuccucacccccaagaagcugcagugcguggaccugcacgugaucagcaacgacgugugcgcccaggugcacccccagaaggugaccaaguucaugcugugcgccggccgguggaccggcggcaagagcaccugcagcggcgacagcggcggcccccuggucugcaacggcgugcugcagggcaucaccagcuggggcagcgagcccugcgcccugcccgagcgccccagccuguacaccaagguggugcacuaccggaaguggaucaaggacaccaucguggccaacccgugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuagacaauuggaauu

SEQ ID NO: 2 - RNA, 786 nt, Artificial Sequence
  CDS KLK3(wt)
augugggucccgguugucuuccucacccuguccgugacguggauuggugcugcaccccucauccugucucggauugugggaggcugggagugcgagaagcauucccaacccuggcaggugcuuguggccucucguggcagggcagucugcggcgguguucuggugcacccccaguggguccucacagcugcccacugcaucaggaacaaaagcgugaucuugcugggucggcacagccuguuucauccugaagacacaggccagguauuucaggucagccacagcuucccacacccgcucuacgauaugagccuccugaagaaucgauuccucaggccaggugaugacuccagccacgaccucaugcugcuccgccugucagagccugccgagcucacggaugcugugaaggucauggaccugcccacccaggagccagcacuggggaccaccugcuacgccucaggcuggggcagcauugaaccagaggaguucuugaccccaaagaaacuucaguguguggaccuccauguuauuuccaaugacgugugugcgcaaguucacccucagaaggugaccaaguucaugcugugugcuggacgcuggacagggggcaaaagcaccugcucgggugauucugggggcccacuugucuguaauggugugcuucaagguaucacgucauggggcagugaaccaugugcccugcccgaaaggccuucccuguacaccaagguggugcauuaccggaaguggaucaaggacaccaucguggccaaccccuga

SEQ ID NO: 3 - RNA, 786 nt, Artificial Sequence
  CDS KLK3(GC)
augugggugcccgucguguuccugacccucagcgugaccuggaucggcgccgccccgcugauccugucccggaucgucgggggcugggagugcgagaagcacagccagcccuggcaggugcucguggcgucccgcgggcgggccgucugcggcggggugcuggugcacccccaguggguccugacggccgcccacugcauccgcaacaagagcgugauccuccugggccggcacucccuguuccaccccgaggacaccggccagguguuccaggucagccacuccuucccgcacccccucuacgacaugagccugcugaagaaccgcuuccuccggcccggggacgacuccagccacgaccugaugcugcuccgccuguccgagcccgccgagcugaccgacgcggugaaggugauggaccucccgacccaggagcccgcccugggcacgaccugcuacgccagcggguggggcuccaucgagcccgaggaguuccugacccccaagaagcuccagugcgucgaccugcacgugaucagcaacgacgugugcgcccagguccacccgcagaaggugaccaaguucaugcugugcgcggggcgguggacgggcggcaaguccaccugcagcggggacuccggcgggccccucgugugcaacggcguccugcagggcaucaccagcuggggguccgagcccugcgcccugcccgagcgcccgagccucuacaccaagguggugcacuaccggaaguggaucaaggacacgaucgucgccaaccccuga

SEQ ID NO: 4 - RNA, 2457 nt, Artificial Sequence
  CAP-FOLH1(GC)-muag-A64-C30
gggagaaagcuuaccauguggaaccugcuccacgagaccgacagcgccguggcgacggcccggcgcccgcgguggcugugcgccggcgcccugguccuggccgggggcuucuuccugcugggcuuccuguucggcugguucaucaagucgagcaacgaggccaccaacaucacccccaagcacaacaugaaggccuuccucgacgagcugaaggccgagaacaucaagaaguuccuguacaacuucacccagaucccccaccuggccgggaccgagcagaacuuccagcuggccaagcagauccagagccaguggaaggaguucggccuggacucgguggagcuggcgcacuacgacgugcugcucagcuaccccaacaagacccaccccaacuacaucagcaucaucaacgaggacggcaacgagaucuucaacaccagccuguucgagcccccgccccccggcuacgagaacgugucggacaucgugccccccuucagcgccuucagcccgcagggcaugcccgagggggaccugguguacgugaacuacgcccggacggaggacuucuucaagcuggagcgcgacaugaagaucaacugcagcggcaagaucgugaucgcccgguacggcaagguguuccggggcaacaaggugaagaacgcccagcuggccggggccaagggcgugauccuguacucggaccccgccgacuacuucgcccccggcgugaagagcuaccccgacggcuggaaccugcccggcgggggcguccagcgcggcaacauccucaaccugaacggcgccggcgacccgcugacccccggguaccccgcgaacgaguacgccuaccggcggggcaucgccgaggccgugggccugcccagcauccccgugcacccgaucggcuacuacgacgcccagaagcugcuggagaagaugggcgggagcgccccgcccgacucgagcuggcggggcagccugaaggugcccuacaacgugggccccggcuucaccgggaacuucucgacccagaaggugaagaugcacauccacagcaccaacgaggugacccgcaucuacaacgugaucggcacccugcggggcgccguggagcccgaccgguacgugauccucggcgggcaccgcgacagcuggguguucggcggcaucgacccccagagcggcgccgccgugguccacgagaucgugcggucguucggcacccugaagaaggagggguggcggccccgccggacgauccuguucgccagcugggacgcggaggaguucggccugcugggcagcaccgagugggccgaggagaacagccggcugcugcaggagcggggcguggccuacaucaacgccgacucgagcaucgagggcaacuacacccuccgcguggacugcaccccgcugauguacagccuggugcacaaccugaccaaggagcugaagagccccgacgagggguucgagggcaagucgcuguacgagagcuggaccaagaagagccccucgcccgaguucagcggcaugccccggaucagcaagcugggcagcgggaacgacuucgagguguucuuccagcggcugggcaucgccucgggccgcgcccgguacaccaagaacugggagacgaacaaguucagcggcuacccccucuaccacagcguguacgagaccuacgagcugguggagaaguucuacgaccccauguucaaguaccaccugaccguggcccaggugcggggcgggaugguguucgagcuggccaacagcaucgugcugcccuucgacugccgcgacuacgccgucgugcugcggaaguacgccgacaagaucuacucgaucagcaugaagcacccccaggagaugaagaccuacagcgugagcuucgacucgcuguucagcgcggugaagaacuucaccgagaucgccagcaaguucucggagcggcuccaggacuucgacaagagcaacccgaucgugcugcgcaugaugaacgaccagcugauguuccuggagcgggccuucaucgacccccugggccugcccgaccggcccuucuaccggcacgugaucuacgcccccagcagccacaacaaguacgccggcgagucguucccggggaucuacgacgcccuguucgacaucgagagcaagguggaccccagcaaggccuggggcgaggugaagcgccagaucuacguggccgccuucaccgugcaggccgcggccgagacccugagcgagguggccugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuagacaauuggaauu

SEQ ID NO: 5 - RNA, 2253 nt, Artificial Sequence
  CDS FOLH1(wt)
auguggaaucuccuucacgaaaccgacucggcuguggccaccgcgcgccgcccgcgcuggcugugcgcuggggcgcuggugcuggcggguggcuucuuucuccucggcuuccucuucgggugguuuauaaaauccuccaaugaagcuacuaacauuacuccaaagcauaauaugaaagcauuuuuggaugaauugaaagcugagaacaucaagaaguucuuauauaauuuuacacagauaccacauuuagcaggaacagaacaaaacuuucagcuugcaaagcaaauucaaucccaguggaaagaauuuggccuggauucuguugagcuagcacauuaugauguccuguuguccuacccaaauaagacucaucccaacuacaucucaauaauuaaugaagauggaaaugagauuuucaacacaucauuauuugaaccaccuccuccaggauaugaaaauguuucggauauuguaccaccuuucagugcuuucucuccucaaggaaugccagagggcgaucuaguguauguuaacuaugcacgaacugaagacuucuuuaaauuggaacgggacaugaaaaucaauugcucugggaaaauuguaauugccagauaugggaaaguuuucagaggaaauaagguuaaaaaugcccagcuggcaggggccaaaggagucauucucuacuccgacccugcugacuacuuugcuccuggggugaaguccuauccagaugguuggaaucuuccuggaggugguguccagcguggaaauauccuaaaucugaauggugcaggagacccucucacaccagguuacccagcaaaugaauaugcuuauaggcguggaauugcagaggcuguuggucuuccaaguauuccuguucauccaauuggauacuaugaugcacagaagcuccuagaaaaaauggguggcucagcaccaccagauagcagcuggagaggaagucucaaagugcccuacaauguuggaccuggcuuuacuggaaacuuuucuacacaaaaagucaagaugcacauccacucuaccaaugaagugacaagaauuuacaaugugauagguacucucagaggagcaguggaaccagacagauaugucauucugggaggucaccgggacucauggguguuuggugguauugacccucagaguggagcagcuguuguucaugaaauugugaggagcuuuggaacacugaaaaaggaaggguggagaccuagaagaacaauuuuguuugcaagcugggaugcagaagaauuuggucuucuugguucuacugagugggcagaggagaauucaagacuccuucaagagcguggcguggcuuauauuaaugcugacucaucuauagaaggaaacuacacucugagaguugauuguacaccgcugauguacagcuugguacacaaccuaacaaaagagcugaaaagcccugaugaaggcuuugaaggcaaaucucuuuaugaaaguuggacuaaaaaaaguccuuccccagaguucaguggcaugcccaggauaagcaaauugggaucuggaaaugauuuugagguguucuuccaacgacuuggaauugcuucaggcagagcacgguauacuaaaaauugggaaacaaacaaauucagcggcuauccacuguaucacagugucuaugaaacauaugaguugguggaaaaguuuuaugauccaauguuuaaauaucaccucacuguggcccagguucgaggagggaugguguuugagcuagccaauuccauagugcucccuuuugauugucgagauuaugcuguaguuuuaagaaaguaugcugacaaaaucuacaguauuucuaugaaacauccacaggaaaugaagacauacaguguaucauuugauucacuuuuuucugcaguaaagaauuuuacagaaauugcuuccaaguucagugagagacuccaggacuuugacaaaagcaacccaauaguauuaagaaugaugaaugaucaacucauguuucuggaaagagcauuuauugauccauuaggguuaccagacaggccuuuuuauaggcaugucaucuaugcuccaagcagccacaacaaguaugcaggggagucauucccaggaauuuaugaugcucuguuugauauugaaagcaaaguggacccuuccaaggccuggggagaagugaagagacagauuuauguugcagccuucacagugcaggcagcugcagagacuuugagugaaguagccuaa

SEQ ID NO: 6 - RNA, 2253 nt, Artificial Sequence
  CDS FOLH1(GC)
auguggaaccugcuccacgagaccgacagcgccguggccaccgcgcggcgcccccgguggcugugcgccggcgcccugguccucgccgggggcuucuuccugcugggguuccucuucggcugguucaucaaguccagcaacgaggccacgaacaucaccccgaagcacaacaugaaggcguuccuggacgagcugaaggccgagaacaucaagaaguuccucuacaacuucacccagaucccccaccuggccgggaccgagcagaacuuccagcuggccaagcagauccagucccaguggaaggaguucggccucgacagcguggagcuggcgcacuacgacgugcugcucuccuaccccaacaagacgcaccccaacuacaucagcaucaucaacgaggacggcaacgagaucuucaacaccucccuguucgagccgcccccccccggguacgagaacgucagcgacaucgugccgcccuucuccgccuucagcccccagggcaugcccgagggggaccugguguacgucaacuacgcccgcaccgaggacuucuucaagcucgagcgggacaugaagaucaacugcuccggcaagaucgugaucgcccgcuacgggaagguguuccggggcaacaaggucaagaacgcccagcuggcgggcgccaagggggugauccuguacagcgacccggccgacuacuucgcccccggcgugaaguccuaccccgacggguggaaccuccccggcggcgggguccagcgcggcaacauccugaaccugaacggggccggcgacccgcucacccccggguaccccgcgaacgaguacgccuaccggcgcggcaucgccgaggccgugggccugcccagcaucccggugcaccccaucggguacuacgacgcccagaagcugcucgagaagaugggcggguccgcgccccccgacagcuccuggcggggcagccugaaggucccguacaacguggggcccggcuucacgggcaacuucuccacccagaaggugaagaugcacauccacagcaccaacgaggucacccgcaucuacaacgugaucgggacgcugcggggcgccguggagcccgaccgcuacgucauccucgggggccaccgggacagcuggguguucgggggcaucgacccccaguccggcgccgccgugguccacgagaucgugcgcagcuucgggacccugaagaaggagggcuggcggccgcgccggaccauccuguucgccuccugggacgcggaggaguucgggcuccugggcagcaccgagugggccgaggagaacucccgccugcuccaggagcggggcgucgccuacaucaacgccgacagcuccaucgaggggaacuacacgcugcgcguggacugcaccccgcugauguacagccucgugcacaaccugaccaaggagcugaagucccccgacgagggcuucgaggggaagagccucuacgaguccuggaccaagaagagcccgucccccgaguucagcggcaugccccggaucuccaagcuggggagcggcaacgacuucgaggucuucuuccagcggcugggcaucgcguccgggcgcgcccgguacacgaagaacugggagaccaacaaguucagcggcuacccccucuaccacuccguguacgagaccuacgagcugguggagaaguucuacgacccgauguucaaguaccaccugaccgucgcccaggugcgcgggggcaugguguucgagcuggccaacagcaucguccuccccuucgacugccgggacuacgccguggugcugcgcaaguacgcggacaagaucuacagcaucuccaugaagcacccccaggagaugaagacguacagcgucuccuucgacagccuguucuccgccgugaagaacuucaccgagaucgccagcaaguucuccgagcggcuccaggacuucgacaagagcaaccccaucgugcugcgcaugaugaacgaccagcugauguuccucgagcgggccuucaucgacccgcuggggcugcccgaccgccccuucuaccggcacgucaucuacgcccccuccagccacaacaaguacgcgggcgaguccuucccggggaucuacgacgcccucuucgacaucgagagcaagguggaccccuccaaggccuggggcgaggugaagcgccagaucuacgucgccgccuucaccgugcaggcggccgccgagacccugagcgagguggccuga

SEQ ID NO: 7 - RNA, 576 nt, Artificial Sequence
  CAP-PSCA(GC)-muag-A64-C30
gggagaaagcuuaccaugaaggccgugcugcucgcgcugcugauggccggccuggcccugcagccggggaccgcccugcugugcuacagcugcaaggcccaggucucgaacgaggacugccugcagguggagaacugcacgcagcugggcgagcagugcuggaccgcccggauccgcgccgugggccugcucaccgugaucagcaagggcugcagccugaacugcguggacgacagccaggacuacuacgugggcaagaagaacaucaccugcugcgacaccgaccugugcaacgccagcggcgcccacgcccugcagcccgcggccgccauccuggcccugcugcccgcccugggccugcugcucuggggccccggccagcugugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuagacaauuggaauu

SEQ ID NO: 8 - RNA, 372 nt, Artificial Sequence
  CDS PSCA(wt)
augaaggcugugcugcuugcccuguugauggcaggcuuggcccugcagccaggcacugcccugcugugcuacuccugcaaagcccaggugagcaacgaggacugccugcagguggagaacugcacccagcugggggagcagugcuggaccgcgcgcauccgcgcaguuggccuccugaccgucaucagcaaaggcugcagcuugaacugcguggaugacucacaggacuacuacgugggcaagaagaacaucacgugcugugacaccgacuugugcaacgccagcggggcccaugcccugcagccggcugcugccauccuugcgcugcucccugcacucggccugcugcucuggggacccggccagcucuag

SEQ ID NO: 9 - RNA, 372 nt, Artificial Sequence
  CDS PSCA(GC)
augaaggccgugcugcucgcccugcugauggcgggccucgcccugcagcccgggaccgcccugcucugcuacagcugcaaggcccaggucuccaacgaggacugccugcagguggagaacugcacccagcugggcgagcagugcuggacggcccggauccgcgcgguggggcuccugaccgucaucagcaagggcugcucccugaacugcguggacgacagccaggacuacuacguggggaagaagaacaucaccugcugcgacaccgaccucugcaacgccuccggcgcccacgcccugcagccggcggccgccauccuggcccuccugcccgcccugggccuccugcugugggggcccggccagcucuga

SEQ ID NO: 10 - RNA, 1224 nt, Artificial Sequence
  CAP-STEAP(GC)-muag-A64-C30
gggagaaagcuuaccauggagagccggaaggacaucaccaaccaggaggagcuguggaagaugaagccgcgccggaaccucgaggaggacgacuaccugcacaaggacacgggcgagaccucgaugcugaagcggcccgugcuccugcaccugcaccagaccgcccacgcggacgaguucgacugcccgagcgagcuccagcacacgcaggagcuguucccgcaguggcaccugcccaucaagaucgccgccaucaucgcgagccucaccuuccuguacacccugcuccgcgaggucauccacccgcuggccacgucgcaccagcaguacuucuacaagaucccgauccuggugaucaacaaggugcuccccauggucagcaucacccugcuggcccucguguaccugccgggggugaucgcggccaucguccagcugcacaacggcaccaaguacaagaaguucccgcacuggcucgacaaguggaugcugacgcggaagcaguucggccugcucagcuucuucuucgccgugcugcacgcgaucuacucgcugagcuaccccaugcggcgcagcuaccgguacaagcuccugaacugggccuaccagcaggugcagcagaacaaggaggacgccuggaucgagcacgacgucuggcggauggagaucuacgugucgcuggggaucgugggccucgcgauccuggcccugcucgccgucaccagcaucccgagcgugucggacagccugaccuggcgcgaguuccacuacauccagagcaagcugggcaucgugucgcuccugcuggggacgauccacgcgcucaucuucgccuggaacaaguggaucgacaucaagcaguucgucugguacaccccgcccaccuucaugaucgccguguuccugccgaucgugguccugaucuucaagagcauccucuuccugccgugccugcggaagaagauccucaagauccggcacggcugggaggacgugacgaagaucaacaagaccgagaucugcagccagcugugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuagacaauuggaauu

SEQ ID NO: 11 - RNA, 1020 nt, Artificial Sequence
  CDS STEAP(wt)
auggaaagcagaaaagacaucacaaaccaagaagaacuuuggaaaaugaagccuaggagaaauuuagaagaagacgauuauuugcauaaggacacgggagagaccagcaugcuaaaaagaccugugcuuuugcauuugcaccaaacagcccaugcugaugaauuugacugcccuucagaacuucagcacacacaggaacucuuuccacaguggcacuugccaauuaaaauagcugcuauuauagcaucucugacuuuucuuuacacucuucugagggaaguaauucacccuuuagcaacuucccaucaacaauauuuuuauaaaauuccaauccuggucaucaacaaagucuugccaaugguuuccaucacucucuuggcauugguuuaccugccaggugugauagcagcaauuguccaacuucauaauggaaccaaguauaagaaguuuccacauugguuggauaaguggauguuaacaagaaagcaguuugggcuucucaguuucuuuuuugcuguacugcaugcaauuuauagucugucuuacccaaugaggcgauccuacagauacaaguugcuaaacugggcauaucaacagguccaacaaaauaaagaagaugccuggauugagcaugauguuuggagaauggagauuuaugugucucugggaauugugggauuggcaauacuggcucuguuggcugugacaucuauuccaucugugagugacucuuugacauggagagaauuucacuauauucagagcaagcuaggaauuguuucccuucuacugggcacaauacacgcauugauuuuugccuggaauaaguggauagauauaaaacaauuuguaugguauacaccuccaacuuuuaugauagcuguuuuccuuccaauuguuguccugauauuuaaaagcauacuauuccugccaugcuugaggaagaagauacugaagauuagacaugguugggaagacgucaccaaaauuaacaaaacugagauauguucccaguuguag

SEQ ID NO: 12 - RNA, 1020 nt, Artificial Sequence
  CDS STEAP(GC)
auggagagccggaaggacaucaccaaccaggaggagcuguggaagaugaagccccgccggaaccucgaggaggacgacuaccugcacaaggacaccggcgagacguccaugcugaagcgcccggugcuccugcaccugcaccagaccgcccacgccgacgaguucgacugccccagcgagcuccagcacacccaggagcuguucccccaguggcaccugcccaucaagaucgcggccaucaucgccucccucaccuuccuguacacgcugcuccgggaggucauccacccgcuggccaccagccaccagcaguacuucuacaagauccccauccuggugaucaacaaggugcuccccauggucuccaucacccugcuggcccucguguaccugcccggggugaucgcggccaucguccagcugcacaacggcaccaaguacaagaaguucccgcacuggcucgacaaguggaugcugacgcgcaagcaguucgggcugcucagcuucuucuucgccgugcugcacgccaucuacucccugagcuaccccaugcggcgcuccuaccgguacaagcuccugaacugggcguaccagcaggugcagcagaacaaggaggacgccuggaucgagcacgacgucuggcgcauggagaucuacgugagccugggcaucguggggcucgccauccuggcccugcucgccgucaccuccauccccagcguguccgacagccugaccuggcgggaguuccacuacauccaguccaagcugggcaucgugagccuccugcugggcaccauccacgcgcucaucuucgccuggaacaaguggaucgacaucaagcaguucgucugguacacgcccccgaccuucaugaucgccguguuccugcccaucgugguccugaucuucaaguccauccucuuccugcccugccugcgcaagaagauccucaagauccggcacgggugggaggacgugaccaagaucaacaagaccgagaucugcagccagcuguga

SEQ ID NO: 13 - RNA, 1353 nt, Artificial Sequence
  CAP-PAP(GC)-muag-A64-C30
gggagaaagcuuaccaugcgggccgccccgcugcuccuggcgcgcgccgccagccugucccucggcuuccuguuccugcucuucuucuggcuggaccggagcgugcuggccaaggagcucaaguucgucacccugguguuccgccacggggaccgguccccgaucgacaccuuccccacggaccccaucaaggagagcuccuggccccagggcuucgggcagcugacccagcucggcauggagcagcacuacgagcugggggaguacauccgcaagcgguaccgcaaguuccugaacgagagcuacaagcacgagcagguguacauccgguccaccgacgucgaccgcacccucaugagcgccaugacgaaccuggcggcccuguucccgcccgagggcguguccaucuggaaccccauccuccuguggcagcccaucccggugcacaccgucccccugagcgaggaccagcuccuguaccugcccuuccggaacugcccccgcuuccaggagcucgaguccgagacccugaagagcgaggaguuccagaagcggcugcacccguacaaggacuucaucgccacccucggcaagcuguccgggcugcacggccaggaccucuucgggaucuggagcaagguguacgacccccuguacugcgaguccgugcacaacuucacgcugcccagcugggccaccgaggacaccaugaccaagcuccgcgagcuguccgagcugagccuccugucccuguacggcauccacaagcagaaggagaagagccggcuccagggcggcguccuggugaacgagauccugaaccacaugaagcgcgcgacgcagauccccagcuacaagaagcucaucauguacuccgcccacgacaccaccgugagcgggcugcagauggcccuggacgucuacaacgggcuccugccgcccuacgccuccugccaccugaccgagcuguacuucgagaagggcgaguacuucguggagauguacuaccggaacgagacgcagcacgagcccuacccccucaugcugccggggugcagccccuccugcccccuggagcgcuucgccgagcucgugggccccgucaucccgcaggacuggagcaccgagugcaugaccaccaacucccaccagggcacggaggacagcaccgacugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuag

SEQ ID NO: 14 - RNA, 1161 nt, Artificial Sequence
  CDS PAP(wt)
augagagcugcaccccuccuccuggccagggcagcaagccuuagccuuggcuucuuguuucugcuuuuuuucuggcuagaccgaaguguacuagccaaggaguugaaguuugugacuuugguguuucggcauggagaccgaagucccauugacaccuuucccacugaccccauaaaggaauccucauggccacaaggauuuggccaacucacccagcugggcauggagcagcauuaugaacuuggagaguauauaagaaagagauauagaaaauucuugaaugaguccuauaaacaugaacagguuuauauucgaagcacagacguugaccggacuuugaugagugcuaugacaaaccuggcagcccuguuucccccagaaggugucagcaucuggaauccuauccuacucuggcagcccaucccggugcacacaguuccucuuucugaagaucaguugcuauaccugccuuucaggaacugcccucguuuucaagaacuugagagugagacuuugaaaucagaggaauuccagaagaggcugcacccuuauaaggauuuuauagcuaccuugggaaaacuuucaggauuacauggccaggaccuuuuuggaauuuggaguaaagucuacgacccuuuauauugugagaguguucacaauuucacuuuacccuccugggccacugaggacaccaugacuaaguugagagaauugucagaauugucccuccugucccucuauggaauucacaagcagaaagagaaaucuaggcuccaaggggguguccuggucaaugaaauccucaaucacaugaagagagcaacucagauaccaagcuacaaaaaacucaucauguauucugcgcaugacacuacugugaguggccuacagauggcgcuagauguuuacaacggacuccuuccucccuaugcuucuugccacuugacggaauuguacuuugagaagggggaguacuuuguggagauguacuaucggaaugagacgcagcacgagccguauccccucaugcuaccuggcugcagccccagcuguccucuggagagguuugcugagcugguuggcccugugaucccucaagacugguccacggaguguaugaccacaaacagccaucaagguacugaagacaguacagauuag

SEQ ID NO: 15 - RNA, 1161 nt, Artificial Sequence
  CDS PAP(GC)
augcgggccgccccgcugcuccuggcgcgcgccgccagccugucccucggcuuccuguuccugcucuucuucuggcuggaccggagcgugcuggccaaggagcucaaguucgucacccugguguuccgccacggggaccgguccccgaucgacaccuuccccacggaccccaucaaggagagcuccuggccccagggcuucgggcagcugacccagcucggcauggagcagcacuacgagcugggggaguacauccgcaagcgguaccgcaaguuccugaacgagagcuacaagcacgagcagguguacauccgguccaccgacgucgaccgcacccucaugagcgccaugacgaaccuggcggcccuguucccgcccgagggcguguccaucuggaaccccauccuccuguggcagcccaucccggugcacaccgucccccugagcgaggaccagcuccuguaccugcccuuccggaacugcccccgcuuccaggagcucgaguccgagacccugaagagcgaggaguuccagaagcggcugcacccguacaaggacuucaucgccacccucggcaagcuguccgggcugcacggccaggaccucuucgggaucuggagcaagguguacgacccccuguacugcgaguccgugcacaacuucacgcugcccagcugggccaccgaggacaccaugaccaagcuccgcgagcuguccgagcugagccuccugucccuguacggcauccacaagcagaaggagaagagccggcuccagggcggcguccuggugaacgagauccugaaccacaugaagcgcgcgacgcagauccccagcuacaagaagcucaucauguacuccgcccacgacaccaccgugagcgggcugcagauggcccuggacgucuacaacgggcuccugccgcccuacgccuccugccaccugaccgagcuguacuucgagaagggcgaguacuucguggagauguacuaccggaacgagacgcagcacgagcccuacccccucaugcugccggggugcagccccuccugcccccuggagcgcuucgccgagcucgugggccccgucaucccgcaggacuggagcaccgagugcaugaccaccaacucccaccagggcacggaggacagcaccgacuga

SEQ ID NO: 16 - RNA, 1860 nt, Artificial Sequence
  CAP-MUC1 5xVNTR(GC)-muag-A64-C30
gggagaaagcuuaccaugacccccggcacccagagcccguucuuccugcuccugcugcucacggugcugaccgucgugaccggguccggccacgccagcuccacccccgggggcgagaaggagacgagcgccacccagcgguccagcgugcccuccagcaccgagaagaacgcggucuccaugaccagcuccgugcugagcucccacagccccggguccggcagcuccacgacccagggccaggacgugacccucgccccggccaccgagcccgccagcggguccgccgcgacguggggccaggacgucaccagcgugcccgugacccgccccgcccuggggagcaccacgccgcccgcccacgacgucaccuccgcccccgacaacaagcccgcgccgggcagcaccgccccccccgcccacggggugaccuccgcccccgacacgcggccggcccccggcagcaccgcgccccccgcccacggcgugaccuccgccccggacacccgccccgcccccgggagcacggccccgccggcgcacggcgucaccuccgcccccgacacccggcccgcccccgggagcaccgccccgcccgcccacggcgugacguccgcgcccgacacccgcccggcccccggcagcaccgccccccccgcccacggggugaccuccgccccggacacgcggcccgcgcccggcagcaccgccccgccggcccacggggucaccuccgcgcccgacaaccgccccgcccuggggagcaccgccccgcccgugcacaacgugaccuccgccagcggcuccgcgagcggguccgccagcacccucguccacaacggcacguccgcccgggccaccaccacccccgccagcaaguccacgcccuucagcaucccgucccaccacagcgacacccccaccacccuggcgucccacagcacgaagaccgacgccuccagcacccaccacuccagcgugcccccgcugaccagcuccaaccacagcacguccccgcagcucagcaccgggguguccuucuucuuccugagcuuccacaucuccaaccugcaguucaacagcucccucgaggaccccagcaccgacuacuaccaggagcugcagcgggacaucuccgagauguuccugcagaucuacaagcagggcggcuuccucgggcugagcaacaucaaguuccgccccggcuccgucguggugcagcugacccucgccuuccgggaggggacgaucaacguccacgacguggagacccaguucaaccaguacaagaccgaggccgccagccgcuacaaccugaccaucuccgacgugagcgucuccgacgugcccuucccguucagcgcgcaguccggcgccggcgugcccggguggggcaucgcccugcucguccuggugugcgugcuggucgcccucgccaucguguaccugaucgcgcuggccgugugccagugccggcgcaagaacuacgggcagcucgacaucuuccccgcccgggacacguaccacccgaugagcgaguacccgaccuaccacacccacggccgcuacgucccccccagcuccaccgaccggagccccuacgagaagguguccgccgggaacggcggcagcucccugagcuacaccaacccggcgguggccgccgccuccgccaaccugugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuag

SEQ ID NO: 17 - RNA, 1668 nt, Artificial Sequence
  CDS MUC1(wt)
augacaccgggcacccagucuccuuucuuccugcugcugcuccucacagugcuuacaguuguuacagguucuggucaugcaagcucuaccccagguggagaaaaggagacuucggcuacccagagaaguucagugcccagcucuacugagaagaaugcugugaguaugaccagcagcguacucuccagccacagccccgguucaggcuccuccaccacucagggacaggaugucacucuggccccggccacggaaccagcuucagguucagcugccaccuggggacaggaugucaccucggucccagucaccaggccagcccugggcuccaccaccccgccagcccacgaugucaccucagccccggacaacaagccagccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaacaggcccgccuugggcuccaccgccccuccaguccacaaugucaccucggccucaggcucugcaucaggcucagcuucuacucuggugcacaacggcaccucugccagggcuaccacaaccccagccagcaagagcacuccauucucaauucccagccaccacucugauacuccuaccacccuugccagccauagcaccaagacugaugccaguagcacucaccauagcucgguaccuccucucaccuccuccaaucacagcacuucuccccaguugucuacuggggucucuuucuuuuuccugucuuuucacauuucaaaccuccaguuuaauuccucucuggaagaucccagcaccgacuacuaccaagagcugcagagagacauuucugaaauguuuuugcagauuuauaaacaaggggguuuucugggccucuccaauauuaaguucaggccaggaucuguggugguacaauugacucuggccuuccgagaagguaccaucaauguccacgacguggagacacaguucaaucaguauaaaacggaagcagccucucgauauaaccugacgaucucagacgucagcgugagugaugugccauuuccuuucucugcccagucuggggcuggggugccaggcuggggcaucgcgcugcuggugcuggucuguguucugguugcgcuggccauugucuaucucauugccuuggcugucugucagugccgccgaaagaacuacgggcagcuggacaucuuuccagcccgggauaccuaccauccuaugagcgaguaccccaccuaccacacccaugggcgcuaugugcccccuagcaguaccgaucguagccccuaugagaagguuucugcagguaacgguggcagcagccucucuuacacaaacccagcaguggcagccgcuucugccaacuuguag

SEQ ID NO: 18 - RNA, 1668 nt, Artificial Sequence
  CDS MUC1(GC)
augacccccggcacccagagcccguucuuccugcuccugcugcucacggugcugaccgucgugaccggguccggccacgccagcuccacccccgggggcgagaaggagacgagcgccacccagcgguccagcgugcccuccagcaccgagaagaacgcggucuccaugaccagcuccgugcugagcucccacagccccggguccggcagcuccacgacccagggccaggacgugacccucgccccggccaccgagcccgccagcggguccgccgcgacguggggccaggacgucaccagcgugcccgugacccgccccgcccuggggagcaccacgccgcccgcccacgacgucaccuccgcccccgacaacaagcccgcgccgggcagcaccgccccccccgcccacggggugaccuccgcccccgacacgcggccggcccccggcagcaccgcgccccccgcccacggcgugaccuccgccccggacacccgccccgcccccgggagcacggccccgccggcgcacggcgucaccuccgcccccgacacccggcccgcccccgggagcaccgccccgcccgcccacggcgugacguccgcgcccgacacccgcccggcccccggcagcaccgccccccccgcccacggggugaccuccgccccggacacgcggcccgcgcccggcagcaccgccccgccggcccacggggucaccuccgcgcccgacaaccgccccgcccuggggagcaccgccccgcccgugcacaacgugaccuccgccagcggcuccgcgagcggguccgccagcacccucguccacaacggcacguccgcccgggccaccaccacccccgccagcaaguccacgcccuucagcaucccgucccaccacagcgacacccccaccacccuggcgucccacagcacgaagaccgacgccuccagcacccaccacuccagcgugcccccgcugaccagcuccaaccacagcacguccccgcagcucagcaccgggguguccuucuucuuccugagcuuccacaucuccaaccugcaguucaacagcucccucgaggaccccagcaccgacuacuaccaggagcugcagcgggacaucuccgagauguuccugcagaucuacaagcagggcggcuuccucgggcugagcaacaucaaguuccgccccggcuccgucguggugcagcugacccucgccuuccgggaggggacgaucaacguccacgacguggagacccaguucaaccaguacaagaccgaggccgccagccgcuacaaccugaccaucuccgacgugagcgucuccgacgugcccuucccguucagcgcgcaguccggcgccggcgugcccggguggggcaucgcccugcucguccuggugugcgugcuggucgcccucgccaucguguaccugaucgcgcuggccgugugccagugccggcgcaagaacuacgggcagcucgacaucuuccccgcccgggacacguaccacccgaugagcgaguacccgaccuaccacacccacggccgcuacgucccccccagcuccaccgaccggagccccuacgagaagguguccgccgggaacggcggcagcucccugagcuacaccaacccggcgguggccgccgccuccgccaaccuguga

SEQ ID NO: 19 - RNA, 1003 nt, Artificial Sequence
  CAP-PSA/KLK3 (GC)-muag-A64-C30-histoneSL
gggagaaagcuuaccaugugggugccggucguguuccugacccucagcgugacguggaucggcgccgcgccccugauccugucgcggaucguggggggcugggagugcgagaagcacagccagcccuggcaggugcugguggccagccgcggccgggccgugugcggcggcgugcuggugcacccccagugggugcugaccgccgcccacugcauccggaacaagagcgucauccugcugggccggcacagccuguuccaccccgaggacaccggccagguguuccaggugagccacagcuucccccacccccuguacgacaugagccuccugaagaaccgguuccugcggcccggcgacgacagcagccacgaccugaugcugcugcggcugagcgagcccgccgagcugaccgacgccgugaaggugauggaccugccgacccaggagcccgcccugggcaccaccugcuacgccagcggcugggggagcaucgagcccgaggaguuccucacccccaagaagcugcagugcguggaccugcacgugaucagcaacgacgugugcgcccaggugcacccccagaaggugaccaaguucaugcugugcgccggccgguggaccggcggcaagagcaccugcagcggcgacagcggcggcccccuggucugcaacggcgugcugcagggcaucaccagcuggggcagcgagcccugcgcccugcccgagcgccccagccuguacaccaagguggugcacuaccggaaguggaucaaggacaccaucguggccaacccgugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 20 - RNA, 2470 nt, Artificial Sequence
  CAP-PSMA/FOLH1 (GC)-muag-A64-C30-histoneSL
gggagaaagcuuaccauguggaaccugcuccacgagaccgacagcgccguggcgacggcccggcgcccgcgguggcugugcgccggcgcccugguccuggccgggggcuucuuccugcugggcuuccuguucggcugguucaucaagucgagcaacgaggccaccaacaucacccccaagcacaacaugaaggccuuccucgacgagcugaaggccgagaacaucaagaaguuccuguacaacuucacccagaucccccaccuggccgggaccgagcagaacuuccagcuggccaagcagauccagagccaguggaaggaguucggccuggacucgguggagcuggcgcacuacgacgugcugcucagcuaccccaacaagacccaccccaacuacaucagcaucaucaacgaggacggcaacgagaucuucaacaccagccuguucgagcccccgccccccggcuacgagaacgugucggacaucgugccccccuucagcgccuucagcccgcagggcaugcccgagggggaccugguguacgugaacuacgcccggacggaggacuucuucaagcuggagcgcgacaugaagaucaacugcagcggcaagaucgugaucgcccgguacggcaagguguuccggggcaacaaggugaagaacgcccagcuggccggggccaagggcgugauccuguacucggaccccgccgacuacuucgcccccggcgugaagagcuaccccgacggcuggaaccugcccggcgggggcguccagcgcggcaacauccucaaccugaacggcgccggcgacccgcugacccccggguaccccgcgaacgaguacgccuaccggcggggcaucgccgaggccgugggccugcccagcauccccgugcacccgaucggcuacuacgacgcccagaagcugcuggagaagaugggcgggagcgccccgcccgacucgagcuggcggggcagccugaaggugcccuacaacgugggccccggcuucaccgggaacuucucgacccagaaggugaagaugcacauccacagcaccaacgaggugacccgcaucuacaacgugaucggcacccugcggggcgccguggagcccgaccgguacgugauccucggcgggcaccgcgacagcuggguguucggcggcaucgacccccagagcggcgccgccgugguccacgagaucgugcggucguucggcacccugaagaaggagggguggcggccccgccggacgauccuguucgccagcugggacgcggaggaguucggccugcugggcagcaccgagugggccgaggagaacagccggcugcugcaggagcggggcguggccuacaucaacgccgacucgagcaucgagggcaacuacacccuccgcguggacugcaccccgcugauguacagccuggugcacaaccugaccaaggagcugaagagccccgacgagggguucgagggcaagucgcuguacgagagcuggaccaagaagagccccucgcccgaguucagcggcaugccccggaucagcaagcugggcagcgggaacgacuucgagguguucuuccagcggcugggcaucgccucgggccgcgcccgguacaccaagaacugggagacgaacaaguucagcggcuacccccucuaccacagcguguacgagaccuacgagcugguggagaaguucuacgaccccauguucaaguaccaccugaccguggcccaggugcggggcgggaugguguucgagcuggccaacagcaucgugcugcccuucgacugccgcgacuacgccgucgugcugcggaaguacgccgacaagaucuacucgaucagcaugaagcacccccaggagaugaagaccuacagcgugagcuucgacucgcuguucagcgcggugaagaacuucaccgagaucgccagcaaguucucggagcggcuccaggacuucgacaagagcaacccgaucgugcugcgcaugaugaacgaccagcugauguuccuggagcgggccuucaucgacccccugggccugcccgaccggcccuucuaccggcacgugaucuacgcccccagcagccacaacaaguacgccggcgagucguucccggggaucuacgacgcccuguucgacaucgagagcaagguggaccccagcaaggccuggggcgaggugaagcgccagaucuacguggccgccuucaccgugcaggccgcggccgagacccugagcgagguggccugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 21 - RNA, 589 nt, Artificial Sequence
  CAP-PSCA (GC)-muag-A64-C30-histoneSL
gggagaaagcuuaccaugaaggccgugcugcucgcgcugcugauggccggccuggcccugcagccggggaccgcccugcugugcuacagcugcaaggcccaggucucgaacgaggacugccugcagguggagaacugcacgcagcugggcgagcagugcuggaccgcccggauccgcgccgugggccugcucaccgugaucagcaagggcugcagccugaacugcguggacgacagccaggacuacuacgugggcaagaagaacaucaccugcugcgacaccgaccugugcaacgccagcggcgcccacgcccugcagcccgcggccgccauccuggcccugcugcccgcccugggccugcugcucuggggccccggccagcugugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 22 - RNA, 1237 nt, Artificial Sequence
  CAP-STEAP1 (GC)-muag-A64-C30-histoneSL
gggagaaagcuuaccauggagagccggaaggacaucaccaaccaggaggagcuguggaagaugaagccgcgccggaaccucgaggaggacgacuaccugcacaaggacacgggcgagaccucgaugcugaagcggcccgugcuccugcaccugcaccagaccgcccacgcggacgaguucgacugcccgagcgagcuccagcacacgcaggagcuguucccgcaguggcaccugcccaucaagaucgccgccaucaucgcgagccucaccuuccuguacacccugcuccgcgaggucauccacccgcuggccacgucgcaccagcaguacuucuacaagaucccgauccuggugaucaacaaggugcuccccauggucagcaucacccugcuggcccucguguaccugccgggggugaucgcggccaucguccagcugcacaacggcaccaaguacaagaaguucccgcacuggcucgacaaguggaugcugacgcggaagcaguucggccugcucagcuucuucuucgccgugcugcacgcgaucuacucgcugagcuaccccaugcggcgcagcuaccgguacaagcuccugaacugggccuaccagcaggugcagcagaacaaggaggacgccuggaucgagcacgacgucuggcggauggagaucuacgugucgcuggggaucgugggccucgcgauccuggcccugcucgccgucaccagcaucccgagcgugucggacagccugaccuggcgcgaguuccacuacauccagagcaagcugggcaucgugucgcuccugcuggggacgauccacgcgcucaucuucgccuggaacaaguggaucgacaucaagcaguucgucugguacaccccgcccaccuucaugaucgccguguuccugccgaucgugguccugaucuucaagagcauccucuuccugccgugccugcggaagaagauccucaagauccggcacggcugggaggacgugacgaagaucaacaagaccgagaucugcagccagcugugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 23 - RNA, 1378 nt, Artificial Sequence
  CAP-PAP (GC)-muag-A64-C30-histoneSL
gggagaaagcuuaccaugcgggccgccccgcugcuccuggcgcgcgccgccagccugucccucggcuuccuguuccugcucuucuucuggcuggaccggagcgugcuggccaaggagcucaaguucgucacccugguguuccgccacggggaccgguccccgaucgacaccuuccccacggaccccaucaaggagagcuccuggccccagggcuucgggcagcugacccagcucggcauggagcagcacuacgagcugggggaguacauccgcaagcgguaccgcaaguuccugaacgagagcuacaagcacgagcagguguacauccgguccaccgacgucgaccgcacccucaugagcgccaugacgaaccuggcggcccuguucccgcccgagggcguguccaucuggaaccccauccuccuguggcagcccaucccggugcacaccgucccccugagcgaggaccagcuccuguaccugcccuuccggaacugcccccgcuuccaggagcucgaguccgagacccugaagagcgaggaguuccagaagcggcugcacccguacaaggacuucaucgccacccucggcaagcuguccgggcugcacggccaggaccucuucgggaucuggagcaagguguacgacccccuguacugcgaguccgugcacaacuucacgcugcccagcugggccaccgaggacaccaugaccaagcuccgcgagcuguccgagcugagccuccugucccuguacggcauccacaagcagaaggagaagagccggcuccagggcggcguccuggugaacgagauccugaaccacaugaagcgcgcgacgcagauccccagcuacaagaagcucaucauguacuccgcccacgacaccaccgugagcgggcugcagauggcccuggacgucuacaacgggcuccugccgcccuacgccuccugccaccugaccgagcuguacuucgagaagggcgaguacuucguggagauguacuaccggaacgagacgcagcacgagcccuacccccucaugcugccggggugcagccccuccugcccccuggagcgcuucgccgagcucgugggccccgucaucccgcaggacuggagcaccgagugcaugaccaccaacucccaccagggcacggaggacagcaccgacugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 24 - RNA, 1885 nt, CAP-MUC1 5xVNTR (GC)-muag-A64-C30-histoneSL
gggagaaagcuuaccaugacccccggcacccagagcccguucuuccugcuccugcugcucacggugcugaccgucgugaccggguccggccacgccagcuccacccccgggggcgagaaggagacgagcgccacccagcgguccagcgugcccuccagcaccgagaagaacgcggucuccaugaccagcuccgugcugagcucccacagccccggguccggcagcuccacgacccagggccaggacgugacccucgccccggccaccgagcccgccagcggguccgccgcgacguggggccaggacgucaccagcgugcccgugacccgccccgcccuggggagcaccacgccgcccgcccacgacgucaccuccgcccccgacaacaagcccgcgccgggcagcaccgccccccccgcccacggggugaccuccgcccccgacacgcggccggcccccggcagcaccgcgccccccgcccacggcgugaccuccgccccggacacccgccccgcccccgggagcacggccccgccggcgcacggcgucaccuccgcccccgacacccggcccgcccccgggagcaccgccccgcccgcccacggcgugacguccgcgcccgacacccgcccggcccccggcagcaccgccccccccgcccacggggugaccuccgccccggacacgcggcccgcgcccggcagcaccgccccgccggcccacggggucaccuccgcgcccgacaaccgccccgcccuggggagcaccgccccgcccgugcacaacgugaccuccgccagcggcuccgcgagcggguccgccagcacccucguccacaacggcacguccgcccgggccaccaccacccccgccagcaaguccacgcccuucagcaucccgucccaccacagcgacacccccaccacccuggcgucccacagcacgaagaccgacgccuccagcacccaccacuccagcgugcccccgcugaccagcuccaaccacagcacguccccgcagcucagcaccgggguguccuucuucuuccugagcuuccacaucuccaaccugcaguucaacagcucccucgaggaccccagcaccgacuacuaccaggagcugcagcgggacaucuccgagauguuccugcagaucuacaagcagggcggcuuccucgggcugagcaacaucaaguuccgccccggcuccgucguggugcagcugacccucgccuuccgggaggggacgaucaacguccacgacguggagacccaguucaaccaguacaagaccgaggccgccagccgcuacaaccugaccaucuccgacgugagcgucuccgacgugcccuucccguucagcgcgcaguccggcgccggcgugcccggguggggcaucgcccugcucguccuggugugcgugcuggucgcccucgccaucguguaccugaucgcgcuggccgugugccagugccggcgcaagaacuacgggcagcucgacaucuuccccgcccgggacacguaccacccgaugagcgaguacccgaccuaccacacccacggccgcuacgucccccccagcuccaccgaccggagccccuacgagaagguguccgccgggaacggcggcagcucccugagcuacaccaacccggcgguggccgccgccuccgccaaccugugaccacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 25 - RNA, 16 nt, Artificial Sequence
  Sequence according to formula (Ic)
ngnnnnnnunnnnncn

SEQ ID NO: 26 - RNA, 26 nt, Artificial Sequence
  Sequence according to formula (IIc)
nnnnnngnnnnnnunnnnncnnnnnn

SEQ ID NO: 27 - RNA, 16 nt, Artificial Sequence
  Sequence according to formula (Id)
ncnnnnnnunnnnngn

SEQ ID NO: 28 - RNA, 26 nt, Artificial Sequence
  Sequence according to formula (IId)
nnnnnncnnnnnnunnnnngnnnnnn

SEQ ID NO: 29 - RNA, 16 nt, Artificial Sequence
  Sequence according to formula (Ie)
dgnnnnnnunnnnnch

SEQ ID NO: 30 - RNA, 26 nt, Artificial Sequence
  Sequence according to formula (IIe)
nnnnndgnnnnnnunnnnnchnnnnn

SEQ ID NO: 31 - RNA, 16 nt, Artificial Sequence
  Sequence according to formula (If)
ngnbyynnunvndncn

SEQ ID NO: 32 - RNA, 26 nt, Artificial Sequence
  Sequence according to formula (IIf)
nnnnnngnbyynnunvndncnnnnnn

SEQ ID NO: 33 - RNA, 16 nt, Artificial Sequence
  Sequence according to formula (Ig)
nghyyydnuhabrdcn

SEQ ID NO: 34 - RNA, 26 nt, Artificial Sequence
  Sequence according to formula (IIg)
nnhnnnghyyydnuhabrdcnnnnnh

SEQ ID NO: 35 - RNA, 16 nt, Artificial Sequence
  Sequence according to formula (Ih)
dghycudyuhasrrcc

SEQ ID NO: 36 - RNA, 26 nt, Artificial Sequence
  Sequence according to formula (IIh)
nhaahdghycudyuhasrrccvhbnh

SEQ ID NO: 37 - DNA, 16 bp, Artificial Sequence
  Sequence according to formula (Ic)
vgyyyyhhthrvvrcb

SEQ ID NO: 38 - DNA, 16 bp, Artificial Sequence
  Sequence according to formula (Ic)
sgyyyttytmarrrcs

SEQ ID NO: 39 - DNA, 16 bp, Artificial Sequence
  Sequence according to formula (Ic)
sgyycttttmagrrcs

SEQ ID NO: 40 - DNA, 16 bp, Artificial Sequence
  Sequence according to formula (Ie)
dgnnnbnnthvnnnch

SEQ ID NO: 41 - DNA, 16 bp, Artificial Sequence
  Sequence according to formula (Ie)
rgnnnyhbthrdnncy

SEQ ID NO: 42 - DNA, 16 bp, Artificial Sequence
  Sequence according to formula (Ie)
rgndbyhythrdhncy

SEQ ID NO: 43 - DNA, 16 bp, Artificial Sequence
  Sequence according to formula (If)
vgyyytyhthrvrrcb

SEQ ID NO: 44 - DNA, 16 bp, Artificial Sequence
  Sequence according to formula (If)
sgyycttytmagrrcs

SEQ ID NO: 45 - DNA, 16 bp, Artificial Sequence
  Sequence according to formula (If)
sgyycttttmagrrcs

SEQ ID NO: 46 - DNA, 16 bp, Artificial Sequence
  Sequence according to formula (Ig)
ggyycttythagrrcc

SEQ ID NO: 47 - DNA, 16 bp, Artificial Sequence
  Sequence according to formula (Ig)
ggcycttytmagrgcc

SEQ ID NO: 48 - DNA, 16 bp, Artificial Sequence
  Sequence according to formula (Ig)
ggctcttttmagrgcc

SEQ ID NO: 49 - DNA, 16 bp, Artificial Sequence
  Sequence according to formula (Ih)
dghyctdythasrrcc

SEQ ID NO: 50 - DNA, 16 bp, Artificial Sequence
  Sequence according to formula (Ih)
ggcyctttthagrgcc

SEQ ID NO: 51 - DNA, 16 bp, Artificial Sequence
  Sequence according to formula (Ih)
ggcycttttmagrgcc

SEQ ID NO: 52 - DNA, 26 bp, Artificial Sequence
  Sequence according to formula (IIc)
hhhhvvgyyyyhhthrvvrcbvhhnn

SEQ ID NO: 53 - DNA, 26 bp, Artificial Sequence
  Sequence according to formula (IIc)
mhmhmsgyyyttytmarrrcsmchhh

SEQ ID NO: 54 - DNA, 26 bp, Artificial Sequence
  Sequence according to formula (IIc)
mmmmmsgyycttttmagrrcsachmh

SEQ ID NO: 55 - DNA, 26 bp, Artificial Sequence
  Sequence according to formula (IIe)
nnnnndgnnnbnnthvnnnchnhnnn

SEQ ID NO: 56 - DNA, 26 bp, Artificial Sequence
  Sequence according to formula (IIe)
nnhhnrgnnnyhbthrdnncydhhnn

SEQ ID NO: 57 - DNA, 26 bp, Artificial Sequence
  Sequence according to formula (IIe)
nhhhvrgndbyhythrdhncyrhhhh

SEQ ID NO: 58 - DNA, 26 bp, Artificial Sequence
  Sequence according to formula (IIf)
hhmhmvgyyytyhthrvrrcbvmhhn

SEQ ID NO: 59 - DNA, 26 bp, Artificial Sequence
  Sequence according to formula (IIf)
mmmmmsgyycttytmagrrcsmchhh

SEQ ID NO: 60 - DNA, 26 bp, Artificial Sequence
  Sequence according to formula (IIf)
mmmmmsgyycttttmagrrcsachmh

SEQ ID NO: 61 - DNA, 26 bp, Artificial Sequence
  Sequence according to formula (IIg)
hhmamggyycttythagrrccvhnnm

SEQ ID NO: 62 - DNA, 26 bp, Artificial Sequence
  Sequence according to formula (IIg)
hhaamggcycttytmagrgccvchhm

SEQ ID NO: 63 - DNA, 26 bp, Artificial Sequence
  Sequence according to formula (IIg)
mmaamggctcttttmagrgccmcymm

SEQ ID NO: 64 - DNA, 26 bp, Artificial Sequence
  Sequence according to formula (IIh)
nhaahdghyctdythasrrccvhbnh

SEQ ID NO: 65 - DNA, 26 bp, Artificial Sequence
  Sequence according to formula (IIh)
hhaamggcyctttthagrgccvmynm

SEQ ID NO: 66 - DNA, 26 bp, Artificial Sequence
  Sequence according to formula (IIh)
hmaaaggcycttttmagrgccrmyhm

SEQ ID NO: 67 - RNA, 13 nt, Artificial Sequence
  Kozak sequence
gccgccaccaugg

SEQ ID NO: 68 - RNA, 15 nt, Artificial Sequence
  general formula of a 3-UTR stabilizing sequence
nccancccnnucncc

SEQ ID NO: 69 - RNA, 44 nt, Artificial Sequence
  3′-UTR of an alpha-globin gene (muag)
gcccgaugggccucccaacgggcccuccuccccuccuugcaccg

SEQ ID NO: 70 - DNA, 24 bp, Artificial Sequence
  Specific histone stem-loop sequence
caaaggctcttttcagagccacca

SEQ ID NO: 71 - RNA, 24 nt, Artificial Sequence
  Specific histone stem-loop sequence
caaaggcucuuuucagagccacca

SEQ ID NO: 72 - protein, 9 aa, Artificial Sequence
  MUC1-derived peptide
SAPDNRPAL

SEQ ID NO: 73 - protein, 8 aa, Artificial Sequence
  Connexin-derived peptide
FEQNTAQP

SEQ ID NO: 74 - protein, 9 aa, Artificial Sequence
  PAP-derived peptide
VSIWNPILL

SEQ ID NO: 75 - protein, 9 aa, Artificial Sequence
  PSMA-derived peptide
SAVKNFTEI

SEQ ID NO: 76 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 77 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 78 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 79 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 80 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 81 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 82 - RNA, 786 nt, Homo sapiens
augugggugccggucguguuccugacccucagcgugacguggaucggcgccgcgccccugauccugucgcggaucguggggggcugggagugcgagaagcacagccagcccuggcaggugcugguggccagccgcggccgggccgugugcggcggcgugcuggugcacccccagugggugcugaccgccgcccacugcauccggaacaagagcgucauccugcugggccggcacagccuguuccaccccgaggacaccggccagguguuccaggugagccacagcuucccccacccccuguacgacaugagccuccugaagaaccgguuccugcggcccggcgacgacagcagccacgaccugaugcugcugcggcugagcgagcccgccgagcugaccgacgccgugaaggugauggaccugccgacccaggagcccgcccugggcaccaccugcuacgccagcggcugggggagcaucgagcccgaggaguuccucacccccaagaagcugcagugcguggaccugcacgugaucagcaacgacgugugcgcccaggugcacccccagaaggugaccaaguucaugcugugcgccggccgguggaccggcggcaagagcaccugcagcggcgacagcggcggcccccuggucugcaacggcgugcugcagggcaucaccagcuggggcagcgagcccugcgcccugcccgagcgccccagccuguacaccaagguggugcacuaccggaaguggaucaaggacaccaucguggccaacccguga

SEQ ID NO: 83 - RNA, 2253 nt, Homo sapiens
auguggaaccugcuccacgagaccgacagcgccguggcgacggcccggcgcccgcgguggcugugcgccggcgcccugguccuggccgggggcuucuuccugcugggcuuccuguucggcugguucaucaagucgagcaacgaggccaccaacaucacccccaagcacaacaugaaggccuuccucgacgagcugaaggccgagaacaucaagaaguuccuguacaacuucacccagaucccccaccuggccgggaccgagcagaacuuccagcuggccaagcagauccagagccaguggaaggaguucggccuggacucgguggagcuggcgcacuacgacgugcugcucagcuaccccaacaagacccaccccaacuacaucagcaucaucaacgaggacggcaacgagaucuucaacaccagccuguucgagcccccgccccccggcuacgagaacgugucggacaucgugccccccuucagcgccuucagcccgcagggcaugcccgagggggaccugguguacgugaacuacgcccggacggaggacuucuucaagcuggagcgcgacaugaagaucaacugcagcggcaagaucgugaucgcccgguacggcaagguguuccggggcaacaaggugaagaacgcccagcuggccggggccaagggcgugauccuguacucggaccccgccgacuacuucgcccccggcgugaagagcuaccccgacggcuggaaccugcccggcgggggcguccagcgcggcaacauccucaaccugaacggcgccggcgacccgcugacccccggguaccccgcgaacgaguacgccuaccggcggggcaucgccgaggccgugggccugcccagcauccccgugcacccgaucggcuacuacgacgcccagaagcugcuggagaagaugggcgggagcgccccgcccgacucgagcuggcggggcagccugaaggugcccuacaacgugggccccggcuucaccgggaacuucucgacccagaaggugaagaugcacauccacagcaccaacgaggugacccgcaucuacaacgugaucggcacccugcggggcgccguggagcccgaccgguacgugauccucggcgggcaccgcgacagcuggguguucggcggcaucgacccccagagcggcgccgccgugguccacgagaucgugcggucguucggcacccugaagaaggagggguggcggccccgccggacgauccuguucgccagcugggacgcggaggaguucggccugcugggcagcaccgagugggccgaggagaacagccggcugcugcaggagcggggcguggccuacaucaacgccgacucgagcaucgagggcaacuacacccuccgcguggacugcaccccgcugauguacagccuggugcacaaccugaccaaggagcugaagagccccgacgagggguucgagggcaagucgcuguacgagagcuggaccaagaagagccccucgcccgaguucagcggcaugccccggaucagcaagcugggcagcgggaacgacuucgagguguucuuccagcggcugggcaucgccucgggccgcgcccgguacaccaagaacugggagacgaacaaguucagcggcuacccccucuaccacagcguguacgagaccuacgagcugguggagaaguucuacgaccccauguucaaguaccaccugaccguggcccaggugcggggcgggaugguguucgagcuggccaacagcaucgugcugcccuucgacugccgcgacuacgccgucgugcugcggaaguacgccgacaagaucuacucgaucagcaugaagcacccccaggagaugaagaccuacagcgugagcuucgacucgcuguucagcgcggugaagaacuucaccgagaucgccagcaaguucucggagcggcuccaggacuucgacaagagcaacccgaucgugcugcgcaugaugaacgaccagcugauguuccuggagcgggccuucaucgacccccugggccugcccgaccggcccuucuaccggcacgugaucuacgcccccagcagccacaacaaguacgccggcgagucguucccggggaucuacgacgcccuguucgacaucgagagcaagguggaccccagcaaggccuggggcgaggugaagcgccagaucuacguggccgccuucaccgugcaggccgcggccgagacccugagcgagguggccuga

SEQ ID NO: 84 - RNA, 372 nt, Homo sapiens
augaaggccgugcugcucgcgcugcugauggccggccuggcccugcagccggggaccgcccugcugugcuacagcugcaaggcccaggucucgaacgaggacugccugcagguggagaacugcacgcagcugggcgagcagugcuggaccgcccggauccgcgccgugggccugcucaccgugaucagcaagggcugcagccugaacugcguggacgacagccaggacuacuacgugggcaagaagaacaucaccugcugcgacaccgaccugugcaacgccagcggcgcccacgcccugcagcccgcggccgccauccuggcccugcugcccgcccugggccugcugcucuggggccccggccagcuguga

SEQ ID NO: 85 - RNA, 1020 nt, Homo sapiens
auggagagccggaaggacaucaccaaccaggaggagcuguggaagaugaagccgcgccggaaccucgaggaggacgacuaccugcacaaggacacgggcgagaccucgaugcugaagcggcccgugcuccugcaccugcaccagaccgcccacgcggacgaguucgacugcccgagcgagcuccagcacacgcaggagcuguucccgcaguggcaccugcccaucaagaucgccgccaucaucgcgagccucaccuuccuguacacccugcuccgcgaggucauccacccgcuggccacgucgcaccagcaguacuucuacaagaucccgauccuggugaucaacaaggugcuccccauggucagcaucacccugcuggcccucguguaccugccgggggugaucgcggccaucguccagcugcacaacggcaccaaguacaagaaguucccgcacuggcucgacaaguggaugcugacgcggaagcaguucggccugcucagcuucuucuucgccgugcugcacgcgaucuacucgcugagcuaccccaugcggcgcagcuaccgguacaagcuccugaacugggccuaccagcaggugcagcagaacaaggaggacgccuggaucgagcacgacgucuggcggauggagaucuacgugucgcuggggaucgugggccucgcgauccuggcccugcucgccgucaccagcaucccgagcgugucggacagccugaccuggcgcgaguuccacuacauccagagcaagcugggcaucgugucgcuccugcuggggacgauccacgcgcucaucuucgccuggaacaaguggaucgacaucaagcaguucgucugguacaccccgcccaccuucaugaucgccguguuccugccgaucgugguccugaucuucaagagcauccucuuccugccgugccugcggaagaagauccucaagauccggcacggcugggaggacgugacgaagaucaacaagaccgagaucugcagccagcuguga

SEQ ID NO: 86 - protein, 555 aa, Artificial Sequence
  MUC1 Protein 5xVNTR
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 87 - DNA, 3768 bp, Homo sapiens
atgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtag